(19) United States
(12) Patent Application Publication
(10) Pub. No.: US 2011/0319415 A1
(43) Pub. Date: Dec. 29, 2011

(54) Susceptibility to HSP90-Inhibitors

(75) Inventors: Roman Thomas, Bornheim (DE); Kathrin Maring, Bonn (DE); Thomas Zander, Bonn (DE); Peter Frommolt, Koln (DE); Kwok-kin Wong, Arlington, MA (US)

(73) Assignees: Universit+e,uml a+ee t zu K+e,uml o+ee ln, Kö'ln (DE); Max-Planck-Gesellschaft zur F+e,uml o+ee rderung der Wissens, München (DE)

(21) Appl. No.: 13/059,557

(22) PCT Filed: Aug. 17, 2009
(86) PCT No.: PCT/EP2009/060640

§ 371 Date: Jun. 8, 2011

Related U.S. Application Data
(60) Provisional application No. 61/102,190, filed on Oct. 2, 2008.

(30) Foreign Application Priority Data
Aug. 18, 2008 (EP) 08014656.6

(51) Int. Cl. A61K 31/395; C12Q 1/68; G01N 33/567; A61K 31/5377; A61P 35/00; A61P 35/02; C40B 30/04; C12Q 1/02

(52) U.S. Cl. 514/236.8; 506/9; 435/6.11; 435/29; 514/183; 435/7.23

(57) ABSTRACT

The present invention relates to a method of selecting (a) cell(s), (a) tissue(s) or (a) cell culture(s) with susceptibility to an HSP90 inhibitor. Also a method for determining the responsiveness of a mammalian tumor cell or cancer cell to treatment with an HSP90 inhibitor is described herein. In particular, the present invention provides for an in vitro method for the identification of a responder for or a patient sensitive to an HSP90 inhibitor and uses of an oligo- or polynucleotide capable of detecting (an) activating mutation(s) in the KRAS gene are provided. The present invention also relates to a method of monitoring the efficacy of a treatment of a cancer characterized by the presence of at least one activating mutation in the KRAS gene, and. optionally, in the EGFR gene and/or the BRAF gene. In addition, a method of predicting the efficacy of a cancer treatment is described, in particular in a cancer that is characterized by the presence of at least one activating mutation in the KRAS gene. and. optionally, in the EGFR gene and/or the BRAF gene. Also the use of a (transgenic) non-human animal or a (transgenic) cell having at least one activating mutation in the KRAS gene, and, optionally, in the EGFR gene and/or the BRAF gene for screening and/or validation of a medicament for the treatment of said cancer is described and a kit useful for carrying out the methods described herein is provided.

The present invention relates to a method of selecting (a) cell(s), (a) tissue(s) or (a) cell culture(s) with susceptibility to an HSP90 inhibitor. Also a method for determining the responsiveness of a mammalian tumor cell or cancer cell to treatment with an HSP90 inhibitor is described herein. In particular, the present invention provides for an in vitro method for the identification of a responder for or a patient sensitive to an HSP90 inhibitor and uses of an oligo- or polynucleotide capable of detecting (an) activating mutation(s) in the KRAS gene are provided. The present invention also relates to a method of monitoring the efficacy of a treatment of a cancer characterized by the presence of at least one activating mutation in the KRAS gene, and, optionally, in the EGFR gene and/or the BRAF gene. In addition, a method of predicting the efficacy of a cancer treatment is described, in particular in a cancer that is characterized by the presence of at least one activating mutation in the KRAS gene, and, optionally, in the EGFR gene and/or the BRAF gene. Also the use of a (transgenic) non-human animal or a (transgenic) cell having at least one activating mutation in the KRAS gene, and, optionally, in the EGFR gene and/or the BRAF gene for screening and/or validation of a medicament for the treatment of said cancer is described and a kit useful for carrying out the methods described herein is provided.

The dynamics of ongoing efforts to fully annotate the genomes of all major cancer types is reminiscent of that of the Human Genome Project. The analysis of somatic gene copy number alterations and gene mutations associated with cancer (both are here referred to as lesions) will thus provide the genetic landscape of human cancer in the near future. The medical implications of these endeavors are exemplified by the success of molecularly targeted cancer therapeutics in genetically defined tumors: the ERBB2/Her2-targeted antibody trastuzumab shrinks tumors in women with ERBB2-amplified breast cancer (Slamon et al., 2001); the ABL/KIT/PDGFR inhibitor imatinib induces responses in patients with chronic myeloid leukemia carrying the BCR/ABL translocation. (Druker et al., 2001a; Druker et al., 2001b) as well as in gastrointestinal stromal tumors and melanomas hearing (see Hodi et al NEJM 2008) mutations in KIT or PDGFRA (Heinrich et al., 2003); finally, EGFR-mutant lung tumors are highly sensitive to the EGFR inhibitors gefitinib and erlotinib (Lynch et al., 2004; Paez et al., 2004; Pao et al., 2004). In most cases, such discoveries were made after the completion of clinical trials; and as yet no robust mechanism currently exists that permits systematic identification of lesions causing therapeutically relevant oncogene dependency prior to initiation of clinical trials involving patients. Thus, somatic genetic alterations (lesions) in cancer have been causally linked with response to targeted therapeutics as they frequently expose a specific dependence on activated oncogenic signalling pathways. However, no tools currently exist to systematically link such lesions to therapeutic vulnerability.

K-Ras protein is a GTP-ase involved in regulating cell division. Patients suffering from cancer with mutations in the KRAS gene (e.g. non-small lung cancer, pancreatic cancer, colorectal cancer, breast cancer, leukemias, prostate cancer, lymphomas, brain tumors, pediatric tumors, sarcomas) have a bad prognosis and, in particular, a very low survival rate. Since these disorders cannot be effectively treated the identification of drugs/compounds to which tumors/tumor cell(s) with said KRAS mutations are susceptible is highly desirable. In particular, the identification prior to initiation/completion of clinical trials is desirable avoiding trials involving humans. The term “KRAS” as used herein relates to K-Ras, k-ras and the like and the terms are used as synonyms. However, the term “KRAS” relates mostly to the gene encoding the K-Ras GTP-ase, whereas the term “K-ras”, “K-Ras” or “K-RAS” mostly denotes the encoded protein.

Cancer cell lines may be used in corresponding in vitro experiments for identification of drugs/compounds to which tumors/tumor cell(s) with the above-mentioned KRAS mutations are susceptible; yet, the validity and clinical interpretability of these widely used models have been questioned. In addition, cell lines are frequently thought to be genomically disarrayed and unstable and therefore likely poorly representative of primary tumors. Furthermore, the genetic diversity of histopathologically defined classes of tumors is often substantial; e.g., the clinical tumor entity non-small cell lung cancer (NSCLC) comprises EGFR- and KRAS-mutant lung adenocarcinomas as well as KRAS-mutant squamous-cell lung cancers. Thus, any representative pre-clinical model would need to capture the nature of lesions of primary tumors as well as their distribution in the histopathologically defined cohort.

Recent reports have credentialed the use of cancer cell lines in preclinical drug target validation experiments (Lin et al., 2008; McDermott et al., 2007; Neve et al., 2006; Solit et al., 2006). However, these studies either employed cell line collections of mixed cancer lineage (Solit et al., 2006), were focused on large-scale genomic analysis of cancer cell line collections (Lin et al., 2008) or established high-throughput cell line profiling to define cell lines with exquisite inhibitor sensitivity without genomic analyses (McDermott et al., 2007). Thus, somatic genetic alterations (lesions) in cancer have been causally linked with response to targeted therapeutics as they frequently expose a specific dependence on activated oncogenic signalling pathways. However, no tools currently exist to systematically link such lesions to therapeutic vulnerability. Accordingly, an identification of compounds/drugs to which tumors are susceptible is often time-consuming and cost-intensive since these compounds/drugs may only be identified after completion of clinical trials. Also a specific tumor entity (e.g “lung cancer”) may comprise various tumor types characterized by different genomic and/or transcriptional profiles. Therefore, even “same” tumors (tumors belonging to the same tumor entity, such as lung cancer) may not be amenable to treatment with the same drug/compound. Therefore, many patients with tumors belonging to the same tumor entity would profit from an identification of markers/predictors for susceptibility of the specific tumor type to (a) certain drug(s)/compound(s).

Thus, the technical problem underlying the present invention is the provision of means and methods for the evaluation of cells, in particular tumor cells, for their susceptibility or responsiveness to anti-cancer treatment.

The technical problem is solved by provision of the embodiments characterized in the claims.

Accordingly, the present invention relates to a method of selecting (a) cell(s), (a) tissue(s) or (a) cell culture(s) with susceptibility to an HSP90 inhibitor, comprising the steps: (a) determining the presence of at least one mutation in the KRAS gene in said cell, tissue or cell culture; and (b) selecting (a) cell(s), tissue(s) or cell culture(s) with at least one mutation in the KRAS gene. The method may additionally comprise (i) contacting said cell(s), tissue(s) or cell culture(s) with an HSP90 inhibitor; and (ii)) evaluating viability of said cell(s), tissue(s) or cell culture(s) contacted with an HSP90 inhibitor. It is of note that steps (i) and (ii) may be performed prior to step (a) but also after step (a) or, optionally after step (b). Said steps (i) and (ii) may in particular serve as further experimental proof that the selected cell, tissue or cell culture that comprises (an) activating KRAS mutation(s) is susceptible in its viability to an HSP90 inhibitor. The KRAS mutation(s) is/are therefore and preferably “activating mutations”

As used herein, the term “cell, tissue and cell culture” is not only limited to isolated cells, tissues and cell cultures but also comprises the use of samples, i.e. biological, medical or pathological samples that consist of fluids that comprise such cells, tissues or cell cultures. Such a fluid may be a body fluid or also excrements and may also be a culture sample, like the culture medium from cultured cells or cultured tissues. The body fluids may comprise, but are not limited to blood, serum, plasma, urine, saliva, synovial fluid, spinal fluid, cerebrospinal fluid, tears, stool and the like.

Accordingly, the gist of the present invention lies in the fact that a method is provided that allows for the determination of the susceptibility of a given cell, tissue or cells in a tissue, (or a cell culture or individual cells in such a cell culture, or as will be explained below, (a) cell(s) in a biological/medical/pathological sample) for the anti-cancer or anti-proliferative treatment with an HSP90 inhibitor. As detailed in the appended examples, it was surprisingly found that cells that comprise an activating mutation in the KRAS gene are in particular susceptible to the treatment with an HSP90 inhibitor. The examples provided herein also show in vivo that HSP90 inhibitors can successfully be employed in the treatment of KRAS-related/KRAS-driven cancers, like KRAS-driven lung adenocarcinomas, i.e. cancers that have an activating mutation in the KRAS gene.

Therefore, the present invention does not only provide for a method for selecting cells/tissues/cell cultures which are susceptible to an HSP90 inhibitor, but also for an in vitro method for assessing an individual, i.e. a human or animal patient, for its potential responsiveness to an anti-cancer or anti-proliferate treatment with an HSP90 inhibitor. The present invention provides not only for the possibility to select cells, tissues and cell cultures that are susceptible for HSP90 inhibitor treatment (i.e. the selection of e.g. research tools whereon novel HSP90 inhibitors may be tested or which are useful in screening methods for compounds that are suspected to function as a HSP90 inhibitor) but also for a method to evaluate whether a given patient, preferably a human patient, in need of treatment but also prevention of a proliferative disease, is a responder for HSP90 inhibitor treatment. Most preferably, the responsiveness of a given patient to the following HSP90 inhibitors is tested: geldanamycin or a derivative thereof, in particular 17-AAG or IPI-504. These and further HSP90 inhibitors that may be tested are described herein below in more detail.

The selection method of a HSP90 inhibitor responding cell or a responding patient comprises a step wherein (a) cell(s), tissue(s) or cell culture(s) with at least one mutation in the KRAS gene is selected. Said cell/tissue may also be derived from a human sample, or from a body fluid that comprises such a cell, for example a cancer cell. Said activating mutation is indicative for susceptibility to an HSP90 inhibitor. The term “activating mutation” used herein refers to a mutation in a gene, in particular in the KRAS gene, which leads to an increased activity of the corresponding gene product, i.e. the protein, in particular the KRAS protein. Methods for measuring the (increased) activity of a protein, in particular the KRAS protein, are known in the art and also described herein below. Also exemplary (activating) mutations of the KRAS gene are described herein below. Again, (activating) mutations are preferred in the context of the present invention.

As pointed out above, and in an alternative embodiment, the present invention relates in particular to a method for determining the responsiveness of a mammalian tumor cell or cancer cell to treatment with an HSP90 inhibitor, said method comprising determining the presence of at least one mutation in the KRAS gene in said tumor cell, wherein said mutation is indicative of whether the cell is likely to respond or is responsive to the treatment. Such a determination may take place on an individual, isolated tumor cell. Such an evaluation may also be carried out on biological/medical/pathological samples, like body fluids, isolated body tissue samples and the like, wherein said samples preferably comprise cells or cell debris to be analyzed.

As pointed out in the technical problem above, there is a need in the art for markers, which can predict the outcome of an anti-cancer therapy with HSP90 inhibitors prior to and during treatment. There is a need for stratification of patients who are to be subjected to or are being subjected to an anti-cancer therapy with HSP90 inhibitors and distinguishing between HSP90 inhibitor “responder” and “non-responder” patients.

Subject of the present invention is a method for diagnosing an individual who is to be subjected to or is being subjected to an anti-cancer treatment or an anti-proliferative treatment to asses the responsiveness to HSP90 inhibitor prior, during and/or after HSP90 inhibitor treatment which comprises the steps of (a) detection of at least one (activating) mutation in the KRAS gene in a biological/medical/pathological sample wherein the presence of said at least one (activating) mutation in said KRAS gene is indicative for the responsiveness to HSP90 inhibitor treatment prior, during and after treatment with such an HSP90 inhibitor; and (b) sorting the individual into responder or Non-responder based on detection of said at least one (activating) mutation in the KRAS gene. Preferably, said at least one mutation in the KRAS gene is an activating mutation as defined herein.

Thus, the invention provides for the first time markers which can predict the outcome of an anti-cancer/anti-proliferative treatment with an HSP90 inhibitor prior to treatment in addition to during and/or after treatment.

The present invention solves the above identified technical problem since, as documented herein below and in the appended examples, it was surprisingly found that the presence of at least one mutation in the KRAS gene in (a) cell(s), (a) tissue(s) or (a) cell culture (or in a biological sample comprising cells or cell debris) is highly predictive for susceptibility of said cell(s), tissue(s) or cell culture(s) (or the individual who provided said biological sample) to an HSP90 inhibitor.

Presently, tumors which are characterized by the presence of at least one mutation in the KRAS gene can not be effectively treated, since a specific therapy for said tumors does not exist. Accordingly, patients suffering from cancer or a neoplastic disorder characterized by the presence of at least one mutation in the KRAS gene have a particularly a bad prognosis; see Eberhardt (2005a), J. Clin. Oncol. Non-limiting examples of said neoplastic disorders or cancers are, inter alia, non-small cell lung cancer, lung adenocarcinoma, pancreatic cancer, colorectal cancer, breast cancer, head and neck cancer, ovarian cancer, endometrial cancer, gastrointestinal cancer (including gastric and esophageal cancer), renal cell cancer, urinary tract carcinomas, leukemias, prostate cancer, lymphomas, melanomas, brain tumors, pediatric tumors or sarcomas. All these disorders are well known in the art. Generally, a person skilled in the art will be aware of further exemplary neoplastic disorders/diseases, e.g. neuroendocrine tumor, teratoma or any other type of neoplastic disorders/diseases. The present invention is not limited to the assessment of treatment success of malignant neoplastic disorders/diseases with HSP90 inhibitors but is also envisaged in the assessment of non-malignant neoplastic disorders with HSP90 inhibitors. Non-malignant, neoplastic disorders are, for examples, lipomas, fibromas, myomas, polyps, warts, condylomata and the like. Yet, a particular focus of the present invention is the assessment of potential treatment success with HSP90 inhibitors in malignant neoplastic diseases/cancers/malignant tumors.

In the present invention, mutations in the KRAS gene were surprisingly identified as markers/predictors for responsiveness to treatment with an HSP90 inhibitor or for susceptibility to an HSP90 inhibitor. The terms “marker” and “predictor” can be used interchangeably and refer to specific allele variants of genes, in particular the KRAS gene and, optionally the EGFR and/or the BRAF gene, which are characterized by the presence of mutations. Exemplary mutations in these genes are described herein below. The presence of these marker/predictor as defined herein correlates significantly (p<0.05) with a responsiveness to treatment with an HSP90 inhibitor/susceptibility to an HSP90 inhibitor. In particular embodiments, any activating KRAS mutation is predictive for a successful treatment (or prevention) of a neoplastic growth (like in cancer) with an HSP90 inhibitor.

The identification of mutations in the KRAS gene as markers for susceptibility of tumor cell(s) to an HSP90 inhibitor, in particular 17-AAG, provides for the first time a therapeutic approach for patients suffering from cancer characterized by the presence of (a) mutation(s) in the KRAS gene. Treatment of patients with an HSP90 inhibitor may lead to an increase in clinical response rate of, for example, at least 80% and an increase in survival by about 100%. Such an increase in the response rate and survival rate has been shown in the prior art in context of EGFR-mutated lung carcinomas treated with EGFR inhibitors erlotinib and gefitinib.

HSP90 is involved in the maturation of mutated oncogenes, such as mutated BRAF and mutated EGFR (Shimamura (2005) Cancer Res; Grbovic (2005) PNAS). In the prior art, studies have been performed in order to substantiate the assumption that tumor cells harboring activating mutations in the RAS-RAF pathway are dependent on chaperonage with Hsp90 (Banerji et al., 2008; Hostein et al., 2001). Tumors with mutations in the BRAF gene and the EGFR gene are highly sensitive to HSP90 inhibitors since growth and development of these tumors depend on HSP90 activity in maturation of these mutated BRAF and EGFR proteins. The prior art has speculated that the presence of mutations in BRAF genes and EGFR genes could be considered as a predictive marker for the susceptibility to an HSP90 inhibitor.

A clinical trial (NCT00431015) investigates the safety and efficacy of the HSP90 Inhibitor IPI-504 treatment of patients suffering from non-small cell lung cancer Smith (Drug Discovery Today: Therapeutic Strategies, 2008). However, the KRAS mutations have neither been described nor proposed in the art as markers for susceptibility of (a) tumor cell(s)/(a) tumor(s) to an HSP90 inhibitor. To the contrary, KRAS mutations have only been described as predictive markers for therapy resistance in the treatment of cancer with chemotherapy alone or in combination with EGFR inhibitors (Pao et al., 2005b), such as erlotinib (Eberhard (2005) J Clin Oncol. 23, 5900-5909), i.e. the art has described KRAS mutations are described as negative markers for susceptibility to EGFR inhibitors. In other words, the art considers tumor cells/tumors characterized by the presence of KRAS mutations as resistant to treatment with EGFR inhibitors, such as erlotinib or gefitinib.

HSP90 is not even known to be involved in the maturation of mutated KRAS proteins. It was merely known in the art that HSP90 may regulate activation of K-RAS dependent signaling via its client protein BRAF (client proteins being proteins stabilized by HSP90). However, HSP90 stabilizes an abundance of such “client proteins”.

Previous studies set out in melanoma patients (Banerji U et al.; Mol Cancer Ther, April; 7(4):737-9 2008) implicated NRAS- and BRAF-mutations as potential biomarkers for the response of HSP90 inhibitors. However, the interpretation of the data is limited due to the fact that only six patients were studied. In detail, two patients with metastatic melanoma were reported to have responded to HSP90 inhibition. One of the tumors carried a mutation in BRAF while the other tumor had an NRAS mutation (Banerji et al., Mol Cancer Ther, April; 7(4):737-9 2008). However, given the widespread distribution of BRAF and NRAS mutations which occur in approximately 45-60% (BRAF) and 20-30% (NRAS) of all melanomas, the fact that two patients responded to this treatment does not support the conclusion that such tumors are enriched in the subset of patients that respond to HSP90 inhibition. By contrast, the findings of the present invention, i.e., that KRAS mutant tumors are especially sensitive to HSP90 inhibition is based on an evaluation of a set of cancers that represent the natural distribution of genetic abnormalities in lung cancer. In this set of tumors, KRAS mutant tumors are enriched in the subset of cancers that were sensitive to HSP90 inhibition.

Furthermore, the finding of the present invention that KRAS is a novel HSP90 client protein is unexpected and surprising. Typical HSP90 client proteins are large proteins with a complex tertiary structure that require extensive folding and processing. The mutant BRAF kinase is such a prototypical example. By contrast, the KRAS protein is a very small protein that does not require extensive structural remodeling in order to become activated. It is to be noted, that KRAS and NRAS, even though they share structural similarities, are independent proteins. In fact, oncogenic transformation by either one of the mutant forms of these proteins requires specific cellular context. For example, BRAF, EGFR and HER2 (i.e. highly complex proteins) are known chaperone targets, since chaperones are essential in correct folding of such large and complex proteins. However, KRAS, a small protein with a relatively simple tertiary structure is not described (and not even speculated on) in the art as a potential HSP90 client/substrate.

It is explained below that mutant KRAS cannot be grouped together with mutant BRAF or EGFR as HSP90 client proteins and would, thus, not be considered by a skilled person as a putative HSP90 client protein.

Furthermore, it is shown herein that significant differences between mutant KRAS and the published results on mutant BRAF or EGFR (Shimamura T et al. Can Res 2005; Grbovic O M et al. PNAS 2006) exist. This demonstrates that mutant KRAS cannot be grouped together with mutant BRAF or EGFR as HSP90 client proteins. To the contrary, it is only found herein that KRAS appears to be a novel, atypical client of Hsp90. It is demonstrated in the appended experimental section that mutant KRAS is bound to Hsp90 and is not depleted from the complex by treatment with an HSP90 inhibitor (17-AAG). As shown in the appended examples mutated as well as unmutated KRAS binds to Hsp90; however binding to HSP90 is not inhibited by an HSP90 inhibitor such as 17-AAG. Using the same conditions it was found herein that mutant EGFR is depleted from the complex by 17-AAG treatment as has been suggested previously (Shimamura T et al.; Can Res 2005). Furthermore. KRAS levels are also not modulated outside of the complex.

However, both c-Raf and AKT levels are modulated by treatment with an HSP90 inhibitor (17-AAG) suggesting, without being bound by theory, that KRAS-mutant cells are killed by HSP90 inhibition because the HSP90 inhibitor depletes two major signaling pathways downstream of KRAS (c-Raf and AKT).

Without being bound by theory, it is believed that KRAS proteins (and, in particular. KRAS proteins with activating mutations as described herein) are a client/substrate of HSP90 which assists in the folding of (activated) KRAS proteins. It is conceivable that HSP90 prevents via its chaperone activity mutated KRAS proteins (i.e. KRAS having activating mutations) from being degraded; thus, an increased level of (an) activated KRAS protein(s) is maintained. This increased level of (an) activated KRAS protein(s) may contribute to the development and/or enhancement of the herein described cancers (e.g. rise in tumors or enhanced tumor growth). In accordance with the above, it is believed that the herein disclosed HSP90 inhibitors prevent correct folding of activated/mutated KRAS proteins via interfering with HSP90 chaperone activity, thus decreasing the level of activated/mutated KRAS proteins. It is apparent that HSP90 inhibitors are, therefore, useful in the treatment of the herein disclosed cancers and in the herein provided means and methods.

Again, it is of note that mutated. KRAS proteins have not been described as HSP90 clients and that activating KRAS mutations accordingly have not been described or proposed to be associated with a susceptibility of tumor/cancer cells to HSP90 inhibitors. For example, Smith ((Drug Discovery Today: Ther Strategies, 2008) described that inhibitors exhibit pleiotropic changes as a consequence of the wide range of HSP90 client proteins and the diverse cellular role of HSP90. While prior studies have focused on the analysis of HSP90-dependent maturation of the proto-oncogene Akt, none of these studies has systematically analyzed the sensitivity of KRAS-mutant cells to HSP90 inhibition. Furthermore, the lack of large cell line collections that are heavily annotated genetically has hampered the ability to perform large-scale preclinical analyses into genotype-phenotype relationship. It is the power of the herein described means and methods involving a large collection of tumor cell lines of one particular tumor type that has been characterized in depth which allows the identification of markers for susceptibility/responsiveness to the treatment with an HSP90 inhibitor.

For example, Smith (Drug Discovery Today: Therapeutic Strategies, 2008) describe a positive correlation between NQO1 expression and 17-AAG sensitivity. Solit (2008) Drug Discovery Today 13, 38-43 describes the relevance of patient selection in the design of HSP90 inhibitor trials. Solit goes on to say that only limited animal studies have been performed with HSP90 inhibitors and that the assumed ability of 17-AAG or other HSP90 inhibitors to degrade putative HSP90 clients has yet to be confirmed by in vivo experiments.

Accordingly, KRAS mutations are never mentioned in context with susceptibility to HSP90 inhibitors and mutated KRAS proteins are not known as a target of HSP90. Accordingly, the identification of (a) KRAS mutation(s) as markers for susceptibility to an HSP90 inhibitor was unexpected and not anticipated by the prior art.

As mentioned, patients suffering from cancer with (a) mutation(s) in the KRAS gene have a particularly low survival rate and a had prognosis and known therapies are not effective. These patients will, therefore, profit enormously from the herein described identification of mutations in the KRAS gene as markers for susceptibility to an HSP90 inhibitor. Further, the herein described methods allow the identification of responders for/patients sensitive to an HSP90 prior to initiation of clinical trials involving patients.

The present invention is illustrated by the experiments described in the appended Example. The experiments show the surprising identification of (a) KRAS mutations) as a predictor of sensitivity to HSP90 inhibitors, in particular to geldanamycin-derivatives such as 17-AAG. Said KRAS mutation(s) were identified as (a) predictor(s)/marker(s) for susceptibility to an Hsp90 inhibitor using the representative NSCLC (non small cell lung cancer) cell line collection as demonstrated in the appended example. This finding was independently validated in the publicly available NCI-60 cell line collection (Garraway et al., 2005; Shoemaker, 2006) (p<0.05). The NCI-60 cell line collection comprises cell lines of diverse tumors and represents most human tumor types. Thus, KRAS mutations are general predictors/markers for susceptibility to an HSP90 inhibitor/responsiveness to treatment with an HSP90 inhibitor. Accordingly, not only NSCLC cancer but any cancer characterized by the presence of (a) mutation(s) in the KRAS gene (e.g. non-small cell lung cancer, lung adenocarcinoma, pancreatic cancer, colorectal cancer, breast cancer, leukemias, prostate cancer, lymphomas, brain tumors, pediatric tumors, sarcomas) can be treated in accordance with the present invention. Preferably, said mutation(s) in the KRAS gene is an activating one. Therefore, in the embodiments described herein it is most preferred that activated KRAS gene mutation are searched, identified and/or evaluated.

Animal data is provided in the appended examples which demonstrate that activating mutations are indeed indicative for susceptibility to an HSP90 inhibitor. A transgenic mouse model is used in the experimental section below; these transgenic lox-stop-loxKRAS_G12D mice develop aggressive lung adenocarcinoma upon expression of a mutant KRAS allele in the lung (Jackson E L et al.; Genes Dev 2001). The lung tumor-bearing lox-stop-loxKRAS_G12D mice were treated with the exemplary HSP90 inhibitor 17-DMAG (described and defined herein), a more soluble 17-AAG derivative. It is of note that one-week treatment led to tumor regressions in 3 of 4 mice. Although responses were transient, these results clearly confirm the findings described herein using cell lines that tumors with activating KRAS mutations respond to HSP90 inhibitors. This shows that the findings based on the large-scale cell-based screening experiments involving genomically annotated cell line panels are confirmed in vivo.

One advantage of the present method is the fact that it allows for an in vitro selection of (a) cell(s), (a) tissue(s) or (a) cell culture with susceptibility to an HSP90 inhibitor/responsive to treatment with an HSP90 inhibitor. As documented herein, genomically annotated NSCLC cell lines are used that are representative of the genetic diversity, the transcriptional profile and the phenotypic properties of primary NSCLC (non-small cell lung cancer) tumors. It is shown herein by integrated genomic profiling on a global scale that the genomes of non-small cell king cancer (NSCLC) cell lines are highly representative of several primary NSCLC tumors isolated by surgery from patients in gene copy number, oncogene mutation and gene expression space. Thus, using said NSCLC cell line collection in context of the present invention may avoid animal tests or voluntary tests with cancer patients; at the same time use of said cell line collection allows, in contrast to methods known in the art, the reliable identification of KRAS mutations as markers for susceptibility to HSP90 inhibitors. It is envisaged that other cell line collection may be used herein as long as the highly representative of primary NSCLC tumors, in particular in respect of gene copy number, oncogene mutation and gene expression space. Also computational approaches described herein and used in the appended example, may be used in context of the present invention for the evaluation of experimental data and/or identification of predictors/markers of (a) (tumor) cell(s), (a) (tumor) tissue(s), or (a) (tumor) cell culture for susceptibility to an HSP90 inhibitor. Such computational approaches are known in the art and comprise, inter alia, K-nearest neighbour and statistical tests, such as Fisher's exact test. A person skilled in the art knows how to use these computational approaches in context of the present invention. A skilled person will also be aware of further computational approaches to be used in accordance with the present invention and may adapt said approaches based on his general knowledge. The description herein below and the appended examples provide for a specific selection of cell lines or combination of cell lines which can be used as experimental set in the assessment of markers for drug susceptibility.

A systematical annotation of the genomes of a large panel of NSCLC cell lines was performed in order to determine whether such a collection reflects the genetic diversity of primary NSCLC tumors. As described in the appended example, the phenotypic validity of this collection was demonstrated, thus confirming that the genomes of non-small cell lung cancer (NSCLC) cell lines are highly representative of primary NSCLC tumors. Said cell lines may, accordingly, be used in analysis of drug activity as a function of genomic lesions in a systematic fashion as described herein.

Using the systematic similarity profiling described in the appended example, EGFR mutations were confirmed to predict sensitivity to EGFR inhibitors (erlotinib, PD168393, vandetanib) (Arao et al., 2004; Lynch et al., 2004; Paez et al., 2004; Pao et al., 2004; Sos et al., 2008) which is in accordance with prior art observations. A high activity of EGFR inhibitors in EGFR-mutant NSCLC cell lines has been described in the prior art (McDermott et al., 2007; Paez et al., 2004; Tracy et al., 2004). As mentioned, these findings of the prior art have been confirmed herein using an unbiased computational approach employing systematic global measurements of genetic lesions. These findings described in the prior art in respect of the identification of EGFR mutations as predictors/markers for susceptibility to EGFR inhibitors have been confirmed in the present invention, thus demonstrating the overall functional biological validity of the computational approach used in the appended example for the identification of KRAS mutations as predictors/markers for susceptibility to HSP90 inhibitors. For example, EGFR-mutations were identified as markers/predictors of susceptibility to EGFR inhibition (p<0.0001) using the systematic cell-based compound screening followed by computational prediction of sensitivity based on lesion profiles. However, not only EGFR-mutations were identified and confirmed as predictors/markers for susceptibility to EGFR inhibitors but also, and unexpectedly, KRAS mutations as predictors/markers for susceptibility to an HSP90 inhibitor. It is to be understood that in-depth preclinical analysis of activity of cancer therapeutics in tumor cells requires both thorough genomic analysis of a large cell line collection of a single tumor entity and high-throughput cell line profiling, followed by genomic prediction of compound activity as described and demonstrated herein.

The accuracy of the identification of KRAS mutations as predictors/markers for susceptibility to an HSP90 inhibitor may further be supported by structural modeling of compound binding to HSP90 as shown in the appended example in respect of vandatinib/erlotinib binding in the EGFR kinase domain. Vandatinib/Erlotinib were also formally confirmed as EGFR inhibitors by demonstrating the lack of activity of this compound in Ba/F3 cells expressing the T790M resistance allele. EGFR mutations were also identified herein as predictors/markers for susceptibility to the SRC/ABL inhibitor dasatinib. Without being bound by theory this finding formally proves that mutant EGFR is an actual target of dasatinib (Song et al., 2006).

The terms “susceptibility to an HSP90 inhibitor” and “responsiveness to treatment with an HSP90 inhibitor” are used interchangeably in context of the present invention. Any explanations given herein in respect to “susceptibility to an HSP90 inhibitor” also apply to “responsiveness to treatment with an HSP90 inhibitor”, mutatis mutandis, and vice versa.

Technical means and methods for determining the susceptibility to drugs are known in the art. Such methods comprise, inter alia, cell or tissue culture experiments. Through less preferred, it is also envisaged herein that two or more different HSP90 inhibitor(s) (i.e. HSP90 having different chemical formulae, optionally non-structurally related HSP90 inhibitors) may be tested simultaneously. However, it is preferred herein that only one HSP90 inhibitor(s) is tested at one time. Preferred HSP90 inhibitors to be used and tested in the present invention are described herein below.

The selection methods or method for determining the responsiveness to treatment with an HSP90 inhibitor provided herein may comprise a contacting step/exposing step which is explained in more detail herein below. The term “treatment with an HSP90 inhibitor” as defined herein implies contacting/exposing, for example, a mammalian tumor cell or cancer cell with/to said inhibitor. Of course, also other cells described herein below may be treated with an HSP90 inhibitor.

These above-mentioned methods may also comprise an evaluation/determination step, which may, for example, include determining the viability of the cell(s), tissue(s) or cell culture(s) contacted with/exposed to an HSP90 inhibitor or (a) mammalian cell(s) treated with an HSP90 inhibitor. For example, (a) cell(s), (a) tissue(s) or (a) cell culture(s) described herein above may show decreased viability upon contacting/exposing/treating with an HSP90 inhibitor. Preferably, the cell(s), tissue(s) or cell culture(s) may show an at least 10%, 20%, 30%, 40%, 50%, 60%, 70%, 80%, and most preferably, 90% reduction in viability compared to control cell(s), tissue(s) or cell culture(s) not contacted/exposed/treated with an HSP90 inhibitor. Preferably, the control cell(s), (a) tissue(s) or (a) cell culture(s) will be identical to the cell(s), (a) tissue(s) or (a) cell culture(s) to be tested as described herein with the only exception that the control (s), (a) tissue(s) or (a) cell culture(s) are not contacted with/exposed to the HSP90 inhibitor.

Thus (a) cell(s), (a) tissue(s) or (a) cell culture(s) contacted/exposed/treated with an HSP90 inhibitor and showing, for example, a decreased proliferation as described herein above, can be considered as being susceptible to an HSP90 inhibitor. Correspondingly, (a) mammalian cell(s) treated with an HSP90 inhibitor showing such a decreased proliferation can be considered as responsive to treatment with an HSP90 inhibitor. The step of determining the presence of at least one mutation in the KRAS gene and corresponding mutations in the KRAS gene are described herein below. As already mentioned above, it has been surprisingly found in context of this invention that the presence of such a mutation in the KRAS gene is indicative of whether (a) cell(s), (a) tissue(s) or (a) cell culture(s) contacted/treated with or exposed to an HSP90 inhibitor is susceptible to an HSP90 inhibitor or responsive to treatment with an HSP90 inhibitor. A reduction in viability may, for example, be reflected in a decreased proliferation, such as 10%, 20%, 30%, 40%, 50%, 60%, 70%, 80%, and most preferably. 90% reduction in proliferation compared to control cell(s), tissue(s) or cell culture(s) not contacted/exposed/treated with an HSP90 inhibitor. The decreased proliferation may be quantitated, for example, by measuring the total cell volume, tissue volume or cell culture volume using standard techniques.

The difference in proliferation between contacted/exposed/treated tissue(s) or cell culture(s) and corresponding controls as defined herein may, for example, be evaluated/determined by measuring the volume of the cell(s), tissue(s) or cell culture(s) taking advantage of standard techniques. Said evaluation/determination may be performed in various points in time, for example, 15 minutes, 30 minutes, 60 minutes, 2 hours, 5 hours, 18 hours, 24 hours, 2 days, 3 days, 4 days, 5 days, six days and/or seven days after contacting/treating, said cell(s), tissue(s) or cell culture(s) with an HSP90 inhibitor or exposing said cell(s), tissues) or cell culture(s) to an HSP90 inhibitor. It is envisaged herein that said evaluation/determination may be performed repeatedly, for example, at 15 minutes, 30 minutes and 60 minutes after said contacting/exposing/treating. It is of note that said cell(s), tissue(s) or cell culture(s) may be contacted/treated not only once with said HSP90 inhibitor or exposed to said HSP90 inhibitor but several times (e.g. 2 times, 3 times, 5 times, 10 times or 20 times) under various conditions (e.g. same concentration of inhibitor, different concentration of inhibitor, inhibitor comprised in a composition with different stabilizers, diluents, and/or carriers and the like). Accordingly, said optionally repeated evaluation/determination may be performed after the final contacting/treating with or exposing to said HSP90 inhibitor or in between said above-mentioned various contacting/exposing/treating steps. The explanations given herein above in respect of the exemplary determination/evaluation step, comprising determining the proliferation of the cell(s), tissue(s) or cell culture(s) contacted with/exposed to an HSP90 inhibitor apply to other determination/evaluation steps described herein (e.g. determining the level of HSP90 expression) and further determination/evaluation steps a person skilled in the art will be aware of such as assays that quantitate the induction of apoptotic cell death, senescence or any other cell biology phenotype that is associated with decreased viability or proliferation of tumor cells.

These selection methods or method for determining the responsiveness to treatment with an HSP90 inhibitor may also comprise determining the level of HSP90 activity, wherein the activity of HSP90 is indicative whether the cell(s), tissue(s) or cell culture(s) is (are) susceptible to an HSP90 inhibitor or is responsive to treatment with an HSP90 inhibitor. As described herein below in context of the determination of the activity of KRAS and optionally of EGFR and/or BRAF, the term “activity” used herein comprises, for example, determining the enzymatic activity at the protein level and/or the determination of the expression level (e.g. mRNA or protein). Methods for determining the activity as defined herein are well known in the art and also described herein below.

The term “HSP90” (“Heat shock protein 90”) as used in context of this invention refers to a molecular chaperone which is essential for viability in eukaryotic organisms. Heat shock protein 90 is a molecular chaperone that is responsible for correcting misfolded proteins and therefore exerts an important molecular control function in healthy cells. In tumor cells, it was found that mutationally activated oncogenes such as BRAF or EGFR depend on proper maturation by HSP90. Despite the fact that HSP90 has as its original function the protection of cells, tumor cells exploit these capabilities for stabilizing the oncogenes that are driving oncogenic transformation.

The nucleic acid sequence of various transcript variants of human HSP90 and the corresponding amino acid sequence of HSP90 isoforms are shown in SEQ ID NOs: 137, 139, 141, 143 and 145, and SEQ ID NOs: 138, 140, 142, 144 and 146, respectively. It is speculated in the art that the nucleic acid sequence encoding HSP90AA2 (SEQ ID NO: 145) may be a pseudogene. Generally, the term HSP90 used herein refers to any amino acid sequence having (partial) HSP90 activity as described herein and nucleic acid sequence(s) encoding such (an) amino acid sequence(s).

The nucleic acid sequences of HSP90 of other mammalian or non-mammalian species (in particular rat, chimpanzee, macaque, C. elegans, Drosophila melanogaster) than the herein provided sequences for human HSP90 can be identified by the skilled person using methods known in the art, e.g. by nucleic acid sequencing or using hybridization assays or by using alignments, either manually or by using computer programs such as those mentioned herein below in connection with the definition of the term “hybridization” and degrees of homology. In one embodiment, the nucleic acid sequence encoding for orthologs of human HSP90 is at least 40% homologous to the nucleic acid sequences as shown in SEQ ID NOs: 137, 139, 141, 143 and 145. More preferably, the nucleic acid sequence encoding for orthologs of human HSP90 is at least 45%, 50%, 55%, 60%, 65%, 70%, 75%, 80%, 85%, 90%, 91%, 92%, 93%, 94%, 95%, 96%, 97% or 98% homologous to the nucleic acid sequence as shown in SEQ ID NOs. 137, 139, 141, 143 and 145, wherein the higher values are preferred. Most preferably, the nucleic acid sequence encoding for orthologs of HSP90 is at least 99% homologous to the nucleic acid sequence as shown in SEQ ID NOs. 137, 139, 141, 143 and 145.

Hybridization assays for the characterization of orthologs of known nucleic acid sequences/promoters are well known in the art; see e.g. Sambrook, Russell “Molecular Cloning, A Laboratory Manual”, Cold Spring Harbor Laboratory, N.Y. (2001): Ausubel, “Current Protocols in Molecular Biology”, Green Publishing Associates and Wiley Interscience, N.Y. (1989). The term “hybridization” or “hybridizes” as used herein may relate to hybridizations under stringent or non-stringent conditions. If not further specified, the conditions are preferably non-stringent. Said hybridization conditions may be established according to conventional protocols described, e.g. in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

In accordance with the present invention, the terms “homology” or “percent homology” or “identical” or “percent identity” or “percentage identity” or “sequence identity” in the context of two or more nucleic acid sequences refers to two or more sequences or subsequences that are the same, or that have a specified percentage of nucleotides that are the same (preferably at least 40% identity, more preferably at least 45%, 50%, 55%, 60%, 65%, 70%, 75%, 80%, 85%, 90%, 91%, 92%, 93%, 94%, 95%, 96%, 97% or 98% identity, most preferably at least 99% identity), when compared and aligned for maximum correspondence over a window of comparison, or over a designated region as measured using a sequence comparison algorithm as known in the art, or by manual alignment and visual inspection. Sequences having, for example, 75% to 90% or greater sequence identity may be considered to be substantially identical. Such a definition also applies to the complement of a test sequence. Preferably the described identity exists over a region that is at least about 15 to 25 nucleotides in length, more preferably, over a region that is at least about 50 to 100 nucleotides in length and most preferably, over a region that is at least about 800 to 1200 nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. The BLOSUM62 scoring matrix (Henikoff (1989) PNAS 89:10915) uses alignments (B) of 50, expectation (E) of 10, M=5, N=4 and a comparison of both strands.

In order to determine whether a nucleotide residue in a nucleic acid sequence corresponds to a certain position in the nucleotide sequence of e.g. SEQ ID NOs: 137, 139, 141, 143 and 145, the skilled person can use means and methods well-known in the art, e.g., alignments, either manually or by using computer programs such as those mentioned herein. For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches, which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

The term “HSP90 inhibitor” means accordingly in this context a compound capable of inhibiting the expression and/or activity of “HSP90” defined herein above. An HSP90 inhibitor may, for example, interfere with transcription of a HSP90 gene, processing (e.g. splicing, export from the nucleus and the like) of the gene product (e.g. unspliced or partially spliced mRNA) and/or translation of the gene product (e.g. mature mRNA). The HSP90 inhibitor may also interfere with further modification (like glycosylation) of the polypeptide/protein encoded by the HSP90 gene and thus completely or partially inhibit the activity of the HSP90 protein as described herein above.

Furthermore, the HSP90 inhibitor may interfere with interactions of the HSP90 protein with other proteins, in particular with mutant proteins to be stabilized by HSP90 activity.

Also siRNAs/RNAis, antisense molecules and ribozymes directed against nucleic acid molecules encoding HSP90 are envisaged as (an) HSP90 inhibitor(s) for the use and the method of the present invention. The above-mentioned antagonist/inhibitor of HSP90 may also be a co-suppressive nucleic acid.

An siRNA approach is, for example, disclosed in Elbashir ((2001), Nature 411, 494-498)). It is also envisaged in accordance with this invention that for example short hairpin RNAs (shRNAs) are employed in accordance with this invention as pharmaceutical composition. The shRNA approach for gene silencing is well known in the art and may comprise the use of st (small temporal) RNAs; see, inter alia, Paddison (2002) Genes Dev. 16, 948-958.

As mentioned above, approaches for gene silencing are known in the art and comprise “RNA”-approaches like RNAi (iRNA) or siRNA. Successful use of such approaches has been shown in Paddison (2002) loc. cit., Elbashir (2002) Methods 26, 199-213; Novina (2002) Mat. Med, Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002) loc. cit.; Miyagishi (2002) loc. cit. or Brummelkamp (2002) loc. cit.

Further HSP90 inhibitors are well known in the art and are, for example, described in (Sharp and Workman, 2006; Solit and Rosen, 2006). However, use of HSP90 inhibitors in accordance with the present invention is not limited to known HSP90 inhibitors. Accordingly, also yet unknown HSP90 inhibitors may be used in accordance with the present invention. Such inhibitors may be identified by the methods described and provided herein and methods known in the art, like high-throughput screening using biochemical assays for inhibition of HSP90 (Sharp and Workman. 2006; Solit and Rosen, 2006). Based on his general knowledge a person skilled in the art is easily in the position to identify inhibitors or verify the inhibiting activity of compounds suspected of being HSP90 inhibitors. These tests may be employed on cell(s) or cell culture(s) described in the appended example, but also further cell(s)/tissue(s)/cell culture(s) may be used, such as cell(s)/tissues)/cell culture(s) derived from biopsies.

In preferred embodiments of the present invention the HSP90 inhibitor is geldanamycin or a derivative thereof. Geldanamycin (IUPAC name ([18S-(4E,5Z,8R*,9R*,10E,12R*,13S*,14R*,16S*)]-9-[(aminocarbonyl)oxy]-13-hydroxy-8,14,19-trimetoxy-4,10,12,16-tetramethyl-2-azabicyclo[16.3.1.]docosa-4,6,10,18,21-pentan-3,20,22-trion) is a benzoquinone ansamycin antibiotic which may be produced by the bacterium Streptomyces hygroscopicus. Geldanamycin binds specifically to HSP90 (Heat Shock Protein 90) and alters its function. While Hsp90 generally stabilizes folding of proteins and, in particular in tumor cells, folding of overexpressed/mutant proteins such as v-Src, Bcr-Abl and p53, the Hsp90 inhibitor Geldanamycin induces degradation of such proteins.

The respective formula of geldanamycin is given herein below:

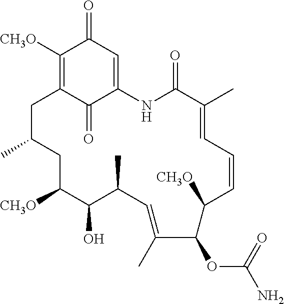

Even though geldanamycin is a potent antitumor agent, the use of geldanamycin also shows some negative side-effects (e.g. hepatotoxicity) which led to the development of geldanamycin analogues/derivatives, in particular analogues/derivatives containing a derivatisation at the 17 position. Without being bound by theory, modification at the 17 position of geldanamycin may lead to decreases hepatotoxicity. Accordingly geldanamycin analogues/derivatives which are modified at the 17 position, such as 17-AAG (17-N-Allylamino-17-demethoxygeldanamycin), are preferred in context of the present invention. Also preferred herein are geldanamycin derivatives to be used in accordance with the present invention which are water-soluble or which can be dissolved in water completely (at least 90%, more preferably 95%, 96%, 97%, 98% and most preferably 99%).

17-AAG ([(3S,5S,6R,7S,8E,10R,11S,12E,14E)-21-(allylamino)-6-hydroxy-5,11-dimethoxy-3,7,9,15-tetramethyl-16,20,22-trioxo-17-azabicyclo[16.3.1]docosa-8,12,14,18,21-pentaen-10-yl]carbamate) is as mentioned above a preferred derivative of geldanamycin. 17-AAG is commercially available under the trade name “Tanespimycin” (also known as KOS-953) for example from Kosan Biosciences Incorporated (Acquired by Bristol-Myers Squibb Company). Tanespimycin is presently studied in phase II clinical trials for multiple myeloma and breast cancer and is usually administered intravenously.

The respective formula of 17-AAG is given herein below:

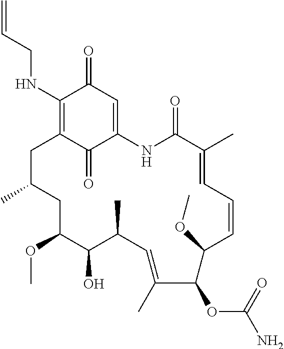

Preferred geldanamycin-derivative (HSP90 inhibitor) to be used in context of the present invention are IPI-504 (also known as retaspimycin or Medi-561; infinity Pharmaceuticals (MedImmune/Astra Zeneca)). Clinical trials on the use of IPI-504 (which is usually administered intravenously) in the treatment of non-small cell lung cancer (NSCLC) and breast cancer are performed. Also alvespimycin hydrochloride (Kosan Biosciences Incorporated Acquired By: Bristol-Myers Squibb Company) is a highly potent, water-soluble and orally active derivative of geldanamycin preferably used in context of the present invention.

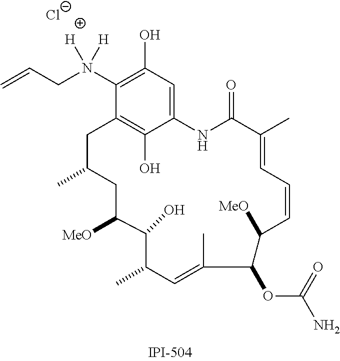

Further geldanamycin-derivatives to be used in accordance with the present invention are, inter alia, ABI010 (Abraxis BioScience, Inc. (ABM) which is a nab-17AAG being developed using nab technology and Retaspimycin hydrochloride, a highly soluble hydroquinone hydrochloride salt of 17-AAG (Infinity Pharmaceuticals Inc (INFI)).

Other, exemplary derivatives of geldanamycin which may be used in context of the present invention are 17-DMAG (17-NN-Dimethyl Ethylene Diamine-Geldanamycin), and CNF1010 (an ansamycin-based semi-synthetic analogue of geldanamycin (Biogen Idec Inc (BIIB)/Con form a). The anti-tumor activity of 17-DMAG is, for example, described in Hollingshead (2005) Cancer Chemother Pharmacol 56, 115-125. The respective formula of 17-DMAG (also known as alvespimycin) is indicated herein below:

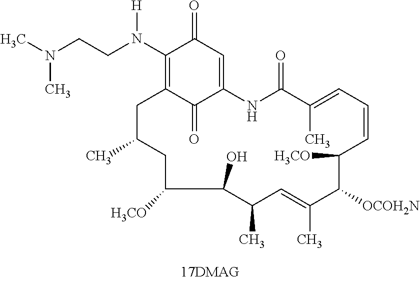

A further geldanamycin-like HSP90 inhibitor to be used in context of the present invention is IPI-493 (orally available inhibitor of HSP90 (Infinity Pharmaceuticals (Medimmune/AstraZeneca)). Clinical phase I trials for IPI-493 are scheduled for 2010.

Also the use of non-geldanamycin-like HSP90 inhibitors (i.e. HSP90 inhibitors which are not geldanamycin-derivatives as described above) is envisaged in context of the present invention. Non-geldanamycin-like HSP90 inhibitors to be used herein are isoaxoles, in particular 4,5-diaryl-Isoaxol HSP90 inhibitors, for example NVP-AUY922. Isoaxole compounds are known in the art as inhibitors of heat shock proteins; see WO 2004/072051 and WO 2008/104595. NVP-AUY922 (Novartis AG (NVS)), also known as VER-52296 (Vernalis) or AUY922 is known as a potent HSP90 inhibitor and its antitumor activity (e.g. in breast cancer models or in a human colon cancer xenograft model) has also been described; see Brough (2008) J. Med. Chem (2008) 51(2), 196-218 and Jensen (2008) Breast Cancer Res 10:R33. Recently, NVP-AUY922 has entered clinical phase I breast cancer trials.

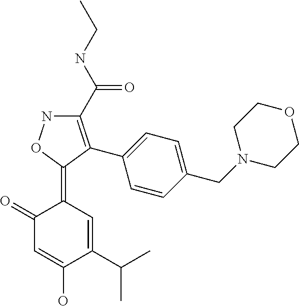

NVP-AUY922 can be administered intravenously but also oral administration is envisaged.

Further, non-limiting examples of HSP90 inhibitors which may be used in accordance with the present invention are XL888 (orally available ATP competitive inhibitor (Exelexis))ARQ250RP (ArQule Inc (ARQL), AT13387 (a non-ansamycin inhibitor for oral and intravenous administration; Astex Therapeutics (Private)), BHI001 (a water soluble polyketide; Biotica Technology Limited (Private)), BIIB021(CNF2024) (an orally bioavailable HSP90 inhibitor presently in clinical phase II studies for gastrointestinal stromal tumors (GIST); Biogen Idec Inc (BIIB)/Conforma), HBP-347 (orally available, contains hypericin as an active ingredient and is being tested in clinical phase I/II studies for brain and haematological cancer; Hy BioPharma Inc. (Private)), KW-2478 (intravenous administered and presently in clinical phase I studies for B-cell malignancies; Kirin/Kyowa Hakko Kogyo Co., Ltd), MPC3100 (Myriad Genetics Inc (MYGN)), MPC3160 (orally administered, in clinical phase I studies; Myriad Genetics Inc.), SAR567530 (Sanofi-Aventis (SNY)), SNX-5542 (orally administered, in clinical phase I studies for breast cancer; prodrug; a, water soluble, orally active small molecule; active form: SNX-2122; Pfizer/Serenex, Inc. (Private)), SRN005 (KeyNeurotek AG (Private)) and STA-9090 (intravenous administration; an injectable, small molecule unrelated to geldanamycin or its family of related compounds; in clinical phase I trials for haematological cancer; Synta Pharmaceuticals Corp. (SNTA)). The use of antibodies as HSP90 inhibitors is also envisaged in context of the present invention. For example, the HSP90 inhibitor “Mycograb” may be used which is a human genetically recombinant antibody and binds to the immunodominant fungal antigen heat shock protein 90 (Novartis AG (NVS)).

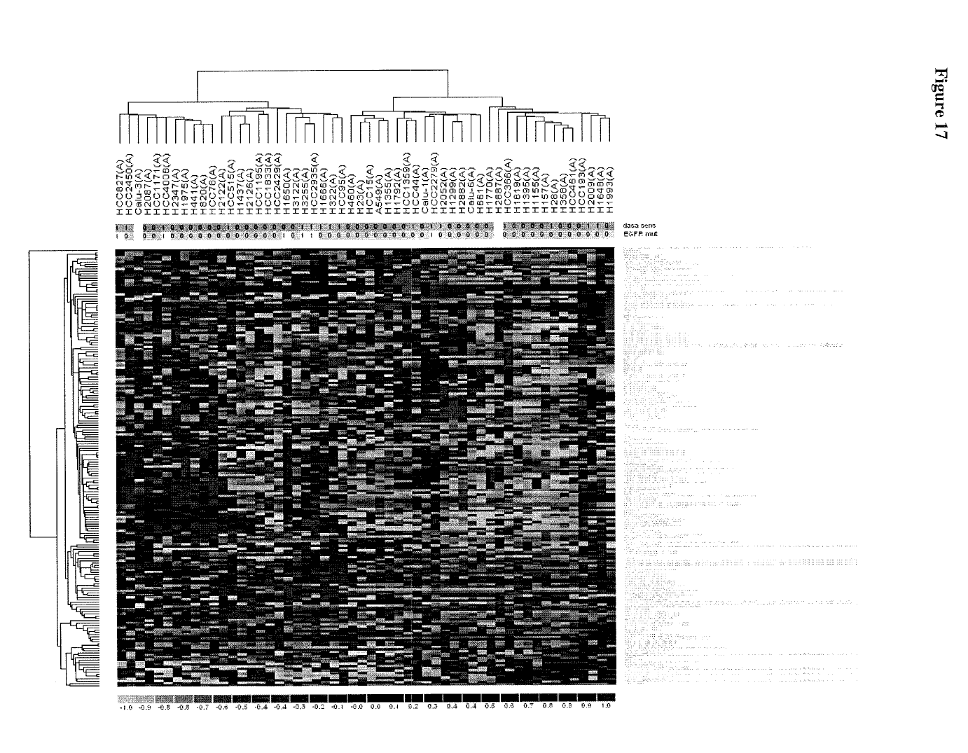

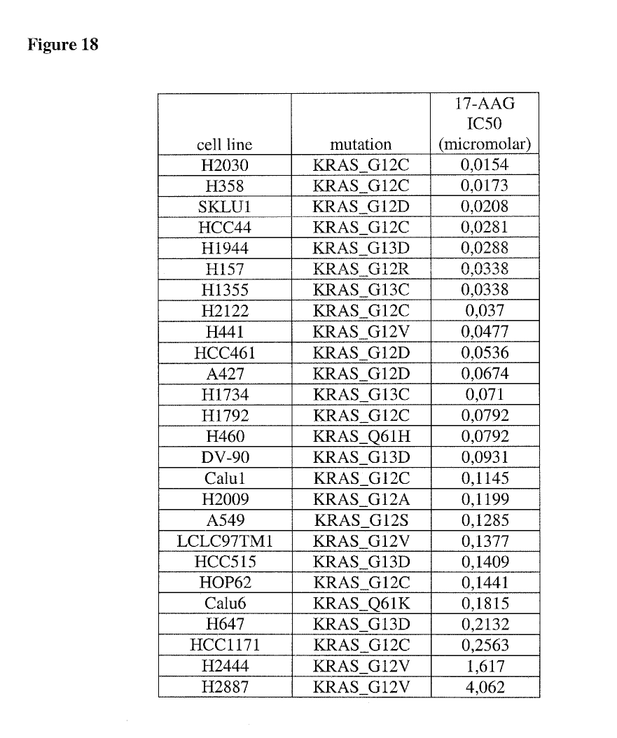

As described herein above, it was surprisingly found herein that a mutation in the KRAS gene is indicative for susceptibility to an HSP90 inhibitor or responsiveness to the treatment with an HSP90 inhibitor. Mutations in the KRAS gene are well known in the art and for example described in the COSMIC data base (www.sanger.ac.uk/genetics/CGP/cosmic/). Preferred mutations in the KRAS gene are KRAS_G12C, KRAS_G12R, KRAS_G12D, KRAS_G12A, KRAS_G12S, KRAS_G12V, KRAS_G13D, KRAS_G13S, KRAS_G13C, KRAS_G13V, KRAS_Q61H, KRAS_Q61R. KRAS_Q61P, KRAS_Q61L, KRAS_Q61K, KRAS_Q61E, KRAS_A59T and KRAS_G12F. These mutations and corresponding cell lines carrying such a mutation in the KRAS gene/characterized by such a mutation in the KRAS gene are also shown in FIG. 18. The nucleotide sequences and amino acid sequences of the above-mentioned. KRAS mutations are depicted in SEQ ID NOs: 3 to 78, whereas the nucleic acid sequences of a “wild-type” (i.e. carrying no KRAS mutation) KRAS nucleotide sequences are depicted in SEQ ID NOs:1 and 157 (reflecting two isoforms of KRAS gene). With this information, the person skilled in the art can easily deduce the above-mentioned mutations in the KRAS gene product. Amino acid sequences (of two isoforms of KRAS) are provided in SEQ ID NOs: 2 and 158. Accordingly, the above-mentioned mutations can also be deduced from the two wild-type sequences provided in SEQ ID NOs: 2 and 158. In particular, activating mutations in the KRAS gene are of importance in the embodiments provided herein.

In one embodiment of the present invention, not only the presence of (a) mutation(s) in the KRAS gene (in particular an activating mutation) is determined, but also, as a further option, a mutation in the EGFR and/or the BRAF gene is additionally determined. The mutation in the EGFR gene to be determined may, for example, be EGFR_D770_N771>AGG; EGFR_D770_N771insG; EGFR_D770_N771insG; EGFR_D770N771insN; EGFR_E709A; EGFR_E709G; EGFR_E709H; EGFR_E709K; EGFR_E709V; EGFR_E746_A750del; EGFR_E746_A750del, T751A; EGFR_E746_A750del, V ins; EGFR_E746_T751del, I ins; EGFR_E746_T751del, S752A; EGFR_E746_T751del, S752D; EGFR_E746 T751del, V ins; EGFR_G719A; EGFR_G719C; EGFR_G719S; EGFR_H773_V774insH; EGFR_T773_V774insNPH; EGFR_F773_V774insPH; EGFR_H773>NPY; EGFR_L747_E749del; EGFR_L747E749del, A750P; EGFR_L747S752del; EGFR_L747 S752del, P753S; EGFR_L747 S752del, Q ins; EGFR_L747_T750del, P ins; EGFR_L747_T751 del; EGFR_L858R; EGFR_L861Q; EGFR_M766_A767insAI; EGFR_P772_H773insV; EGFR_S752_I759del; EGFR_S768I; EGFR_T790M; EGFR_V769_D770insASV; EGFR_V769_D770insASV; and EGFR_V774_C775insHV. It is of note that mutations in EGFR include complex mutations where amino acids are inserted or deleted. In some cases, such event occurs in conjunction with a substitution mutation. In the case of deletions, the amino acids from before the second underscore (“_”) in the mutation formula through the one following the underscore are deleted (e.g., “EGFR_E746_A750del” refers to a mutant version of EGFR where the amino acids 746 through 750 are deleted). In the case of insertions, the insertion occurs in between the two amino acids separated by the underscore (e.g., “EGFR_P772_H773insV” refers to a mutant version of EGFR where the amino acid V is inserted between P772 and H773 of the wild-type sequence). If a substitution or insertion follows an insertion or deletion, the substituted or inserted amino acid is preceded by a comma (“,”) following the description of the insertion or deletion. E.g., the mutation “EGFR_E746_T751del, V ins” refers to a mutant version of EGFR where amino acids E746 through T751 are deleted, followed immediately by an insertion of a V. Alternatively, “EGFR_E746_A750del, T1751A” refers to a mutant version of EGFR where the deletion of amino acids 746 through 750 is followed by a substitution of T751 with A.

Nucleic acid sequences of a “wild-type” (i.e. carrying no EGFR mutation) EGFR nucleotide sequences are depicted in SEQ ID NOs: 149, 151, 153 and 155 (reflecting four isoforms of EGFR). With this information, the person skilled in the art can easily deduce the above-mentioned mutations in the EGFR gene product. Amino acid sequences of “wild-type” EGFR are provided in SEQ ID NOs: 150, 152, 154 and 156. Accordingly, the above-mentioned mutations can also be deduced from the wild-type sequences provided in SEQ ID NOs: 150, 152, 154 and 156.

Preferred mutations in the BRAF gene are BRAF_D594G, BRAF_D594V, BRAF_F468C, BRAF_F595L, BRAF_G464E, BRAF_G464R, BRAF_G464V, BRAF_G466A, BRAF_G466E, BRAF_G466R, BRAF_G466V, BRAF_G469A, BRAF_G469E, BRAF_G469R, BRAF_G469R, BRAF_G469S, BRAF_G469V, BRAF_G596R, BRAF_K601E. BRAF_K601N, BRAF_L597Q BRAF_L597R, BRAF_L597S, BRAF_L597V, BRAF_T599I, BRAF_V600E, BRAF_V600K, BRAF_V600L, BRAF_V600R

The nucleotide sequences and amino acid sequences of the above-mentioned BRAF mutations are depicted in SEQ ID NOs: 79 to 136, whereas the nucleic acid sequence of a “wild-type” (i.e. carrying no BRAF mutation) BRAF nucleotide sequence is depicted in SEQ ID NO:147. With this information, the person skilled in the art can easily deduce the above-mentioned mutations in the BRAF gene product. An amino acid sequence of BRAF is provided in SEQ ID NO: 148. Accordingly, the above-mentioned mutations can also be deduced from the wild-type sequence provided in SEQ ID NO: 148.

The one (or three) letter code used for annotating the above-mentioned mutations in the amino acid sequences of the (mutated) KRAS gene product, (mutated) EGFR gene product and (mutated) BRAF gene product is well known in the art and may be deduced from standard text books (such as “The Cell”, Garland Publishing, Inc. third edition). A comprehensive list of the specific list of amino acids and their respective abbreviations using the one (or three) letter code is given herein below:

| | | |
|---|---|---|
| Alanine | A | Ala |
| Cysteine | C | Cys |
| Aspartic acid | D | Asp |
| Glutamic acid | E | Glu |
| Phenylalanine | F | Phe |
| Glycine | G | Gly |
| Histidine | H | His |
| Isoleucine | I | Ile |
| Lysine | K | Lys |
| Leucine | L | Leu |
| Methionine | M | Met |
| Asparagine | N | Asn |
| Proline | P | Pro |
| Glutamine | Q | Gln |
| Arginine | R | Arg |
| Serine | S | Ser |
| Threonine | T | Thr |
| Valine | V | Val |
| Tryptophan | W | Trp |
| Tyrosine | Y | Tyr |

As mentioned above, (a) tumor cell(s)/tumor(s) with (a) mutation(s) in the EGFR and/or the BRAF gene is (are) sensitive to treatment with HSP90 inhibitors. Therefore, it is envisaged that (a) tumor cell(s)/tumor(s) with (a) mutation(s) in the KRAS gene and, optionally, in the EGFR and/or the BRAF gene might be particularly sensitive to treatment with HSP90 inhibitors. Therefore, (a) cell(s), (a) tissue(s) or (a) cell culture selected in accordance with the present method with at least one mutation in the KRAS gene and in the EGFR and/or the BRAF gene might be particularly susceptible to (an) HSP90 inhibitor(s). Accordingly, treatment of patients with an HSP90 inhibitor (the patients suffering from a cancer characterized by the presence of at least one mutation in the KRAS gene and in the EGFR and/or the BRAF gene may be particularly successful in respect of, for example, prognosis or survival rate.

The determination of the presence of other markers for susceptibility to HSP90 inhibitors in addition to (a) mutation(s) in the KRAS gene and (as a further option), additionally, in the EGFR and/or the BRAF gene is also envisaged herein. Of course, also the presence of markers for susceptibility to other compounds/drugs (e.g. EGFR inhibitors and the like) may be determined in accordance with the present methods. This may be of value in selecting (a) cell(s), (a) tissue(s) or (a) cell culture or in the identification of patients/responders which are not only susceptible/sensitive to (an) HSP90 inhibitor(s) but also to other compounds/drugs (e.g. (an) EGFR inhibitor(s)). These cell(s)/tissue(s)/cell culture(s)/patient(s)/responder(s) may, for example, be subject to co-therapy/co-treatment with an HSP90 inhibitor and a further compound/drug (e.g. (an) EGFR inhibitor(s)).

Alternatively, (a) cell(s), (a) tissue(s) or (a) cell culture may be selected or patients/responders be identified which are characterized by the presence only of (an) mutations(s) in the KRAS gene but not in the EGFR and/or the BRAF gene and/or optionally further genes indicated herein above. For example, patients suffering from cancer characterized by the presence of at least one mutation in the KRAS gene and (a) mutation(s) in a further gene (e.g. EGFR) may only be treated with an HSP90 inhibitor but not in co-therapy with an EGFR and an HSP90 inhibitor if the patients are known to be resistant to such an EGFR inhibitor. Of course, co-therapy/combination therapy to be used in context of the present invention may also comprise radiation therapy, conventional chemotherapy and the like.

The mutations in the KRAS gene, the EGFR and/or the BRAF gene and optionally further genes can be detected by methods known in the art. Such methods are, for example described in (Papadopoulos et al., 2006; Shendure et al., 2004). A person skilled in the art is in the position to adapt the methods for detecting mutations in genes described in the above-mentioned documents to the KRAS-, EGFR- and/or the BRAF-mutations described herein and further mutations in these genes known in the art. A person skilled in the art will readily understand that also mutations in said genes not described herein but known in the art or mutations yet to be identified may also be used in the context of the present invention. Exemplary, non-limiting methods to be used in the detection of mutations in the KRAS gene, and optionally in the EGFR and/or the BRAF gene are methods for sequencing of nucleic acids (e.g. Sanger di-deoxy sequencing), “next generation” methods, single molecule sequencing, methods enabling detection of variant alleles/mutations, such as Real-time PCR, PCR-RFLP assay (see Cancer Research 59 (1999), 5169-5175), mass-spectrometric genotyping (e.g. MALDI-TOF), enzymatic methods and SSPC (single strand conformation polymorphism analysis; see Pathol Int (1996) 46, 801-804).

In particular, such methods may include enzymatic amplification of DNA or cDNA fragments using oligonucleotides specifically hybridizing to exonic or intronic parts of the KRAS gene by per. Given that most (>95%) of all mutations in the KRAS gene occur in exons 2 or three, such amplifications may be carried out in two reactions when employing genomic DNA or even in only a single reaction when employing cDNA. The resulting PCR products may be subjected to either conventional Sanger-based dideoxy nucleotide sequencing methods or employing novel parallel sequencing methods (“next generation sequencing”) such as those marketed by Roche (454 technology), Illumina (Solexa technology) or ABI (Solid technology). Mutations may be identified from sequence reads by comparison with publicly available gene sequence data bases. Alternatively, mutations may be identified by allele-specific incorporation of probes that can either be detected using enzymatic detection reactions, fluorescence, mass spectrometry or others; see Vogeser (2007) Dtsch Arztebl 104 (31-32), A2194-200.

Paraffin-embedded clinical material may be used in the detection of KRAS mutations. Detection may comprise a histolopathology review of the sample to be tested to ensure tumour tissue is present. A particular assay to be used in the detection method is the DxS Ltd Therascreen™ KRAS Mutation Testing kit, which is designed to detect the 7 most common activating mutations of the KRAS gene. Another commercially available Kit to be used in the detection method is the DxS K-RAS Mutation Test Kit. According to the manufacturer, this kit covers 98.5% of the mutations registered in the COSMIC Release 35 within the large intestine, i.e. the following K-RAS mutations: 12Asp (GGT>GAT), 12Val (GGT>GTT), 12Cys (GGT>TGT), 12Ser (GGT>AGT), 12Ala (GGT>GCT), 12Arg (GGT>CGT) and 13Asp (GGC>GAC). These mutations are also described herein as activating mutations in the KRAS gene which are indicative for susceptibility to an HSP90 inhibitor or responsiveness to the treatment with an HSP90 inhibitor. The correlation of the designations of the K-RAS mutations used in the above kit and used herein is given in the table below:

| | |
|---|---|
| 12Asp (GGT > GAT) | KRAS_G12D |
| 12Val (GGT > GTT) | KRAS_G12V |
| 12Cys (GGT > TGT) | KRAS_G12C |
| 12Ser (GGT > AGT) | KRAS_G12S |
| 12Ala (GGT > GCT) | KRAS_G12A |
| 12Arg (GGT > CGT) | KRAS_G12R |
| 13Asp (GGC > GAC) | KRAS_G13D |

A person skilled in the art is easily in the position to identify these and further KRAS-mutations denominated in a format as in the above Kit and to correlate these mutations to the activating K-RAS mutations described herein.

An exemplary detection of KRAS mutations may be performed as follows: serial sections are cut from the paraffin block and DNA is extracted. The DNA is then amplified using a combination of Scorpions™ and ARMS™ (allele specific PCR) technologies to detect the 7 most common nucleotide substitutions, all present in codons 12 and 13 of the KRAS gene. Such a kind of assay is preferably sensitive to 1% of mutant DNA in a background of wild type genomic DNA.

A positive result in the detection method described above indicates the presence of an activating mutation in the KRAS gene (see also Mitsudomi T and Yatabe Y (2007) Cancer Sci 98:1817-24; Massarelli E et al (2007) Clin Cancer Res 13:2890-96; Finocchiaro G et al (2007) J Clin Oncol, ASCO Annual Meeting Proceedings 25 (18S):4021).

Further kits to be used for detecting KRAS mutations, are commercially available, for example from DxS Ltd, 48 Grafton Street, Manchester, UK. Such kits may be used for detecting mutations in oncogenes. In particular, TheraScreen may be used for detecting mutations in the EGFR and K-RAS genes. Further validated biomarker kits are commercially available for EGFR, RAS, RAF, BCR-ABL and other genes.

As described herein below and shown in the appended example, these methods for determining the susceptibility to (an) HSP90 inhibitor(s)/responsiveness to treatment with (an) HSP90 inhibitor(s) may comprise in a first step contacting cell(s), tissue(s) or cell culture(s) with an HSP90 inhibitor or exposing cell(s), tissue(s) or cell culture(s) to an HSP90 inhibitor A person skilled in the art knows how the contacting with/exposing to an HSP90 inhibitor is to be performed. For example, the cell(s), tissue(s) or cell culture(s) may be incubated with an HSP90 inhibitor comprised in a composition with appropriate diluents, stabilizers and/or carriers. The molar concentration of the HSP90 inhibitor to be used in the contacting/exposing step (comprising, for example, incubating the cell(s), tissue(s) or cell culture(s) with an HSP90 inhibitor) are, of course, dependent on the nature of the HSP90 inhibitor. Also diluents, stabilizers and/or carriers to be optionally added in the contacting/exposing step will depend on the nature of the HSP90 inhibitor. However, a person skilled in the art will know which diluents, stabilizers and/or carriers and the like are to be added and will also be aware of appropriate concentrations of diluents, stabilizers and/or carriers and also of the HSP90 inhibitor(s) to be used in the selection method/method for determining the responsiveness of the present invention. Other methods for exposing cells to the HSP90 inhibitor may be the use of cell-matrix or compound arrays, where cells are matrix-bound in array format and can therefore be simultaneously exposed to multiple inhibitors or multiple concentrations of inhibitors or where compounds are matrix-bound in array format an can therefore be simultaneously exposed to multiple types of cells or aliquots of cells.

Also the use of high throughput screening (HTS) is envisaged in context of the present invention, in particular the screening methods of cell(s), tissue(s) and/or cell culture(s) for responsiveness/sensitivity to an HSP90 inhibitor. Suitable (HTS) approaches are known in the art. An exemplary protocol for such a screening method is also provided in the appended examples; a person skilled in the art is readily in the position to adapt this protocol or known HTS approaches to the performance of the methods of the present invention.

Screening-assays are usually performed in liquid phase, wherein for each cell/tissue/cell culture to be tested at least one reaction batch is made. Typical containers to be used are micro titer plates having for example, 384, 1536, or 3456 wells (i.e. multiples of the “original” 96 reaction vessels).

Robotics, data processing and control software, and sensitive detectors, are further commonly used components of a HTS device. Often robot system are used to transport micro titer plates from station to station for addition and mixing of sample(s) and reagent(s), incubating the reagents and final readout (detection). Usually, HTS can be used in the simultaneous preparation, incubation and analysis of many plates.

The assay can be performed in a singly reaction (which is usually preferred), may, however, also comprise washing and/or transfer steps. Detection can be performed taking advantage of radioactivity, luminescence or fluorescence, like fluorescence-resonance-energytransfer (FRET) and fluorescence polarisation (FP) and the like. The biological samples described herein can also be used in such a context. In particular cellular assays and in vivo assays can be employed in HTS. Cellular assays may also comprise cellular extracts, i.e. extracts from cells, tissues and the like. However, preferred herein is the use of cell(s) or tissue(s) as biological sample (in particular a sample obtained from a patient/subject suffering or being prone to suffer from cancer), whereas in vivo assays (wherein suitable animal models are employed, e.g. the herein described mouse models) are particularly useful in the validation/monitoring of the treatment with an HSP90 inhibitor. Depending on the results of a first assay, follow up assays can be performed by re-running the experiment to collect further data on a narrowed set (e.g. samples found “positive” in the first assay), confirming and refining observations.

HTS cannot only be employed in identifying cell(s), tissue(s) and/or animal(s) susceptible/responsive to HSP90 inhibitors, or in monitoring the efficacy of a treatment of cancer as described herein; FITS is also useful in identifying further HSP90 inhibitors to be used herein. The screening of compound libraries with usually several hundred thousands of substances takes usually between days and weeks. An experimental high throughput screen may be supplemented (or even be replaced) by a virtual screen. For example, if the structure of the target molecule (e.g. HSP90) is known methods can be employed, which are known under the term “docking”. If the structure of several target-binding molecules is known (e.g. the herein described HSP90 inhibitors) methods for Pharmacophor-Modelling can be used aiming at the development new substances which also bind to the target molecule. A suitable readout in animal (in vivo) models is tumor growth (or respectively the complete or partial inhibition of tumor growth and/or its remission).

High-throughput methods for the detection of mutations (e.g. KRAS) involve massively parallel sequencing approaches, such as the “picotiter plate pyrosequencing”. This approach relies on emulsion PCR-based clonal amplification of a DNA library adapted onto micron-sized beads and subsequent pyrosequencing-by-synthesis (Thomas R K et al. Nature Med 2007) of each clonally amplified template in a picotiter plate, generating over 200,000 unique clonal sequencing reads per experiment. Furthermore, mass spectrometric genotyping approaches (Thomas R K et al.; Nat Gen 2007) and other next generation sequencing methods (Marguerat S et al.; Biochem Soc Trans 2008).

The meaning of the terms “cell(s)”, “tissue(s)” and “cell culture(s)” is well known in the art and may, for example, be deduced from “The Cell” (Garland Publishing, Inc., third edition). Generally, the term “cell(s) used herein refers to a single cell or a plurality of cells. The term “plurality of cells” means in the context of the present invention a group of cells comprising more than a single cell. Thereby, the cells out of said group of cells may have a similar function. Said cells may be connected cells and/or separate cells. The term “tissue” in the context of the present invention particularly means a group of cells that perform a similar function. The term “cell culture(s)” means in context of the present invention cells as defined herein above which are grown/cultured under controlled conditions. Cell culture(s) comprise in particular cells (derived/obtained) from multicellular eukaryotes, preferably animals as defined elsewhere herein. It is to be understood that the term “cell culture(s)” as used herein refers also “tissue culture (s)” and/or “organ culture(s)” an “organ” being a group of tissues which perform the some function.

Preferably, the cell(s), tissue(s) or cell culture(s) to be contacted with/exposed to an HSP90 inhibitor comprise/are derived from or are (a) tumor cell(s). The tumor cells may, for example, be obtained from a biopsy, in particular a biopsy/biopsies from a patient/subject suffering from non-small cell lung cancer, lung adenocarcinoma, pancreatic cancer, colorectal cancer, breast cancer, leukemias, prostate cancer, lymphomas, brain tumors, pediatric tumors or sarcomas or, though less preferred a patient/subject being prone to suffer from said disorders. It is preferred herein that said subject is a human. The term “mammalian tumor cell(s)” used herein refers to (a) tumor cell(s) which is derived from or is a tumor cell from a mammal, the term mammal being derived herein below. As described herein above in respect of “cell(s)”, “tissue(s)” and “cell culture(s)” the “mammalian tumor cells” may be obtained from a biopsy, in particular a biopsy/biopsies from a patient/subject suffering from non-small cell lung cancer, lung adenocarcinoma, pancreatic cancer, colorectal cancer, breast cancer, leukemias, prostate cancer, lymphomas, brain tumors, pediatric tumors or sarcomas or, though less preferred a patient/subject being prone to suffer from said disorders. The term “tumor cell” also relates to “cancer cells”.

Generally, said tumor cell or cancer cell may be obtained from any biological source/organism, particularly any biological source/organism, suffering from the above-mentioned non-small cell lung cancer, lung adenocarcinoma, pancreatic cancer, colorectal cancer, breast cancer, leukemias, prostate cancer, lymphomas, brain tumors, pediatric tumors or sarcomas.

Preferably, the (tumor) cell(s) or (cancer) cell to be contacted is (are) obtained/derived from an animal. More preferably, said (tumor)/(cancer) cell(s) is (are) derived from a mammal. The meaning of the terms “animal” or “mammal” is well known in the art and can, for example, be deduced from Wehner und Gehring (1995; Thieme Verlag). Non-limiting examples for mammals are even-toed ungulates such as sheep, cattle and pig, odd-toed angulates such as horses as well as carnivors such as cats and dogs. In the context of this invention, it is particularly envisaged that DNA samples are derived from organisms that are economically, agronomically or scientifically important. Scientifically or experimentally important organisms include, but are not limited to, mice, rats, rabbits, guinea pigs and pigs.

The tumor cell(s) may also be obtained from primates which comprise lemurs, monkeys and apes. The meaning of the terms “primate”, “lemur”, “monkey” and “ape” is known and may, for example, be deduced by an artisan from Wehner und Gehring (1995, Thieme Verlag). As mentioned above, the tumor or cancer cell(s) is (are) most preferably derived from a human being suffering from the above-mentioned non-small cell lung cancer, lung adenocarcinoma, pancreatic cancer, colorectal cancer, breast cancer, leukemias. In context of this invention particular useful cells, in particular tumor or cancer cells, are, accordingly, human cells. These cells can be obtained from e.g. biopsies or from biological samples but the term “cell” also relates to in vitro cultured cells.

Preferred cell(s) or cell culture(s) also used in the appended example are shown in appended FIG. 18. Said cell(s)/cell line(s) include but are not limited to H2030, H358, SKLU1, HCC44, H1944. H157, H1355, H2122, H441, HCC461, A427, H1734, H1792, H460, DV-90, Calu1, H2009, A549, LCLC97TM1, HCC515, HOP62, Calu6, H647, HCC1171, H2444 and H2887. These cell lines are well known in the art and may be obtained from ATCC and/or DSMZ and/or from the U.S. National Cancer Institute as part of the NCI60 collection of cancer cell lines (www.lgcpromochem-atcc.com/; www.dsmz.de/; dtp.nci.nih.gov/docs/misc/common_files/cell_list.html).

These selection methods or method for determining the responsiveness to treatment with an HSP90 inhibitor may also comprise determining the activity/level of expression of HSP90, wherein the activity/level of expression of HSP90 is indicative whether the cell(s), tissue(s) or cell culture(s) is (are) susceptible to an HSP90 inhibitor or is responsive to treatment with an HSP90 inhibitor. The term “activity of HSP90” used herein refers to the activity of a HSP90 protein (protein encoded by a hsp90 gene). The term “expression of HSP90” is used herein interchangeably with “expression of HSP90 gene” and refers to the expression of the hsp90 gene. The definition given herein above in respect of “activity of HSP90”/“expression of HSP90” applies to “activity of KRAS”/“expression of KRAS”, “activity of EGFR”/“expression of EGFR” and “activity of BRAF”/“expression of BRAF”, mutatis mutandis. The It is to be understood that the activity/expression level of HSP90 determined in (a) cell(s), (a) tissue(s) or (a) cell culture(s) contacted with/exposed to an HSP90 inhibitor is compared with the activity/expression level of HSP90 in (a) control cell(s), (a) tissue(s) or (a) cell culture(s), i.e. cell(s), (a) tissue(s) or (a) cell culture(s) not contacted with/exposed to an HSP90 inhibitor. A skilled person will be aware of means and methods for performing such tests and selecting appropriate controls. Preferably, the control cell(s), (a) tissue(s) or (a) cell culture(s) will be identical to the cell(s), (a) tissue(s) or (a) cell culture(s) to be tested as described herein with the only exception that the control (s), (a) tissue(s) or (a) cell culture(s) are not contacted with/exposed to the HSP90 inhibitor.

Preferably, decreased HSP90 activity/expression levels of HSP90 proteins/polypeptides and/or HSP90 polynucleotides/nucleic acid molecules are indicative of susceptibility to an HSP90 inhibitor or of responsiveness to treatment with an HSP90 inhibitor. It is preferred herein that the HSP90 activity/expression level is decreased by at least 10%, 20%, 30%, 40%, 50%, 60%, 70%, 80% and most preferably by at least 90% in cell(s), (a) tissue(s) or (a) cell culture(s) contacted with/exposed to an HSP90 inhibitor compared with the activity/expression level of HSP90 in (a) control cell(s), (a) control tissue(s) or (a) control cell culture(s). It is of note that the HSP90 activity must not necessarily correlate with the expression level. Thus, it may be, that HSP90 activity is decreased in the presence of an HSP90 inhibitor even though HSP90 expression is increased. However, a person skilled of the art will be aware of this and preferably evaluate HSP90 activity (i.e. activity/function of the HSP90 protein) when determining susceptibility to an HSP90 inhibitor or of responsiveness to treatment with an HSP90 inhibitor.

As mentioned, a person skilled in the art will be aware of corresponding means and methods for detecting and evaluating the HSP90 activity/expression level. Exemplary methods to be used include but are not limited to molecular assessments such as Western Blots, Northern Blots, Real-Time PCR and the like.

If the gene product is an RNA, in particular an mRNA (e.g. unspliced, partially spliced or spliced mRNA), determination can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR. These and other suitable methods for binding (specific) mRNA are well known in the art and are, for example, described in Sambrook and Russell (2001, loc. cit.). A skilled person is capable of determining the amount of the component, in particular said gene products, by taking advantage of a correlation, preferably a linear correlation, between the intensity of a detection signal and the amount of the gene product to be determined.

In case the component is a polypeptide/protein, quantification can be performed by taking advantage of the techniques referred to above, in particular Western blotting techniques. Generally, the skilled person is aware of methods for the quantitation of (a) polypeptide(s)/protein(s). Amounts of purified polypeptide in solution can be determined by physical methods, e.g. photometry. Methods of quantifying a particular polypeptide in a mixture rely on specific binding, e.g of antibodies. Specific detection and quantitation methods exploiting the specificity of antibodies comprise for example immunohistochemistry (in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction. Alternatively, protein quantitation methods may involve but are not limited to mass spectrometry or enzyme-linked immunosorbant assay methods.

In one embodiment, the present invention relates to an in vitro method for the identification of a responder for or a patient sensitive to an HSP90 inhibitor, said method comprising the following steps: (a) obtaining a sample from a patient suspected to suffer from or being prone to suffer from a cancer characterized by the presence of at least one mutation in the KRAS gene, (in particular an activating mutation) and, optionally, in the EGFR and/or the BRAF gene (again in particular an activating mutation in the KRAS gene); and (b) evaluating the presence of at least one mutation in the KRAS gene, and, optionally, the EGFR and/or the BRAF gene; whereby said mutation in the KRAS gene alone or in addition to a mutation in the EGFR and/or the BRAF gene is indicative for a responding patient or is indicative for a sensitivity of said patient to an HSP90 inhibitor. As pointed out herein, in particular activating mutations in the KRAS gene are of importance in the embodiments provided herein.

Said sample may, for example, be obtained by (a) biopsy (biopsies). Preferably, said sample is obtained from a patient suspected to suffer from or being prone to suffer from cancer. In particular said cancer may be suspected of being characterized by the presence of at least one mutation in the KRAS gene. The cancer may, in addition to the presence of at least one mutation in the KRAS gene be characterized by the presence of at least one mutation in the EGFR gene and/or the BRAF gene. It is preferred herein that said sample is obtained from (a) tumor(s) and, accordingly, is (a) tumor cell(s) or (a) tumor tissue(s). Preferred tumors the sample may be obtained from are non-small cell lung cancer, lung adenocarcinoma, pancreatic cancer, colorectal cancer, breast cancer, leukemias, prostate cancer, lymphomas, brain tumors, pediatric tumors or sarcomas which have already been described above are. A person skilled in the art is easily in the position to identify cancers characterized by the presence of at least one mutation in the KRAS gene using standard techniques known in the art and methods described herein.

Another embodiment of the present invention relates to the use of an oligo- or polynucleotide capable of detecting (a) mutation(s) of at least one mutation in the KRAS gene for diagnosing sensitivity to an HSP90 inhibitor as defined herein above. Preferably, the oligonucleotide(s) is (are) about 15 to 100 nucleotides in length. A person skilled in the art is, based on his general knowledge and the teaching provided herein, easily in the position to identify and/or prepare (a) an oligo- or polynucleotide capable of detecting at least one mutation in the KRAS gene and, optionally, in the EGFR and/or BRAF gene and/or further genes. In particular these oligo- or polynucleotides may be used as probe(s) in the detection methods described herein A skilled person will know, for example, computer programs which may be useful for the identification of corresponding probes to be used herein. For example, the KRAS nucleic acid sequence (SEQ ID NO: 1 or 157) may be used in this context for identifying specific probes for detecting mutations in the KRAS gene. Likewise probes may be identified using the corresponding sequences coding for EGFR (SEQ ID NO: 149, 151, 153, 155) and BRAF (SEQ ID NO:147), respectively. Exemplary KRAS, EGFR and BRAF nucleic acid sequences are available on corresponding databases, such as the NCBI database (www.ncbi.nlm.nih.gov/sites/entrez). Accordingly, a KRAS nucleic acid sequence can be found in this database under the number “Gene ID: 3845”. Corresponding EGFR and BRAF sequences can be found under the number “Gene ID 1956” and “Gene ID: 673”, respectively. As mentioned, exemplary sequences of a KRAS nucleic acid sequence, EGFR nucleic acid sequence and BRAF nucleic acid sequence are also depicted in SEQ ID NOs: 1 and 157, SEQ ID NO: 149, 151, 153, 155 and SEQ ID NO:147, respectively.

In a particular embodiment, a method of monitoring the efficacy of a treatment of a cancer is disclosed herein, whereby said cancer is characterized by the presence of at least one mutation in the KRAS gene, and, optionally, in the EGFR and/or the BRAF gene in a subject/patient suffering from said disorder or being prone to suffering from said disorder comprising the steps of
a) determining in a cell or tissue sample obtained from said subject/patient the activity/expression level of KRAS, and, optionally of EGFR and/or BRAF; and
b) comparing the activity/expression level of said at least one marker gene determined in a) with a reference or control activity/expression level of KRAS, and, optionally of EGFR and/or BRAF, wherein the extent of the difference between said activity/expression level determined in a) and said reference activity/expression level is indicative for said efficacy of a treatment of said cancer.

The term “activity” as used herein refers to the activity of a protein as described elsewhere herein, whereas the term expression level refers to expression on a protein level (e.g. to be determined by Western Blots and the like) or transcriptional level (e.g. spliced, unspliced or partially spliced mRNA, which may be determined by Northern Blots, Real time PCR and the like). Marker genes referred to in the context of the monitoring methods or methods of predicting the efficacy of a treatment are in particular the KRAS gene, and, optionally the EGFR gene and/or BRAF gene. Again, in particular activating mutations in the KRAS gene are of importance in the embodiments provided herein.

The method of monitoring the efficacy of a treatment of a cancer may comprise a step of determining in a cell or tissue sample obtained from a subject/patient suffering from said cancer (e.g. a biopsy) the presence of at least one mutation in the KRAS gene. The monitoring method may comprise, in addition to determining at least one mutation in the KRAS gene, determining the presence of at least one mutation in the EGFR gene and/or BRAF gene. For example, a mutation in the KRAS gene (and, optionally, in the EGFR gene and/or BRAF gene) may be present in a sample before start of the treatment of a cancer. During or after treatment of the cancer, the tumor cells having the mutation(s) in the KRAS gene (and, optionally, in the EGFR gene and/or BRAF gene) are erased or otherwise depleted. Thus, the absence of a detectable mutation in the KRAS gene (and, optionally, in the EGFR gene and/or BRAF gene) in a sample (cell samples/biopsy samples and the like) obtained from a subject/patient during or after treatment of a cancer is indicative of the efficacy of the treatment.

The present invention also relates to a method of predicting the efficacy of a treatment of a cancer characterized by the presence of at least one mutation in the KRAS gene, and, optionally, in the EGFR and/or the BRAF gene for a subject/patient suffering from said disorder or being prone to suffering from said disorder comprising the steps of a) determining in a cell or tissue sample obtained from said subject/patient the activity of KRAS, and, optionally, EGFR and/or BRAF activity/expression level of at least one marker gene selected from the group consisting of KRAS, EGFR and/or BRAF; and b) comparing the activity of KRAS, and, optionally, EGFR and/or BRAF activity/expression level of said at least one marker gene determined in a) with a reference activity or reference expression level of KRAS, and, optionally, EGFR and/or BRAF reference activity of reference expression level, optionally determined in a cell or tissue sample obtained from a control subject/patient (responder and/or non-responder), wherein the extent of the difference between said activity or expression level determined in a) and said reference or control activity or reference or control expression level is indicative for the predicted efficacy of a treatment of cancer. Also here, it is to be pointed out that in particular activating KRAS gene mutations are of interest in this embodiment.

The treatment of cancer characterized by the presence of at least one mutation in the KRAS gene, and, optionally, in the EGFR and/or the BRAF gene may comprise the administration of an HSP90 inhibitor as defined herein above.

it has been demonstrated in context of this invention that mutations in the KRAS gene as disclosed herein act as markers/predictors for susceptibility to an HSP90 inhibitor. In particular a responder for or a patient sensitive to an HSP90 inhibitor may be identified in accordance with the present method. Accordingly, the present invention provides the possibility to recognize (aberrant) changes of KRAS activity immediately once they occur, for example, by determining the activity of said marker gene(s). In an alternative, (aberrant) changes of HSP90 activity may be recognized.

The activity of (mutated) KRAS and, optionally, (mutated) EGFR, (mutated) BRAF and/or HSP90 may, not only be determined by measuring the expression level but also, be determined, for example, by measuring substrate turnover in case of HSP90 or EGFR or by measuring GTPase activity of KRAS or by measuring the activation of downstream signaling pathway members, e.g., by determining the level of phospho-Akt or phospho-Erk. in case of KRAS or the phosphorylation levels of BRAF or Erk in the case of BRAF. Means and methods for determining the activity of said proteins are well known in the art and may, for example, be deduced from Lottspeich (Spektrum Akademischer Verlag, 1998). As mentioned herein above, determining the activity may comprise determining the expression level. Accordingly, in an alternative, the expression status (i.e. expression of gene products such as mRNA and/or proteins) of (mutated) KRAS, (mutated) EGFR, (mutated) BRAF and/or HSP90 can be determined by standard techniques, (A) mutation(s) in the KRAS gene and, optionally, EGFR gene and/or BRAF gene does not necessarily correlate with a change in the expression level of these genes. In context of the present invention it is therefore preferred that the activity of (mutated) KRAS and, optionally, (mutated) EGFR, (mutated) BRAF is not determined by measuring the expression status of these genes but by alternative methods which reflect, for example, the enzymatic activity of the corresponding proteins. It is of note that, for example, the enzymatic activity of proteins encoded by a mutated KRAS gene, and optionally a mutated EGFR gene and/or mutated BRAF gene may differ from that of the respective proteins encoded by wild type genes (i.e. reference activity) without a change in the expression level. Preferably, mutated KRAS and, optionally, mutated EGFR, mutated BRAF show an increased activity compared to wild type KRAS and, optionally, wild type EGFR and/or wild type BRAF (controls). In particular the enzymatic activity and/or the expression level described herein above are increased. Exemplary ranges of changes in the activity compared to “normal” activity (i.e. activity of wild-type KRAS, and optionally, wild-type EGFR and/or wild-type BRAF) are described herein below. The term “Wild type” used herein refers to the “original” nucleic acid sequences and amino acid sequences/proteins encoded thereby, in particular nucleic acid sequences without the mutations described herein in context of “KRAS mutations” “EGFR mutations” and “BRAF mutations”. An “increased activity” according to the present invention relates to a higher activity, in particular activating mutation in the KRAS, than shown by the identified wild-type (KRAS) gene.

In addition or in alternative to the activity of the herein above described proteins encoded by the “marker genes” KRAS, EGFR and/or BRAF also the activity/expression level of c-RAF and/or Aktin may be measured/determined. C-RAF genes and Aktin genes and corresponding proteins are well known in the art and the expression status of these proteins has also been determined in the appended example. C-RAF and/or Aktin may, accordingly, also be used as “marker genes” in context of the present invention. All explanations given herein below in respect of KRAS, EGFR and/or BRAF in context of methods of monitoring the efficacy of a treatment or methods of predicting the efficacy of a treatment also apply to C-RAF and/or Aktin, mutatis mutandis.

Based on these findings, the present invention provides the particular advantages that, by determining the expression level or activity of KRAS, and, optionally, EGFR and/or BRAF activity or expression level of at least one of said marker genes in accordance with this invention, (aberrant) changes of mutated KRAS and, optionally, mutated EGFR, mutated BRAF and/or HSP90 activity/expression level can be recognized early, i.e. that the efficacy of a treatment of a cancer characterized by the presence of at least one mutation in the KRAS and, optionally, in the EGFR gene, and/or BRAF gene can be monitored early and that the efficacy of a treatment of said cancer can be predicted early. Hence, also a possible resistance to the treatment can be recognized early by using the means and method of this invention.

In a particular embodiment, the present invention relates to corresponding means, methods and uses which are based on the early recognition of (aberrant) changes of mutated KRAS and, optionally, mutated EGFR, mutated BRAF and/or HSP90 activity/expression level of the respective genes. The possibility of recognizing (aberrant) changes of mutated KRAS and, optionally, mutated EGFR, mutated BRAF and/or HSP90 activity/expression level early, provides several advantages, like a higher lifespan/likelihood of survival of the subject/patient (for example due to the notice of possible treatment failures and a corresponding change of the treatment regimen) and the possibility of a more efficient therapy (for example due to the possibility to avoid/recognize treatment failures early and, hence, to correspondingly change the treatment regimen early in therapy, i.e. to timely switch to a more suited inhibitor, to discontinue an expensive, ineffective treatment early after diagnosis and to opt for alternative therapy).

In context of the above embodiments of this invention, “early” particularly means prior to (the onset of) a (complete or partial) cytogenetic or haematological response or a response measured by any type of imaging technique and/or prior to the outbreak of the cancer characterized by the presence of at least one mutation in the KRAS and, optionally, in the EGFR gene, and/or BRAF gene (or susceptibility thereto).

For example, “early” monitoring the efficacy of a therapy/treatment of said cancer may be at least 1, at least 2, at least 3, at least 4, at least 5, at least 6, at least 7, at least 10, or at least 14 days prior to (the onset of) a (partial) cytogenetic or haematological response or a response measured by any type of imaging technique to said therapy/treatment and/or at least 1, at least 2, at least 3, at least 4, at least 5, at least 6, at least 7, at least 10, at least 12, at least 15, or at least 18 month prior a complete cytogenetic or haematological response or a response measured by any type of imaging technique to said therapy/treatment (of the patient or control patient (responder)), wherein the longer periods are preferred.

Alternatively, “early” monitoring the efficacy of a therapy/treatment of said cancer may also be at most 1, at most 2, at most 3, at most 4, at most 5, at most 6, at most 7, at most 10, or at most 14 days after (onset of) the therapy/treatment of said cancer, wherein the shorter periods are preferred. Most preferably, it is envisaged to already monitor the efficacy of a therapy/treatment of said cancer at the day the therapy/treatment was initiated, i.e. once the mutated KRAS and, optionally, mutated EGFR, mutated BRAF and/or HSP90 activity/expression level changes upon said therapy/treatment.

In the following, a non-limiting example of a scheme for (early) monitoring the efficacy of a therapy/treatment of the cancer defined herein in accordance with this invention is provided:
When monitoring the therapy/treatment of said cancer (for example therapy/treatment based on an HSP90 inhibitor as described herein (e.g. 17-AAG)), activity of KRAS, and, optionally, EGFR and/or BRAF activity/expression level(s) of the marker gene(s) (i.e. mutated KRAS and, optionally, mutated EGFR, mutated BRAF and/or HSP90) may be determined daily during the first week after initiation of the therapy/treatment, weekly during the first month of the therapy/treatment and, afterwards, monthly.
The reference activity/expression level may be taken at the day the therapy/treatment is initiated, from the subject/patient to be treated and/or from a corresponding control subject/patient (responder/non-responder); see below.
If a rise in marker levels is observed in two consecutive samples, or if decrease in marker level is not fast enough (for example not as fast as that of responder or not sufficiently faster than that of a non-responder), change of treatment regimen may be considered.

It is of note that this example is in no way limiting. The skilled person is readily in the position to adapt this scheme to the particular requirements relevant for each individual case, based on the teaching provided herein an on his common general knowledge.

For example, “early” predicting the efficacy of a therapy/treatment of the cancer defined herein may be at least 1, at least 2, at least 3, at least 4, at least 5, at least 6, at least 7, at least 10, or at least 14 days prior to (the onset of) a (partial) cytogenetic or haematological response to said therapy/treatment and/or at least 1, at least 2, at least 3, at least 4, at least 5, at least 6, at least 7, at least 10, at least 12, at least 15, or at least 18 month prior a complete cytogenetic or haematological response or a response measured by any type of imaging technique to said therapy/treatment, wherein the longer periods are preferred.

Alternatively, “early” predicting the efficacy of a therapy/treatment of the cancer defined herein may also be at most 1, at most 2, at most 3, at most 4, at most 5, at most 6, at most 7, at most 10, or at most 14 days after (onset of) the therapy/treatment of the cancer defined herein, wherein the shorter periods are preferred. Most preferably, it is envisaged to already monitor the efficacy of a therapy/treatment of said cancer at the day the therapy/treatment was initiated, i.e. once the mutated KRAS and, optionally, mutated EGFR, mutated BRAF and/or HSP90 activity/expression level changes upon said therapy/treatment.

Furthermore, “early” predicting the efficacy of a therapy/treatment of the cancer defined herein may also be at most 1, at most 2, at most 3, at most 4, at most 5, at most 6, at most 7, at most 10, or at most 14 days after diagnosis of the cancer, wherein the shorter periods are preferred. Most preferably, it is envisaged to already predict the efficacy of a therapy/treatment of said cancer at the day of diagnosis.

As mentioned, the present invention is particularly useful for monitoring the efficacy of a therapy/treatment of the cancer as defined herein. Corresponding means, uses and methods are provided herein. In general, monitoring the efficacy of a certain kind of therapy/treatment is regularly applied in clinical routine. Hence, the skilled person is aware of the meaning of monitoring the efficacy of a certain kind of therapy/treatment. In context of this invention, the meaning of the term “monitoring” encompasses the meaning of terms like “tracking”, “discovering” etc. In particular, the term “monitoring the efficacy of a therapy/treatment of a cancer characterized by the presence of at least one mutation in the KRAS gene, and, optionally, in the EGFR and/or the BRAF gene” as used herein refers to monitoring whether a subject/patient suffering from said disorder (or being prone to suffering from said cancer) responds at all to a therapy/treatment of said disorder and/or how the course of said respond is (e.g. how fast/slow the respond is and/or to what extent the respond is).

The present invention is further useful for predicting the efficacy of a therapy/treatment of the cancer as defined herein. Corresponding means, uses and methods are also provided herein. In general, predicting the efficacy of a certain kind of therapy/treatment is highly desired in clinical routine, since it allows for preventing the disorder and/or increasing the efficiency of a therapy/treatment and hence, leads to savings in cost and time and to a higher lifespan/likelihood of survival/‘Genesung’ of the affected patient. The definitions given with respect to the term “efficacy of a therapy/treatment of a cancer characterized by the presence of at least one mutation in the KRAS gene, and, optionally, in the EGFR and/or the BRAF gene” provided herein apply here, mutatis mutandis. In context of this invention, the term “predicting the efficacy of a therapy/treatment of a cancer characterized by the presence of at least one mutation in the KRAS gene, and, optionally, in the EGFR and/or the BRAF gene for a subject/patient” is used in basically the same sense like determining whether, and/or to what extent, a subject/patient exhibits susceptibility to such therapy/treatment, i.e. whether said subject/patient will or would respond at all to a therapy/treatment of said disorder and/or how the course of said respond will or would be (e.g. how fast/slow the respond is and/or to what extent the respond is). In particular, a subject/patient exhibits susceptibility to said cancer in accordance with this invention, when its mutated KRAS and, optionally, mutated EGFR, mutated BRAF and/or HSP90 activity/expression level is aberrant.

In one embodiment, the “predicting the efficacy of a therapy/treatment of the cancer defined herein” in accordance with this invention may be performed after initiation of the therapy/treatment, i.e. during the already ongoing therapy/treatment. In particular, said “predicting” may be performed during the herein described monitoring the efficacy of a therapy/treatment of said cancer, preferably early after the beginning of said monitoring. Thereby, the predicting may be based on results from said monitoring obtained at a certain point in time of the ongoing therapy/treatment. Preferably, said point in time is an early point in time, like, for example that point in time, when a first result from said monitoring has been obtained. In cases where the “predicting the efficacy of a therapy/treatment of the cancer defined herein” is performed during an already ongoing therapy/treatment, it refers to the following/subsequent efficacy of said therapy/treatment.

In another embodiment, the “predicting the efficacy of a therapy/treatment of the cancer defined herein” in accordance with this invention may be performed (immediately) after diagnosis but, however, prior to initiation of the therapy/treatment. In such cases, “predicting the efficacy of a therapy/treatment of said cancer” refers to the efficacy of a therapy/treatment which has not yet been initiated (or has been initiated substantially at the same point in time when the “predicting” was performed.

In context of this embodiment of the invention, one non-limiting example of a healthy control subject/patient is one having (a) wild-type KRAS gene(s), in particular (a) wild-type KRAS gene(s) with respect to a mutation leading to an aberrant activity/aberrant expression of KRAS. In other words said healthy control subject/patient is one not having a form of a KRAS gene with a mutation leading to an aberrant activity/aberrant expression of KRAS (for example the presence of the KRAS_G12C, KRAS_G12R, KRAS_G12D, KRAS_G1.2A, KRAS_G12S, KRAS_G12V, KRAS_G13D, KRAS_G13S, KRAS_G13C, KRAS_G13V, KRAS_Q61H, KRAS_Q61R, KRAS_Q61P, KRAS_Q61L, KRAS_Q61K, KRAS_Q61E, KRAS_A59T and KRAS_G12F mutations). These mutations are also shown in appended SEQ ID NOs 3 to 78.

In accordance with the above, the reference activity or expression level of KRAS, and, optionally (i.e. in addition to the determination of the activity/expression level of KRAS) EGFR and/or BRAF reference activity or reference expression level with respect to the means, methods and uses of monitoring the efficacy of a treatment of a cancer as defined herein, is that determined in (a sample of) the corresponding healthy control subject, i.e. is the “normal” activity/expression level.

It is to be understood that an aberrant activity or expression level of the marker genes described herein means that the activity or expression level of KRAS, and, optionally (i.e. in addition to the determination of the activity/expression level of KRAS) EGFR and/or BRAF activity/expression level as described herein is different from the above described reference activity or reference expression level of KRAS, and, optionally, EGFR activity/expression level and/or BRAF activity/expression level. The reference activity or reference expression level of these marker genes is in this context, accordingly, the “normal” activity/expression level. In particular with respect to the herein disclosed means, methods and uses of monitoring/predicting the efficacy of a treatment of the cancer as defined herein, the control subject/patient is, in one embodiment, envisaged to be a subject/patient suffering from said cancer or being prone to suffering from said cancer, i.e. a subject/patient having, for example, an aberrant activity/expression level of KRAS and, hence, not a “normal” activity of KRAS activity or normal KRAS expression level as described in accordance with this invention.

Thereby, it is clear that “different” activity or expression level means higher or lower, depending on whether the cancer defined and described herein comes along with an up- or down-regulated activity of KRAS, and optionally (i.e. in addition to the determination of the activity/expression level of KRAS), EGFR and/or BRAF activity or expression level.

In this context, “different”, “higher” or “lower” means different, higher or lower than the normal (range of) activity or expression of KRAS, and, optionally, (i.e. in addition to the determination of the activity/expression level of KRAS) EGFR and/or BRAF activity or expression level. For example, different, higher or lower means at least 1.5 fold, at least 2 fold, at least 2.5 fold, at least 3 fold, at least 4 fold, at least 5 fold, at least 7 fold, at least 10 fold, at least 15 fold, at least 25 fold, at least 50 fold, at least 100 fold, at least 200 fold different, higher or lower, wherein the higher values are preferred.

Whether the activity or expression level changes (i.e., in a certain direction like “higher” or “lower”) and to which extent the activity or expression level of KRAS changes can easily be deduced by the skilled person based on the teaching provided herein and the common general knowledge. As pointed out herein, in addition to the KRAS modifications also the expression level and/or activity of EGFR and/or BRAF may be measured assessed. This optional measurement or assessment of EGFR and/or BRAF activity and/or expression level, can also be easily deduced by the skilled person based on the teaching provided herein and the common general knowledge.

It is particularly preferred in this context that the control subject/patient is subjected to the same treatment of the cancer described and defined herein as the subject/patient itself and/or that it is known whether the control subject/patient is a responder or non-responder to this treatment.

Whether a subject/patient is a “responder” or “non-responder” with respect to a certain kind of cancer treatment/therapy can be evaluated by the skilled person on the basis of his common general knowledge and/or the teaching provided herein. Accordingly, the patient responds to cancer treatment/therapy, if expression/activity of KRAS, and optionally. EGFR and/or BRAF is reduced upon said treatment/therapy. Preferably, the expression/activity of KRAS, and optionally, EGFR and/or BRAF is reduced to control expression/activity (e.g. determined in a sample obtained from a person not suffering from said cancer). In other words, a reduction in expression level or activity of KRAS, and optionally, EGFR and/or BRAF is indicative for a successful treatment/therapy. A skilled person is readily in the position to determine whether a patient responds to cancer treatment/therapy by evaluation of the expression level or activity of KRAS, and optionally, of EGFR and/or of BRAF. In addition to the evaluation of said expression level or activity, a person skilled in the art may also determine cytological/haematological parameters characteristic for a specific cancer in order to assess whether a patient responds to cancer treatment/therapy.

In contrast, a patient who does not respond to treatment/therapy does not show a reduced expression level or activity of KRAS, and optionally, a reduced expression level or reduced activity of EGFR and/or of BRAF upon said treatment/therapy. This is in contrast to “responders/responding patients”, showing such a reduced expression level and/or such a reduced activity.

In particular, a “responder” may be a subject/patient whose cytological/haematological parameters and/or (aberrant) KRAS activity or expression level (and hence the corresponding marker gene expression level(s)) activity of KRAS, and, optionally, EGFR and/or BRAF change towards the their “normal” activity/(protein or mRNA expression) level(s) (in a sufficient manner) upon the cancer treatment/therapy. In one specific embodiment, a “responder” may be a subject/patient not suffering from one of the herein defined resistances. In particular, a “non-responder” may be a subject/patient whose cytological/haematological parameters and/or (aberrant) activity or expression level of KRAS, and, optionally, EGFR and/or BRAF activity or expression level (and hence the corresponding marker gene expression level(s)) do not change towards their “normal” (expression) level(s) (in a sufficient manner) upon the cancer treatment/therapy. In one specific embodiment, a “non-responder” may be a subject/patient suffering from one of the herein defined resistances.

In context of this embodiment of the invention, one non-limiting example of a (diseased) control subject/patient (responder and/or non-responder) suffering from a cancer defined herein or being prone to suffering from a susceptibility thereto is one having a mutated form of a KRAS gene leading to an aberrant KRAS activity/expression of KRAS.

The skilled person is aware of how a typical/desired response to a known therapy/treatment of a known cancer characterized by the presence of at least one mutation in the KRAS gene (e.g. non-small cell lung cancer, lung adenocarcinoma, pancreatic cancer, colorectal cancer) should proceed or is intended to proceed. Moreover, the skilled person can consider how a typical/desired response to a (unknown) therapy/treatment of a (unknown) cancer characterized by the presence of at least one mutation in the KRAS gene proceeds or is intended to proceed. Based on this knowledge, the means, methods and uses of this invention referring to the efficacy of a therapy/treatment of such a cancer can, for example, also be carried out without employing (a sample of) a particular control subject/patient, i.e. without comparing the “activity of KRAS, and, optionally, EGFR and/or BRAF activity or expression level of at least one marker gene” with a “reference activity of KRAS, and, optionally, EGFR and/or BRAF reference activity or reference expression level determined in (a sample from) a control subject/patient. Simply by comparing the course of the determined “activity of KRAS”, and, optionally, “activity of EGFR” and/or “activity of BRAF” or “expression level of KRAS”, and, optionally, “expression level of EGFR” and/or “expression level of BRAF” during the therapy/treatment of a cancer with the above-mentioned known “typical/desired response”, the skilled person is able to consider about the efficacy of the therapy/treatment monitored/predicted. If the response of a subject/patient is as fast (or even faster) than the “typical/desired response”, the subject/patient is a “responder”. If the response of a subject/patient is slower than the “typical/desired response”, the subject/patient is a “non-responder” (when no substantial response can be seen) or “weak-responder”.

In general, a (desired) efficacy of a treatment of a cancer described herein or susceptibility thereto is indicated/predicted, when the aberrant (i.e. enhanced or decreased) activity or expression level of KRAS, and, optionally, EGFR and/or BRAF activity or expression level of the “marker gene” is shifted back towards the “normal level” of a (healthy) control subject/patient due to/in consequence of said treatment of the cancer or susceptibility thereto.

In context of this invention, the efficacy of a treatment of the cancer defined herein is high, when the subject/patient (to be) treated responds as fast (or even faster) and as complete as a “responder”, i.e. exhibits a “typical/desired response”. This means that said subject/patient reaches the “normal” level of the relevant cytological/haematological parameters and/or KRAS activity (and hence of the corresponding marker gene expression level(s)) of a healthy subject/patient as fast as a “responder”, i.e. in the same manner as in a “typical/desired response”.

In context of this invention, the efficacy of a treatment of the cancer defined herein is moderate/low, when the subject/patient (to be) treated responds not as fast and/or not as complete as a “responder”, i.e. does not exhibit a “typical/desired response”. This means that said subject/patient does not reach the “normal” level of the relevant cytological/haematological parameters and/or activity or expression level of KRAS, and, optionally, the “normal” level of activity or expression level of EGFR and/or of BRAF (and hence of the corresponding marker gene expression level(s)). Accordingly, a moderate/low efficacy means also that the activity/expression level of KRAS, and, optionally, of EGFR and/or of BRAF of a healthy subject/patient is not reached as complete and/or as fast as a “responder”, i.e. not in the same manner as in a “typical/desired response”.

In context of this invention, there is no efficacy of a treatment of the cancer at all, when the subject/patient (to be) treated does not respond at all.

Particularly, when the efficacy of a treatment of the cancer as monitored/predicted in context of this invention is moderate/low or, if there is no such efficacy, a change of the (planned) therapy/treatment might be considered.

In an alternative embodiment, of the herein disclosed means, methods and uses of monitoring/predicting, the reference activity or reference expression level of KRAS, and, optionally, EGFR and/or BRAF/level of a control subject/patient can be replaced by a reference activity of KRAS, and, optionally, EGFR and/or BRAF activity or expression level from the subject/patient to be treated itself obtained prior to (or at the beginning of) the treatment/therapy. In this specific case, the “control subject/patient” would be the subject/patient to be treated itself. The efficacy of the cancer treatment would then be assessed on the basis of how the activity of KRAS, and, optionally, EGFR and/or BRAF activity or expression level of the at least one marker gene in accordance with this invention changes during treatment/therapy compared with said particular reference activity of KRAS, and, optionally, EGFR and/or BRAF activity or expression level. The more significant and/or faster said change is the more efficacious is the treatment/therapy.

As mentioned above, the efficacy of a treatment of the cancer is assessed in accordance with specific embodiments of this invention, on the basis that the activity or the expression level of KRAS, and, optionally. EGFR and/or BRAF activity or expression level of at least one marker gene as described herein is different from a certain reference activity of KRAS, and, optionally, EGFR and/or BRAF activity or expression level. Thereby, it is clear that different means higher or lower, depending on whether the cancer comes along with an up- or down-regulated activity of KRAS, and, optionally, EGFR and/or BRAF activity or expression level.

In this context, different, higher or lower means different, higher or lower than the normal (range of) activity or expression level of KRAS, and, optionally, EGFR and/or BRAF activity or expression level of said at least one marker gene. For example, different, higher or lower means at least 1.5 fold, at least 2 fold, at least 2.5 fold, at least 3 fold, at least 4 fold, at least 5 fold, at least 7 fold, at least 10 fold, at least 15 fold, at least 25 fold, at least 50 fold, at least 100 fold or at least 200 fold different, higher or lower, wherein the higher values are preferred.

If at all, in which direction (i.e. higher or lower), and to which extend the activity or expression level of KRAS, and, optionally, EGFR and/or BRAT activity or expression level of at least one marker gene as described herein differs from its corresponding reference expression level, can easily be deduced by the skilled person based on the teaching provided herein and the common general knowledge. Accordingly, it is possible to assess for each marker gene particularly described herein, whether a given difference between the reference activity or expression level of KRAS, and, optionally, EGFR and/or BRAF activity or expression level and the activity or expression level of KRAS, and, optionally, EGFR and/or BRAF activity or expression level of a subject/patient to be diagnostically assessed is diagnostic for a cancer characterized by the presence of at least one KRAS mutation or the efficacy of a treatment thereof.

As mentioned, in context of the means, methods and uses of monitoring/predicting as disclosed herein, the extent of the “difference” between the activity or expression level of KRAS, and, optionally, EGFR and/or BRAF activity or expression level in a sample or patient to be assessed or monitored in accordance with this invention and corresponding “reference activity/reference expression level” of KRAS, and, optionally, EGFR and/or BRAF “reference activity/reference expression level” is indicative for the (predicted) efficacy of the therapy/treatment of a the cancer (to be) performed.

For example, if the control subject/patient is a “responder” (e.g. a “positive control”), a minimal or low difference of the activity/expression level of KRAS, and, optionally. EGFR and/or BRAF activity or expression level between the “reference” and the sample/patient to be assessed or monitored is indicative for “high efficacy”. The same may be evaluated on the basis of a “typical/desired response”. Also here a low difference (at a certain point in time) to the “control” indicates a high efficacy. Similarly, in case that the “control subject/patient” is a non-responder (e.g. “negative control”) or a patient sample obtained before treatment, a higher difference in reference activity or reference expression level of KRAS, and, optionally, EGFR and/or BRAF reference activity or reference expression level obtained prior to/at the beginning of a therapy/treatment of a cancer characterized by the presence of at least one KRAS mutation may also be indicative for a high efficacy. In this context, a high and thereby “positive” difference (at a certain point in time) between a “non-responder sample” or an “own sample before treatment or at the beginning of the treatment” of the given patient and the sample assessed during or after treatment indicates a high efficacy.

As can be deduced from the above, the reference activity or reference expression level of KRAS, and, optionally, EGFR and/or BRAF reference activity or reference expression level as referred to herein may be taken at the day of diagnosis, once the therapy/treatment is initiated, in between and/or during therapy/treatment, either from the subject/patient to be treated itself or from a corresponding control subject/patient (healthy/responder/non-responder). The reference activity of KRAS, and, optionally, EGFR and/or BRAF reference activity or reference expression level may be determined at the same or at a different point in time than the activity of KRAS, and, optionally. EGFR and/or BRAF activity or expression level of the at least one marker gene, for example with respect to the course of the therapy/treatment.

In particular, if the reference activity or expression level of KRAS, and, optionally, EGFR and/or BRAF activity or expression level is obtained from a control subject/patient different from the subject/patient to be treated, it is preferred that the reference activity or reference expression level of KRAS, and, optionally, EGFR and/or BRAF reference activity or reference expression level is determined at the same point in time during therapy/treatment. In particular, if the reference activity or reference expression level of KRAS, and, optionally, EGFR and/or BRAF reference activity or reference expression level is obtained from the subject/patient to be treated itself, the reference activity or reference expression level of KRAS, and, optionally, EGFR and/or BRAF reference activity or reference expression level should be determined at a different point in time during therapy/treatment to allow comparison, for example, at the beginning of (or prior to) the therapy/treatment.

In general, activity or expression level of KRAS, and, optionally, EGFR and/or BRAF activity or expression level(s) described herein can be determined once or, preferably, several times. For example, activity or expression level of KRAS, and, optionally, EGFR and/or BRAF activity or expression levels can be determined on a daily, weekly, monthly or yearly basis during therapy/treatment. Commonly, the requirements of corresponding studies would be met, if the frequency of determining activity or expression level of KRAS, and, optionally, EGFR and/or BRAF activity or expression levels decreases during process of therapy/treatment. Non-limiting examples of schemes of determining activity or expression level of KRAS, and, optionally, EGFR and/or BRAF activity or expression levels in accordance with this invention are provided herein.

It is of note that the skilled person is readily in the position to elect (a) suitable control patient(s)/subject(s) and the point(s) in time when the (reference) activity or (reference) expression level of KRAS, and, optionally, EGFR and/or BRAF (reference) activity or (reference) expression levels are determined for each individual setup of the means, methods and uses provided.

In one embodiment, the present invention relates to the use of a (transgenic) cell or a (transgenic) non-human animal having at least one mutation in the KRAS gene, and, optionally, in the EGFR gene and/or the BRAF gene for screening and/or validation of a medicament for the treatment of cancer characterized by the presence of at least one mutation in the KRAS gene. The term “cell” as used in this context may also comprise a plurality of cells as well as cells comprised in a tissue. A cell to be used may, for example, be a primary tumor cell. The tumor cell or cell to be used in the screening or validation method may be obtained from samples from a (transgenic) non-human animal suffering from non-small cell lung cancer, lung adenocarcinoma, pancreatic cancer, colorectal cancer, breast cancer, leukemias, prostate cancer, lymphomas, brain tumors, pediatric tumors or sarcomas. The tumor cell or cell may also be obtained from patient samples (e.g. biopsies), in particular a biopsy/biopsies from a patient/subject suffering from non-small cell lung cancer, lung adenocarcinoma, pancreatic cancer, colorectal cancer, breast cancer, leukemias, prostate cancer, lymphomas, brain tumors, pediatric tumors or sarcomas. Accordingly, the tumor cell or cell may be a human tumor cell or cell. Again, such a cell to be used in the present screening or validation methods may be comprised in a tissue or tissue sample, like in a sample biopsy.

The used non-human animal or cell may be transgenic or non transgenic. “Transgenic” in this context particularly means that at least one of the marker gene as described or defined herein is over- or under-expressed. For example, if an HSP90-inhibitor is to be screened and/or validated, it is preferred that such marker genes are over-expressed, the activity or expression level of KRAS, and, optionally. EGFR and/or BRAF activity or expression level of which is enhanced when the activity or expression level of KRAS, and, optionally, EGFR and/or BRAF activity or expression level is enhanced and/or that such marker genes are under-expressed, the activity or expression level of KRAS, and, optionally, EGFR and/or BRAF activity or expression level of which is decreased when the activity or expression level of KRAS, and, optionally. EGFR and/or BRAF activity or expression level is enhanced.

“Transgenic” in this context may also mean that KRAS is over- or under-expressed, and/or that the KRAS-activity in the transgenic non-human animal or a transgenic cell is enhanced or decreased. A preferred (transgenic) non-human animal or (transgenic) cell in context of the invention suffers from a cancer characterized by the presence of a mutation in the KRAS gene, particularly from such cancer for the treatment of which the medicament is to be screened and/or validated. For example, if a medicament for non-small lung cancer is to be screened and/or validated, the (transgenic) non-human animal or (transgenic) cell is particularly intended to suffer from non-small lung cancer. i.e. to have, for example, an increased KRAS activity and/or increased expression level.

The term “transgenic non-human animal” or “transgenic cell” as used herein refers to an non-human animal or cell, not being a human, that comprises genetic material different from the genetic material of a corresponding wild-type animal/cell. “Genetic material” in this context may be any kind of a nucleic acid molecule, or analogues thereof, for example a nucleic acid molecule, or analogues thereof as defined herein. “Different” in this context means additional or fewer genetic material with respect to the genome of the wild-type animal/cell and/or rearranged genetic material, i.e. genetic material present at a different locus of the genome with respect to the genome of the wild-type animal/cell. An overview of examples of different expression systems to be used for generating transgenic cell/animal is, for instance, contained in Methods in Enzymology 153 (1987), 385-516, in Bitter et al. (Methods in Enzymology 153 (1987), 516-544) and in Sawers et al. (Applied Microbiology and Biotechnology 46 (1996). 1-9). Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440).

In a preferred embodiment, the (transgenic) non-human animal or (transgenic) cell is or is derived from a mammal. Non-limiting examples of the (transgenic) non-human animal or derived (transgenic) cell are selected from the group consisting of a mouse, a rat, a rabbit, a guinea pig and a Drosophila.

Preferably, the (transgenic) cell in accordance with this invention may be an animal cell, for example, a non-human animal cell. However, also human cells are envisaged to be employed as cells in context of the present invention. In a non limiting example, such cell may be an embryonic stem cell (ES cell), particularly a non-human animal ES, like, for example, a mouse or rat ES cell. The (transgenic) cell as described herein, particularly the ES cell, may also be used for generating the (transgenic) non-human animal as described herein. The ES cell technology for generating transgenic animals is well known in the art and for example is described in Pirity (Methods Cell Biol, 1998, 57:279).

Generally, the (transgenic) cell may be a prokaryotic or eukaryotic cell. For example, the (transgenic) cell may be a bacterial, yeast, fungus, plant or animal cell. In general, the transformation or genetically engineering of a cell with a nucleic acid construct or vector can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990.

The (transgenic) non-human animal or (transgenic) cell as described or defined in context of this invention is particularly useful in methods for screening and/or validation of a medicament for the treatment of cancers as defined and described herein.

These screening methods may, in particular, performed in vivo using, for example, (transgenic) animals as described herein (e.g. rats, mice and the like) and/or animals comprising (a) cell(s), (a) tissue(s) or (a) cell culture(s) characterized by at least one mutation in the KRAS gene and, optionally, in the EGFR and/or the BRAF gene. Said (a) cell(s), (a) tissue(s) or (a) cell culture(s) may, for example, be obtained/derived from (a) tumor cell(s)/tumor(s) characterized by at least one mutation in the KRAS gene and, optionally, in the EGFR and/or the BRAF gene. In particular, said (a) cell(s), (a) tissue(s) or (a) cell culture(s) may be obtained from a subject/patient suffering from a cancer characterized by at least one mutation in the KRAS gene and, optionally, in the EGFR and/or the BRAF gene. Exemplary cancers described herein are, inter alia, non-small lung cancer, pancreatic cancer, colorectal cancer, breast cancer and leukemia. These in vivo screening methods may in particular comprise measuring and determining differences in tumor volume, for example, in the (transgenic) animals described herein above.

Accordingly, the present invention also relates to such a method for screening and/or validation of a medicament for the treatment of a cancer. Said method comprising the steps of

a) administering to a (transgenic) non-human animal or (transgenic) cell as defined herein said medicament to be screened/validated;
b) determining in (a sample from) said animal or cell the activity or expression level of KRAS, and, optionally, EGFR and/or BRAF activity or expression level in accordance with this invention;
c) comparing the activity or expression level determined in h) with a reference activity or reference expression level of KRAS, and, optionally, EGFR and/or BRAF activity or expression level, determined in (a sample from) a control (transgenic) non-human animal or (transgenic) cell as defined herein to which said medicament to be screened has not been administered; and
d) selecting said medicament when said activity or expression level or activity of KRAS, and, optionally, EGFR, and/or BRAF activity or expression level determined in b) differs from said reference expression level or reference activity determined in c).

The corresponding definitions and descriptions provided above, for example with respect to “marker gene”, “therapy/treatment”, “efficacy”, “cancer characterized by the presence of at least one mutation in the KRAS gene” or “susceptibility” thereto, “(control) subject/patient”, “(transgenic) non-human anima or “(transgenic) cell”, “expression level”, “reference expression level” etc., apply here, mutatis mutandis. Particularly the relevant definitions and descriptions provided above with respect to “control subject/patient” also apply to the “control (transgenic) non-human animal” or “(transgenic) cell”, mutatis mutandis.

In context of this invention, “screening and/or validation of medicaments” means, on the one hand, whether a given set of compounds comprises one or more compound(s) that can function as (a) medicament(s), and/or, on the other hand, whether (a) given compounds) can function as (a) medicament(s). It is particularly intended that the medicaments to be screened and/or validated in context of this invention are medicaments for the treatment, prevention and/or amelioration of a cancer as defined herein.

The skilled person is readily in the position to put this embodiment of the present invention into practice. For example, by doing so, the compound(s)/medicament(s) to be screened and/or validated may be administered to the non-human (transgenic) animal or cell described herein, and, afterwards (for example after a certain period of time sufficient to allow a compound to effect on a cancer as described herein), it is analyzed whether the cancer, or a symptom thereof, of said animal/cell is ameliorated.

The present invention also relates to a kit for carrying out the methods or uses of this invention. In a preferred embodiment, said kit useful for carrying out the methods and uses described herein comprises oligonucleotides or polynucleotides capable of determining the presence of at least one mutation in the KRAS gene, and, optionally, the EGFR, and/or the BRAF gene.

For example, said kit may comprise (a) compound(s) required for specifically determining the activity or expression level of KRAS, and, optionally, of EGFR and/or of BRAF activity or expression level as defined herein. Moreover, the present invention also relates to the use of (a) compound(s) required for specifically determining the activity or expression level of KRAS, and, optionally, EGFR and/or BRAF activity or expression level as defined herein for the preparation of a kit for carrying out the methods or uses of this invention. On the basis of the teaching of this invention, the skilled person knows which compound(s) is (are) required for specifically determining the activity of KRAS, and, optionally, of EGFR and/or of BRAF activity or expression level as defined herein. For example, such compound(s) may be (a) “binding molecule(s)”, like, for example, (a) “binding molecule(s)” as defined herein-above. Particularly, such compounds) may be (a) (nucleotide) probe(s), (a) primer(s) (pair(s)), (an) antibody(ies) and/or (an) aptamer(s) specific for at least one marker gene as described herein or for a product thereof. In a preferred embodiment, the kit (to be prepared in context) of this invention is a diagnostic kit.

In a particularly preferred embodiment of the present invention, the kit (to be prepared in context) of this invention or the methods and uses of the invention may further comprise or be provided with (an) instruction manual(s). For example, said instruction manual(s) may guide the skilled person (how) to determine the (reference) activity or (reference) expression level of KRAS, and, optionally, of EGFR and/or of BRAF, i.e. (how) to diagnose the cancer described herein or a susceptibility thereto, (how) to monitor the efficacy of a treatment of said cancer or a susceptibility thereto or (how) to predict the efficacy of a treatment of said cancer or a susceptibility thereto in accordance with the present invention. Particularly, said instruction manual(s) may comprise guidance to use or apply the herein provided methods or uses.

The kit (to be prepared in context) of this invention may further comprise substances/chemicals and/or equipment suitable/required for carrying out the methods and uses of this invention. For example, such substances/chemicals and/or equipment are solvents, diluents and/or buffers for stabilizing and/or storing (a) compound(s) required for specifically determining the activity or expression level of KRAS, and, optionally, EGFR and/or BRAF activity or expression level.

In one embodiment the present invention relates to the use of an HSP90 inhibitor as defined herein above for the preparation of a pharmaceutical composition for the treatment, amelioration and/or prevention of treatment of cancer characterized by the presence of at least one activating mutation in the KRAS gene. Alternatively, the present invention may relate to an HSP90 inhibitor as defined herein for use in treating, ameliorating and/or preventing cancer characterized by the presence of at least one activating mutation in the KRAS gene. Accordingly, the present invention relates to a method for preventing, treating or ameliorating a cancer characterized by the presence of at least one activating mutation in the KRAS gene comprising the administration of an effective amount of the HSP90 inhibitor as defined herein above to a subject in need of such a prevention, treatment or amelioration. Preferably, the subject in need of such a prevention, treatment or amelioration is a human.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

The skilled person knows that the effective amount of pharmaceutical composition administered to an individual will, inter alia, depend on the nature of the compound. For example, if said compound is a HSP90 inhibitor as described herein above the total pharmaceutically effective amount of pharmaceutical composition administered parenterally per dose will be in the range of about 1 μg HSP90 inhibitor/kg/day to 10 mg HSP90 inhibitor/kg/day of patient body weight, although, as noted above, this will be subject to therapeutic discretion. More preferably, this dose is at least 0.01 mg HSP90 inhibitor/kg/day and most preferably for humans between about 0.01 and 10 mg HSP90 inhibitor/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

Pharmaceutical compositions of the invention may be administered orally, rectally, parenterally, intracisternally, intravaginally, intraperitoneally, topically (as by powders, ointments, drops or transdermal patch), bucally, or as an oral or nasal spray.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or microcapsules, Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3.218.121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g. 0.2 micron membranes). Therapeutic components of the pharmaceutical composition generally are placed into a container having a sterile access port, for example, an intravenous solution hag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

The present invention also relates to a combination of cell lines selected from the group consisting of A427, A549, Calu-1, Calu-3, Calu-6, H1299, H1355, H1395. H1437, H1563, H1568, H1648, H1650, H1666, H1734, H1755, H1770, H1781, H1792, H1819, H1838, H1915, H1944, H1975, H1993, H2009, H2030, H2052, H2087, H2110, H2122, H2126, H2172, H2228, H23, H2347, H2444, H28, H358, H441, H460, H520, H522, H596, H647, H661, H820, HCC2935, HCC4006, HCC827, SK-LU-1, EKVX, H322M, HOP-62, HOP-92, Colo699, DV-90, HCC15, HCC366, HCC44, HCC78, LCLC103H, LCLC97TM1 and LouNH91.

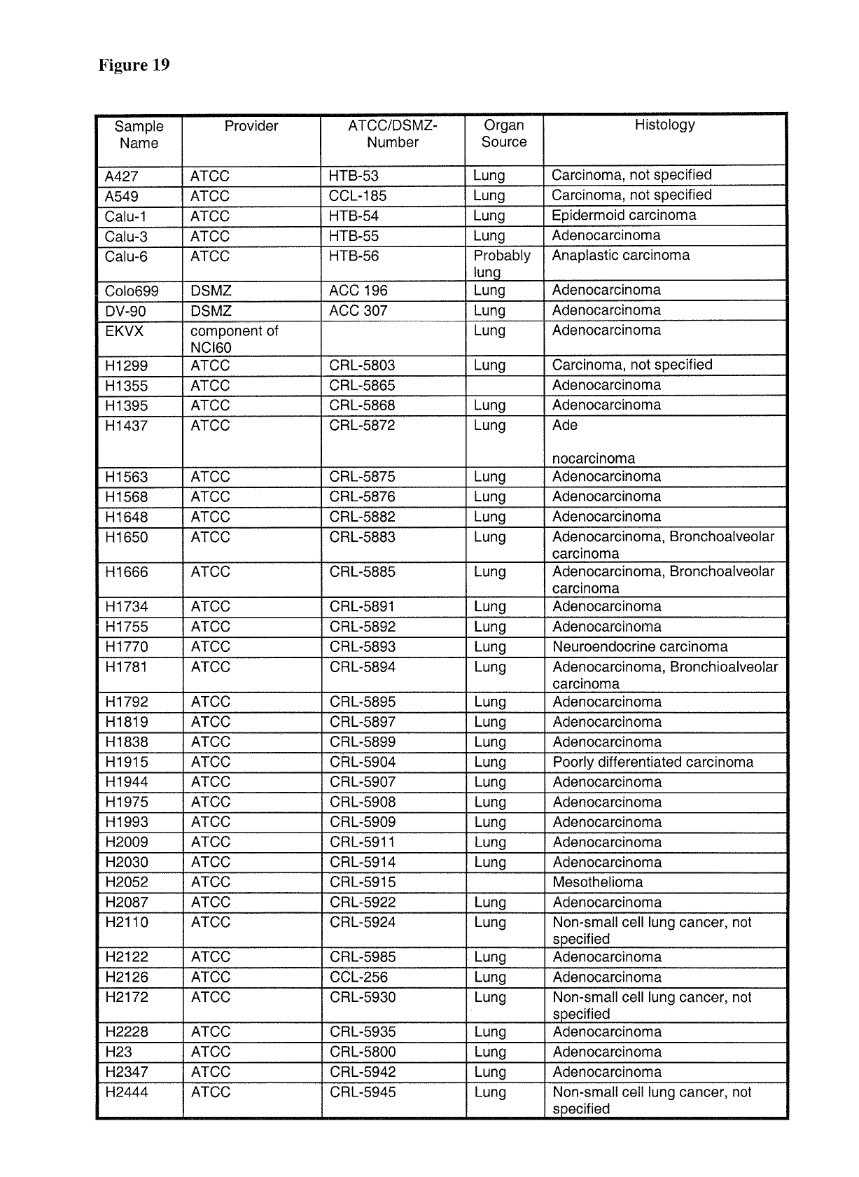

These combinations of cell lines should comprise at least 3, 4, 5, 6, 7, 8, 9, 10, 11, 12, 13, 14, 15, 20, 25, 30, 35, 40, 45, 50, 55 or 60 of the cell lines as provided herein above. In particular the combination of cell lines should comprise at least 60 cell lines as provided herein above. These cell lines, and in particular their combination are particular useful as model systems for the assessment of any potential drug susceptibility. This usefulness of these specifically selected cell lines (in combination) for the assessment of drug susceptibility is demonstrated in the appended example. All of the mentioned cell lines are available to the person skilled in the art and the public from cell depositary institutions, in particular ATCC or DMSZ as illustrated in FIG. 19 where also corresponding accession numbers for these cells are provided.

Also the use of the combination of cell lines as defined herein above for predicting susceptibility to a drug, in particular, an HSP90 inhibitor, is disclosed herein. The combination of cell lines may be useful for predicting the susceptibility to a drug, in particular to an HSP90 inhibitor or responsiveness of a (mammalian) tumor cell to treatment with a drug, in particular an HSP90 inhibitor. It may also be useful in predicting whether a patient is likely to respond to or is sensitive to a drug, in particular an HSP90 inhibitor. Corresponding means and methods for predicting susceptibility/responsiveness and the like are well known in the art and also described herein above. Accordingly, a skilled person will know how to use such a combination of cell lines in this context.

The present invention is further described by reference to the following non-limiting figures and examples.

In summary, the present invention relates to the following items described herein in detail and also reflected in the appended claims:

1. A method of selecting (a) cell(s), (a) tissue(s) or (a) cell culture(s) with susceptibility to an HSP90 inhibitor, comprising the steps:
(a) determining the presence of at least one activating mutation in the KRAS gene in said cell, tissue or cell culture; and
(b) selecting (a) cell(s), tissue(s) or cell culture(s) with at least one activating mutation in the KRAS gene.
   2. The method of item 1, further comprising the steps
(i) contacting said cell(s), tissue(s) or cell culture(s) with an HSP90 inhibitor; and
(ii) evaluating viability of said cell(s), tissue(s) or cell culture(s) contacted with an HSP90 inhibitor.
   3. A method for determining the responsiveness of a mammalian tumor cell or cancer cell to treatment with an HSP90 inhibitor, said method comprising determining the presence of at least one activating mutation in the KRAS gene in said tumor cell, wherein said activating mutation is indicative of whether the cell is likely to respond or is responsive to the treatment.
   4. The method of any one of items 1 to 3, whereby additionally and/or optionally also an activating mutation in the EGFR and/or the BRAF gene is determined.
   5. In vitro method for the identification of a responder for or a patient sensitive to an HSP90 inhibitor, said method comprising the following steps:
(a) obtaining a sample from a patient suspected to suffer from or being prone to suffer from a cancer characterized by the presence of at least one activating mutation in the KRAS gene, and, optionally, in the EGFR and/or the BRAF gene; and
(b) evaluating the presence of at least one activating mutation in the KRAS gene, and, optionally, the EGFR and/or the BRAF gene;
whereby an activating mutation in the KRAS gene alone or in addition to an activating mutation in the EGFR and/or the BRAF gene is indicative for a responding patient or is indicative for a sensitivity of said patient to an HSP90 inhibitor.
   6. The method of any one of items 1 to 5, wherein said HSP90 inhibitor is geldanamycin or a derivative thereof.
   7. The method of item 6, wherein said geldanamycin derivative is 17-AAG or IPI-504.
   8. The method of any one of items 1 to 5, wherein said HSP90 inhibitor is NVP-AUY922.
   9. The method of any one of items 1 to 8, wherein said mutation in the KRAS gene is selected from the group consisting of KRAS_G12C, KRAS_G12R, KRAS_G12D, KRAS_G12A, KRAS_G12S. KRAS_G12V, KRAS_G13D. KRAS_G13S. KRAS_G13C, KRAS_G13V, KRAS_Q61H, KRAS_Q61R, KRAS_Q61P, KRAS_Q61L, KRAS_Q61K, KRAS_Q61E, KRAS_A59T and KRAS_G12F.
   10. The method of any one of items 4 to 9, wherein said mutation in the EGFR gene is selected from the group consisting of EGFR_D770_N771>AGG; EGFR_D770_N771insG; EGFR_D770N771insG; EGFR_D770N771insN; EGFR_E709A; EGFR_E709G; EGFR_E709H; EGFR_E709K: EGFR_E709V; EGFR_E746 A750del: EGFR_E746 A750del; T751A; EGFR_E746 A750del, V ins; EGFR_E746 T751del, I ins; EGFR_E746 T751del, S752A; EGFR_E746 T751del, S752D; EGFR_E746T751del, V ins; EGFR_G719A; EGFR_G719C: EGFR_G719S; EGFR_H773V774insH; EGFR_H773_V774insNPH; EGFR_F1773_V774insPH; EGFR_H773>NPY; EGFR_L747_E749del; EGFR_L747_E749del, A750P; EGFR_L747 S752del; EGFR_L747_S752del, P753S; EGFR_L747_S752del, Q ins; EGFR_L747_T750del, P ins; EGFR_L747_T751del; EGFR_L858R; EGFR_L861Q; EGFR_M766_A767insAI; EGFR_P772_H773insV; EGFR_S752_I759del; EGFR_S768I; EGFR_T790M; EGFR_V769_D770insASV: EGFR_V769_D770insASV; and EGFR_V774_C775insHV.
   11. The method of any one of items 4 to 10, wherein said mutation in the BRAF gene is selected from the group consisting of BRAF_BRAF_D594V, BRAF_F468C, BRAF_F595L, BRAF_G464E, BRAF_G464R, BRAF_G464V, BRAF_G466A, BRAF_G466E, BRAF_G466R, BRAF_G466V, BRAF_G469A, BRAF_G469E, BRAF_G469R, BRAF_G469R, BRAF_G469S, BRAF_G469V, BRAF_G596R, BRAF_K601E, BRAF_K601N, BRAF_L597Q, BRAF_L597R, BRAF_L597S, BRAF_L597V, BRAF_T599I, BRAF_V600E, BRAF_V600K, BRAF_V600L, and BRAF_V600R
   12. The method of any one of items 4 to 11, wherein said mutation in the KRAS gene, the EGFR and/or the BRAF gene is detected by SSP, PCR-RFLP assay, real-time PCR, sequencing, HPLC or mass-spectrometric genotyping.
   13. The method of any one of items 5 to 12, wherein said sample is obtained from a patient suspected to suffer from or being prone to suffer from cancer.
   14. Use of an oligo- or polynucleotide capable of detecting (an) activating mutation(s) of at least one activating mutation in the KRAS gene and, optionally in the EGFR and/or in the BRAF gene for diagnosing sensitivity to an HSP90 inhibitor as defined in any one of items 6 to 8.
   15. The use of item 14, wherein said oligonucleotide is about 15 to 100 nucleotides in length.
   16. A method of monitoring the efficacy of a treatment of a cancer characterized by the presence of at least one activating mutation in the KRAS gene, and, optionally, in the EGFR and/or the BRAF gene in a subject/patient suffering from said disorder or being prone to suffering from said disorder comprising the steps:
a) determining in a cell or tissue sample obtained from said subject/patient the expression or activity of KRAS, and, optionally the activity or expression level of EGFR and/or the activity or expression level of BRAF; and
b) comparing the activity of said at least one marker gene determined in a) with a reference or control expression level or reference or control activity of KRAS, and, optionally with a reference or control expression level of EGFR and/or with a reference or control expression level of BRAF,
wherein the extent of the difference between said activity or expression level determined in a) and said reference expression level or reference activity is indicative for said efficacy of a treatment of said cancer.
   17. A method of predicting the efficacy of a treatment of a cancer characterized by the presence of at least one activating mutation in the KRAS gene, and, optionally, in the EGFR and/or the BRAF gene for a subject/patient suffering from said disorder or being prone to suffering from said disorder comprising the steps:
a) determining in a cell or tissue sample obtained from said subject/patient the activity or expression level of at least one marker gene selected from the group consisting of KRAS. EGFR and/or BRAF; and
b) comparing the activity of said at least one marker gene determined in a) with a reference activity or reference expression level of said at least one marker gene, optionally determined in a cell or tissue sample obtained from a control subject/patient (responder and/or non-responder),
wherein the extent of the difference between said activity or expression level determined in a) and said reference or control activity or said reference or control expression level is indicative for the predicted efficacy of a treatment of cancer.
   18. The method of item 16 or 17, wherein said treatment of cancer characterized by the presence of at least one activating mutation in the KRAS gene, and, optionally, in the EGFR and/or the BRAF gene comprises the administration of an HSP90 inhibitor as defined in any one of items 6 to 8.
   19. Use of a (transgenic) cell or a (transgenic) non-human animal having at least one activating mutation in the KRAS gene, and, optionally, in the EGFR and/or the BRAF gene for screening and/or validation of a medicament for the treatment of cancer characterized by the presence of at least one activating mutation in the KRAS gene.
   20. The method of any of items 5 to 19, wherein said cancer is selected from the group consisting of non-small cell lung cancer, lung adenocarcinoma, pancreatic cancer, colorectal cancer, breast cancer, head and neck cancer, ovarian cancer, endometrial cancer, gastrointestinal cancer (including gastric and esophageal cancer), renal cell cancer, urinary tract carcinomas, leukemias, prostate cancer, lymphomas, melanomas, brain tumors, pediatric tumors and sarcomas.
   21. A kit useful for carrying out the method of any one of items of 1 to 12, 16, 17 and 20, comprising oligonucleotides or polynucleotides capable of determining the presence of at least one activating mutation in the KRAS gene, and, optionally, the EGFR gene and/or the BRAF gene.
   22. Use of an HSP90 inhibitor as defined in any one of items 6 to 8 for the preparation of a pharmaceutical composition for the treatment, amelioration and/or prevention of treatment of cancer characterized by the presence of at least one activating mutation in the KRAS gene.
   23. An HSP90 inhibitor as defined in any one of items 6 to 8 for use in treating, ameliorating and/or preventing cancer characterized by the presence of at least one activating mutation in the KRAS gene.
   24. A combination of cell lines selected from the group consisting of A427, A549, Calu-1, Calu-3, Calu-6, H1299, H1355, H1395, H1437, H1563, H1568, H1648, H1650, H1666, H1734, H1755, H1770, H1781, H1792, H1819, H1838, H1915, H1944, H1975, H1993, H2009, H2030, H2052, H2087, H2110, H2122, H2126, H2172, H2228, H23, H2347, H2444, H28, H358, H441, H460, H520, H522, H596, H647, H661, H820, HCC2935, HCC4006, HCC827, SK-LU-1, EKVX, H322M, HOP-62. HOP-92, Colo699, DV-90, HCC15, HCC366, HCC44, HCC78, LCLC103H. LCLC97TM1 and LouNH91.
   25. Use of the combination of cell lines as defined in item 24 for predicting susceptibility to a drug.
   26. The use of item 25, wherein said drug is an HSP90 inhibitor.

The Figures show:

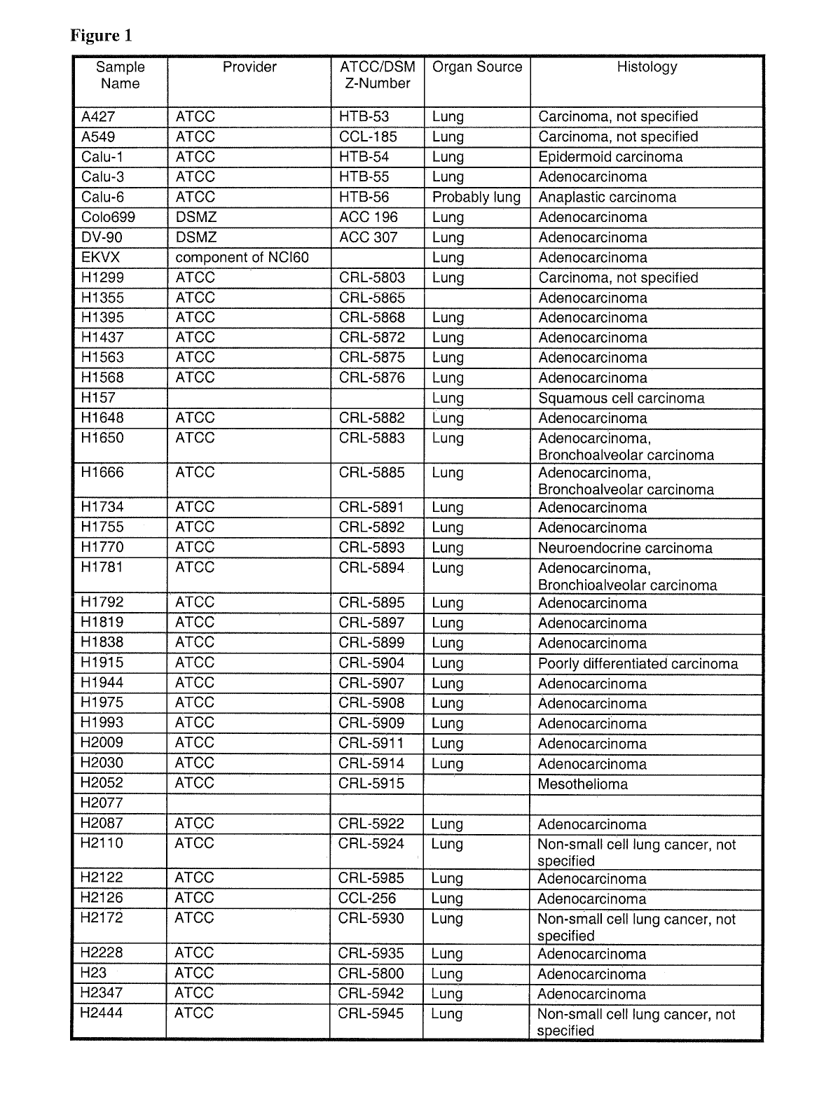

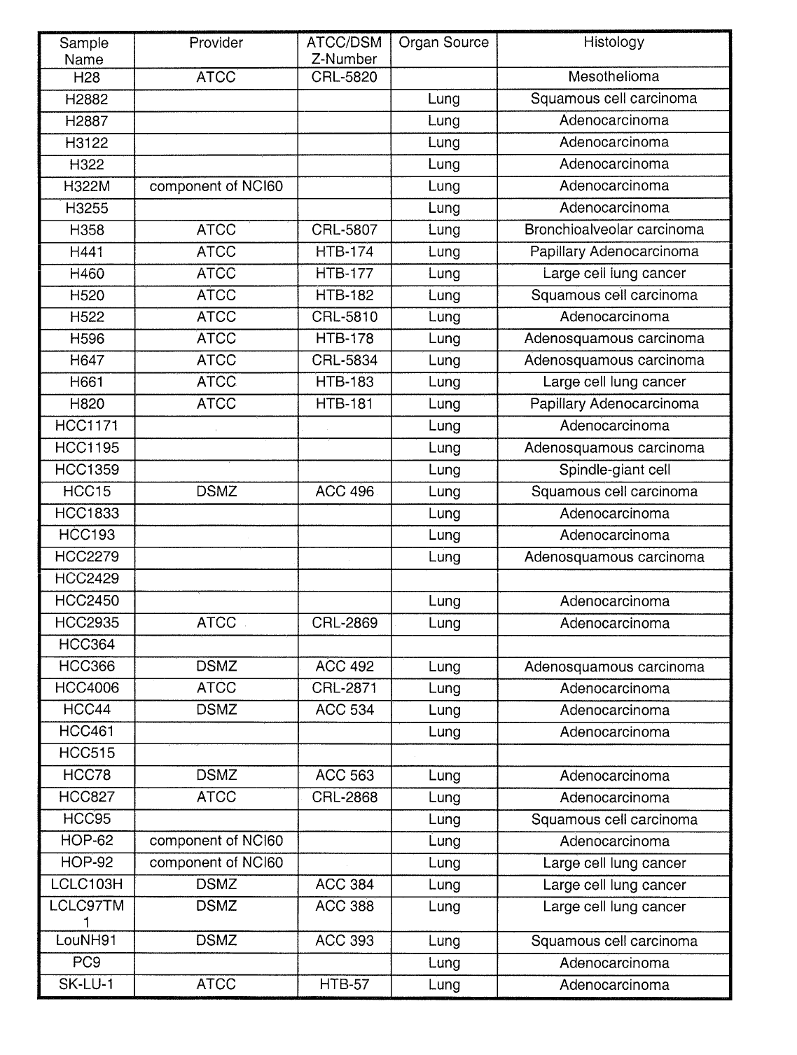

FIG. 1. The NSCLC Cell Line Collection

Overview over the NSCLC cell line collection used in the study including providers and morphopathological details.

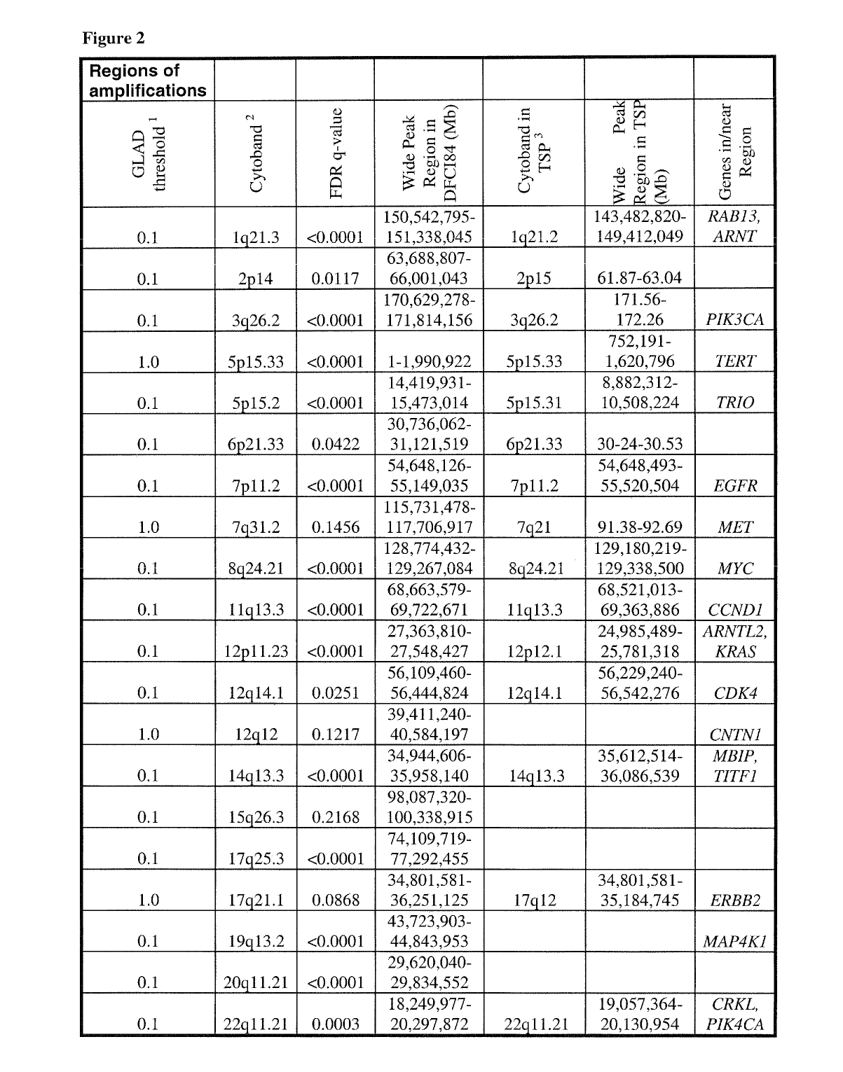

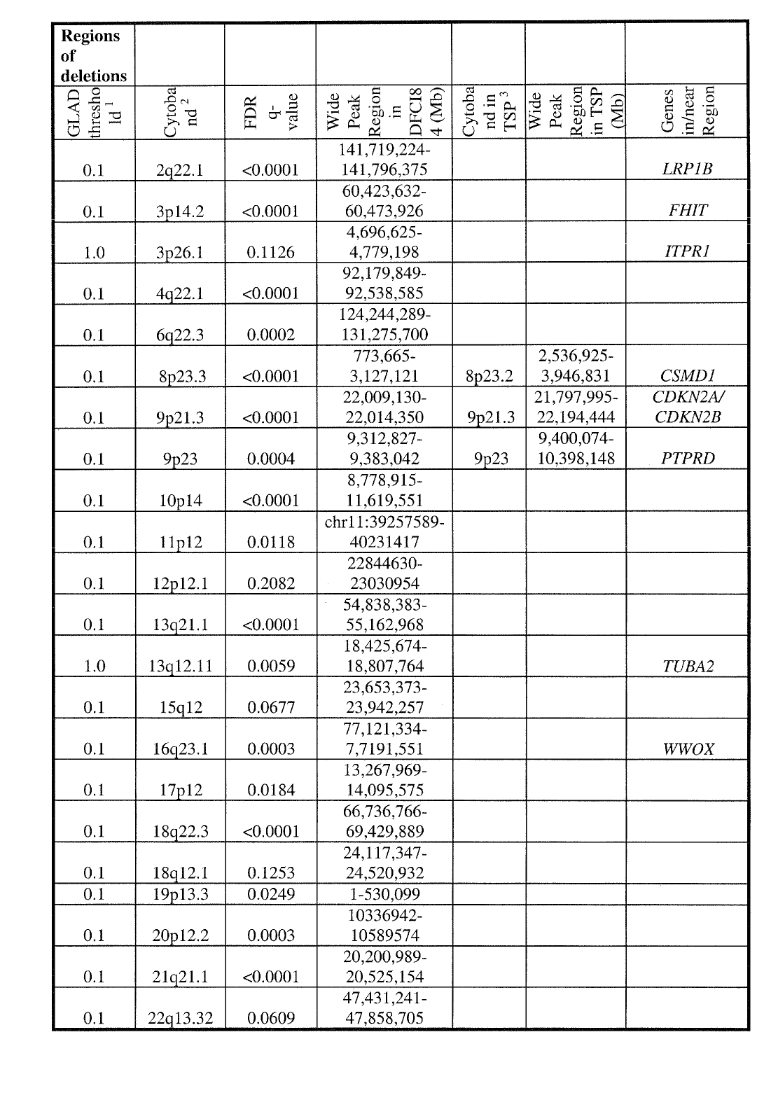

FIG. 2. Significant Lesions in Lung Cancer

Summary of regions with significant copy number alterations as defined by GISTIC in the cell line panel and a primary lung cancer panel.

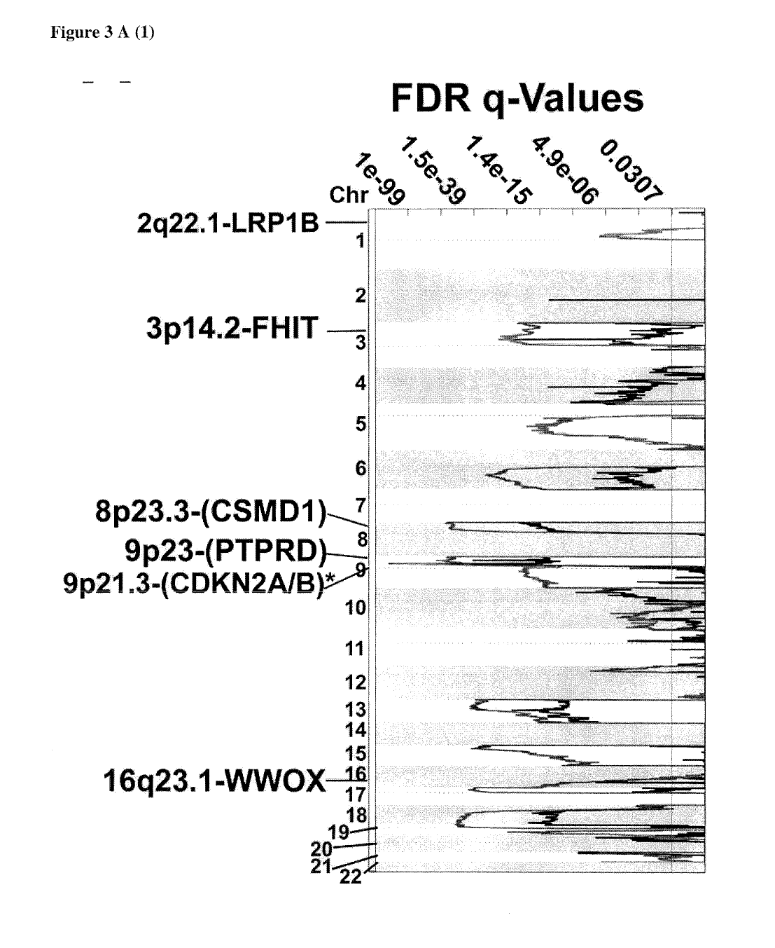

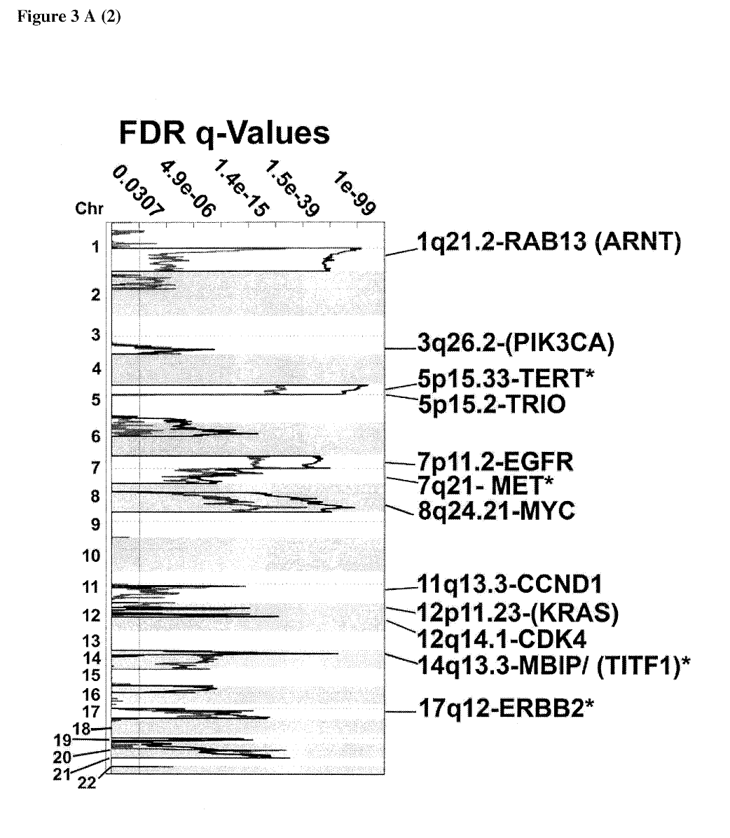

FIG. 3. Genomic Validation of 84 NSCLC Cell Lines

(A) Chromosomal copy number changes of NSCLC cell lines are plotted against those of 371 primary NSCLC tumors. The q-values of the false discovery rates for each alteration (x-axis) are plotted at each genome position (y-axis). FIG. 3A1, chromosomal losses (cell lines, grey; primary tumors, black); FIG. 3A2 right panel, chromosomal gains (cell lines, greycurves; primary tumors, blackcurves). Genomic positions corresponding to even-numbered chromosomes are shaded; dotted lines indicate centromeres; light grey line, q-value cutoff (0.25) for significance. Genes marked in bold represent known targets of mutation in lung adenocarcinoma. Putative targets near peaks are given in parentheses. Genes identified by GISTIC using stringent filtering criteria for peak border detection are marked by asterisks. (B) Oncogene mutations present in NSCLC cell lines (black bars) are plotted according to their relative frequencies in comparison to primary lung tumors (empty bars) (Aviel-Ronen et al., 2006; Bamford et al., 2004; Sharma et al., 2007; Thomas et al., 2007). (C) Hierarchical clustering of significant lesions defined by GISTIC and oncogene mutations using the reciprocal co-occurance ratio as distance measure and average linkage of clusters. The distance of the clusters is reflected in the length of the branches. Note that mutations in EGFR and KRAS occur in a mutually exclusive fashion, while EGFR mutation and amplification are found in the same cluster. (D) Transcriptional profiles from renal primary tissue (grey) and cell lines (black); lung cancer primary tissue (dark grey) and cell lines (light black); lymphoma primary tissue (light black) and lymphoma cell lines (light grey) were analyzed by hierarchical clustering. To reduce noise, probesets were filtered prior to clustering (Coefficient of variation from 1.0-10.0, present call rate 20%; absolute expression >100 in >20% of samples).

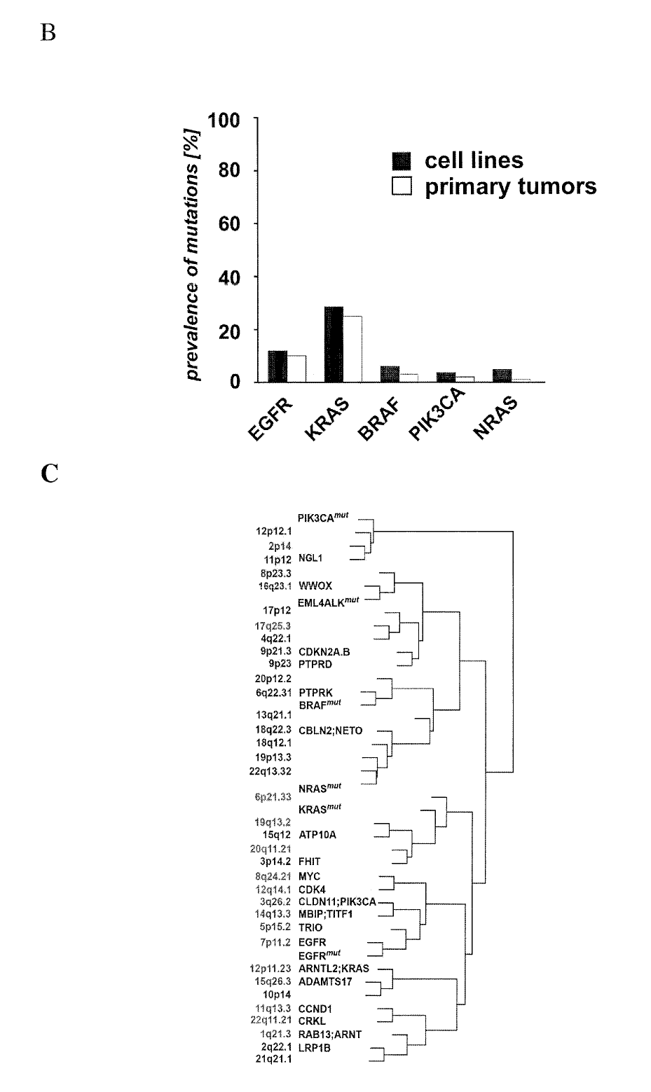

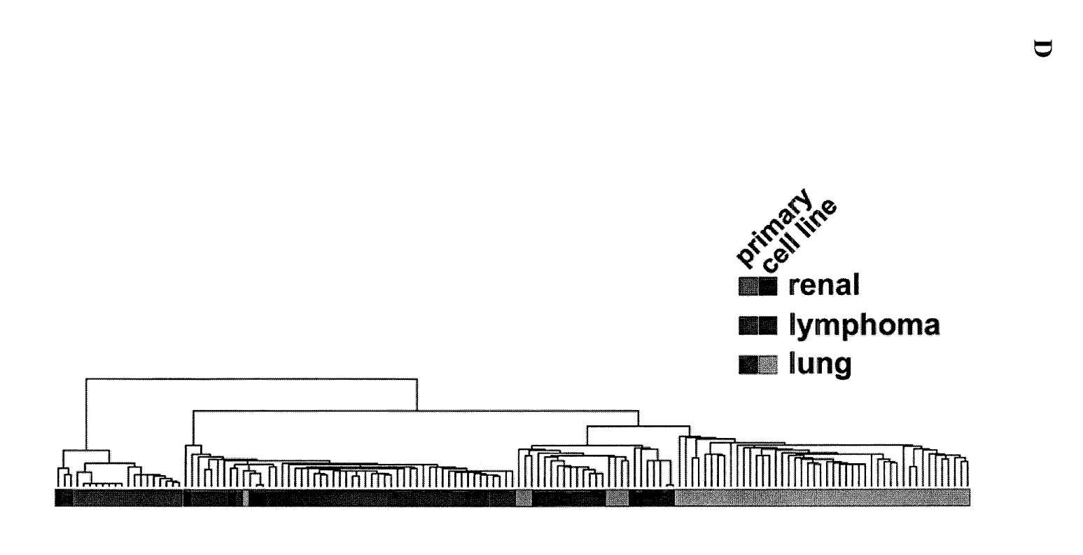

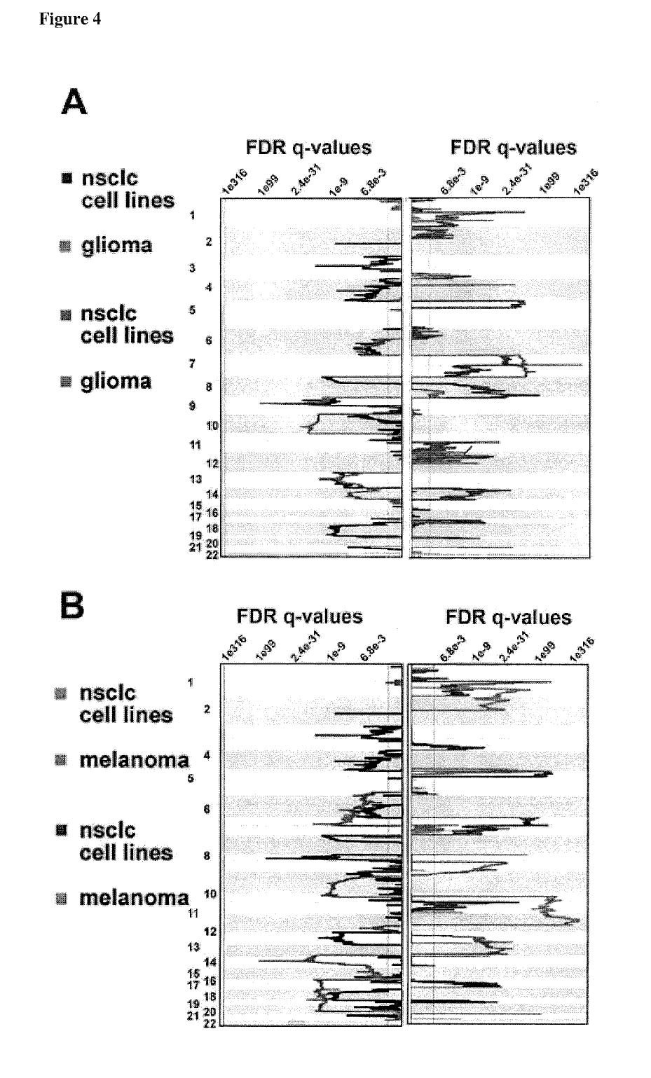

FIG. 4. Profiles of Aberrations in Glioma, Melanoma and Lung Cancer

(A) Chromosomal copy number changes of NSCLC cell lines are plotted against those of primary gliomas. Two separate figures are given for deletions (left panel, NSCLC cell lines in black, gliomas in grey) and amplifications (right panel. NSCLC cell lines in light black, gliomas in dark grey). (B) Chromosomal copy number changes of NSCLC cell lines are plotted against those of primary melanomas (NSCLC cell lines in red respectively blue as above, melanoma short term cultures in purple). (C) Genomic similarity was analyzed by computing correlations of GISTIC q-values for each SNP between NSCLC cell lines and the indicated cancer entity primary lung cancer, ovarian cancer, glioma, melanoma cell culture samples, normal tissues and a randomly split subset of NSCLC cell lines.

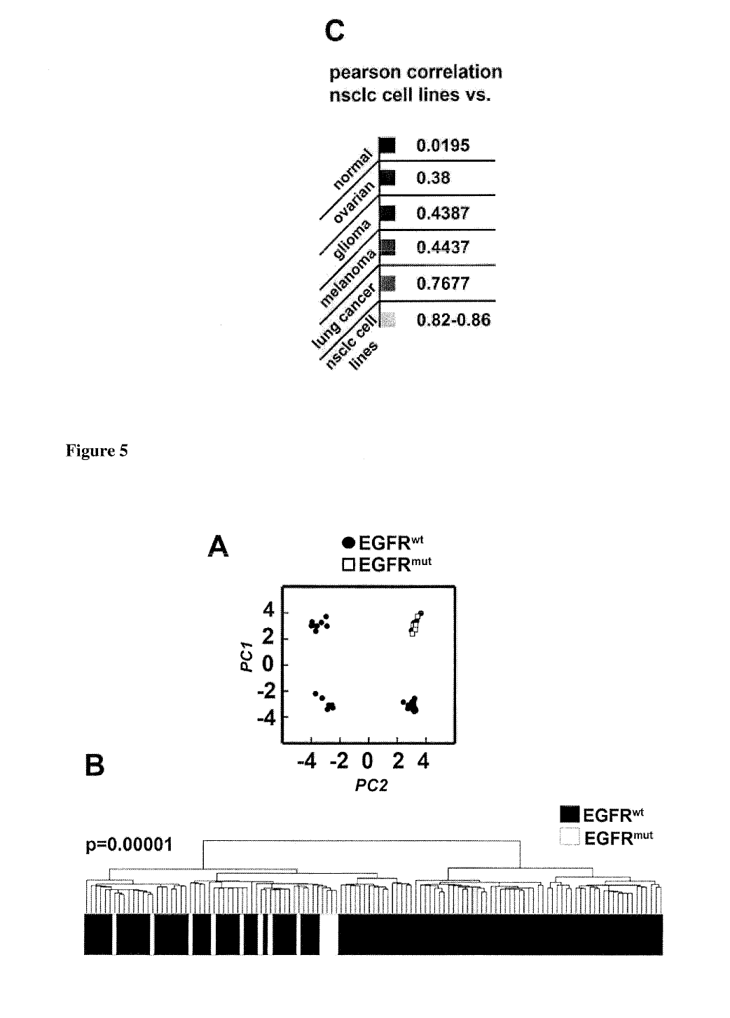

FIG. 5. Robustness of Phenotypic Properties of Mutated EGFR Lung Cancer Cells In Vivo

(A) EGFR mutations induce substantial changes in gene expression as measured by principal component analysis. The first two principal components clearly distinguish cell lines with mutated (mt) EGFR (white squares) and wildtype EGFR (wt) (black dots) (PC; total 54; cumulative variance of first 2 principal components=24.1: %; log₁₀|eigenvector| is given). (B) The signature of EGFR-mutated cell lines (Fold change >2; absolute difference >FC2; 100, p<0.01) was used for hierarchical clustering of 123 primary adenocarcinomas (Bhattacharjee et al., 2001) annotated for the presence (EGFR mt) or absence (EGFR wt) of EGFR mutations. All EGFR-mutant samples were grouped within one cluster. (C) All 123 primary adenocarcinomas with known EGFR mutation status were grouped using RNA transcripts identified in the cell line panel to be differentially expressed between EGFR mutated (mt) and EGFR wildtype (wt) cell lines. EGFR mutant tumors were excluded from survival analyses. (D) The association between presence (amplification, green; mutation, red: deletion, yellow) of genetic lesions to erlotinib sensitivity was analyzed by a t-test and Fisher's exact test. Only EGFR mutations and amplifications can be considered statistically significant if common methods of p-value adjustment are applied (data not shown).

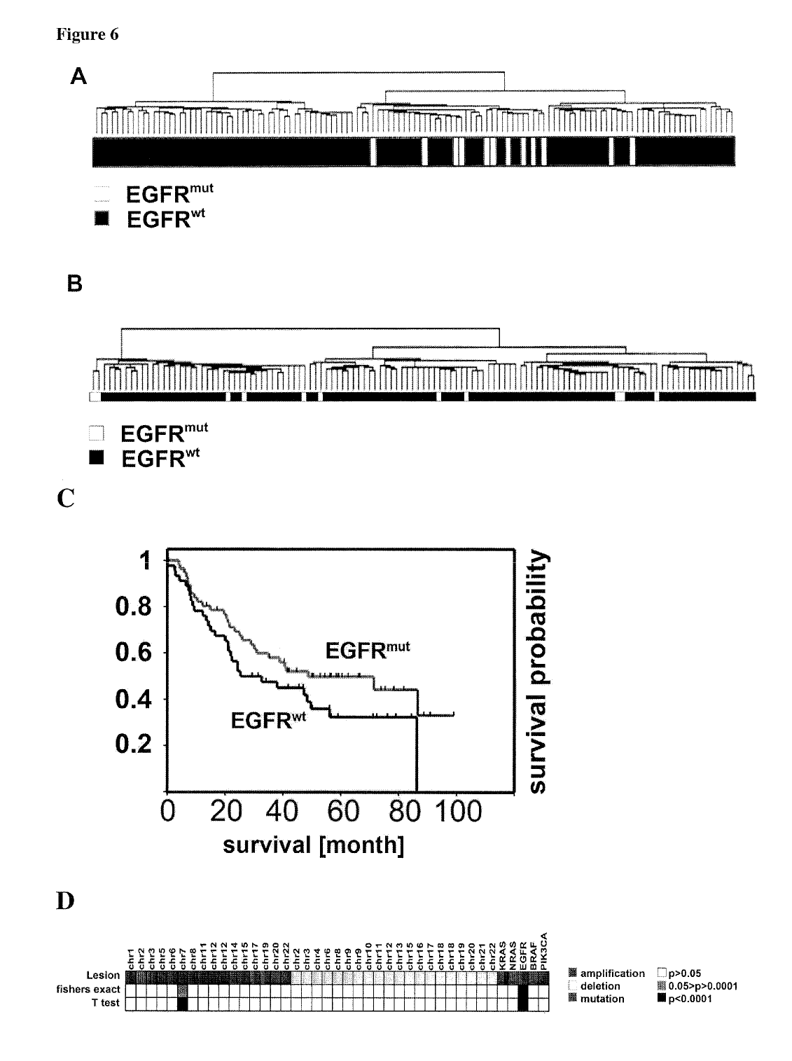

FIG. 6. Phenotypic Properties of Primary Tumors are Conserved in Erlotinib-Sensitive Lung Cancer Cells.

(A) Hierarchical clustering of primary lung adenocarcinomas was performed using genes identified as being differentially expressed in erlotinib-sensitive versus erlotinib-resistant NSCLC cell lines. Whitebars represent EGFR-mutant tumors. (B) The EGFR mutation signature published by Choi et al. PLoS ONE 2: e1226 (2007) was used to perform hierarchical clustering of primary lung adenocarcinomas. Red bars represent EGFR-mutant tumors.

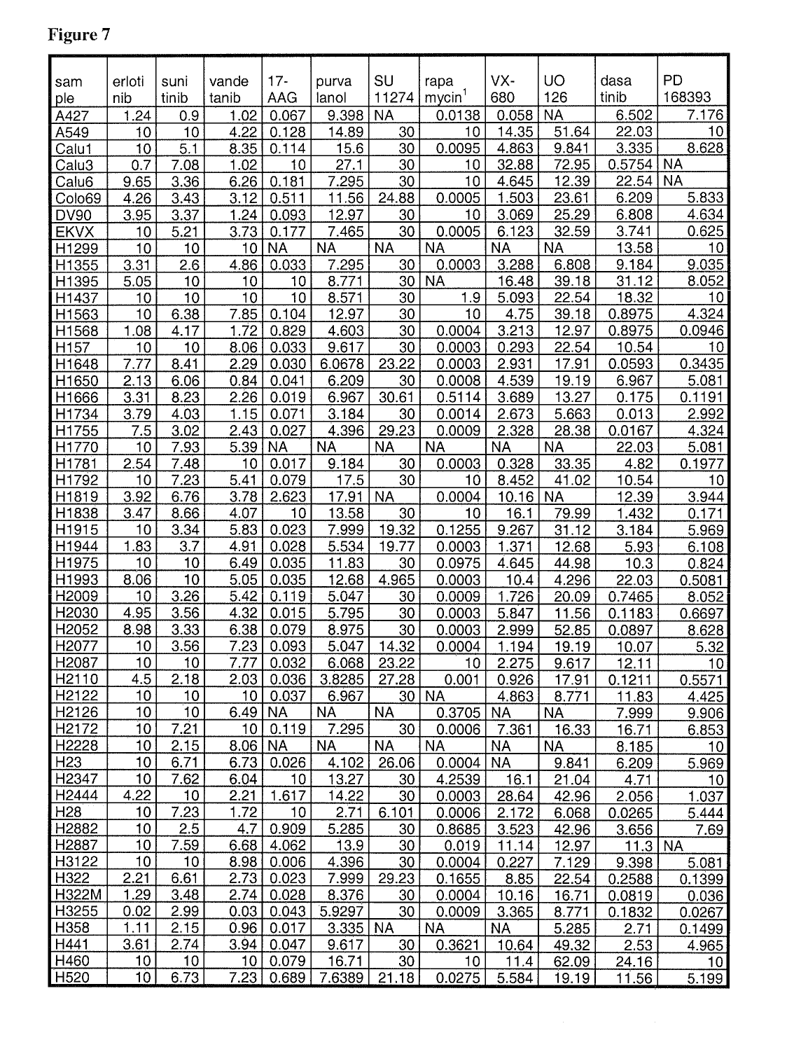

FIG. 7. Raw Sensitivity Data for Screened Compounds

Overview over the half maximal inhibitory concentrations (IC₅₀ values; [μM]) derived form high-throughput cell line based screening and analysis of the respective preimage under the kill curve for each cell line and each compound.

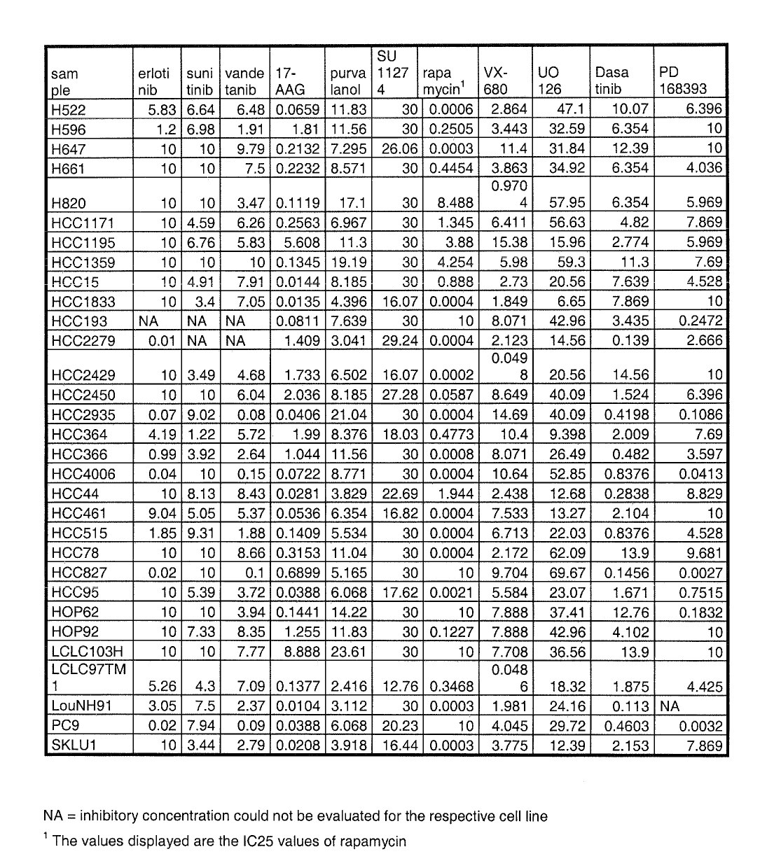

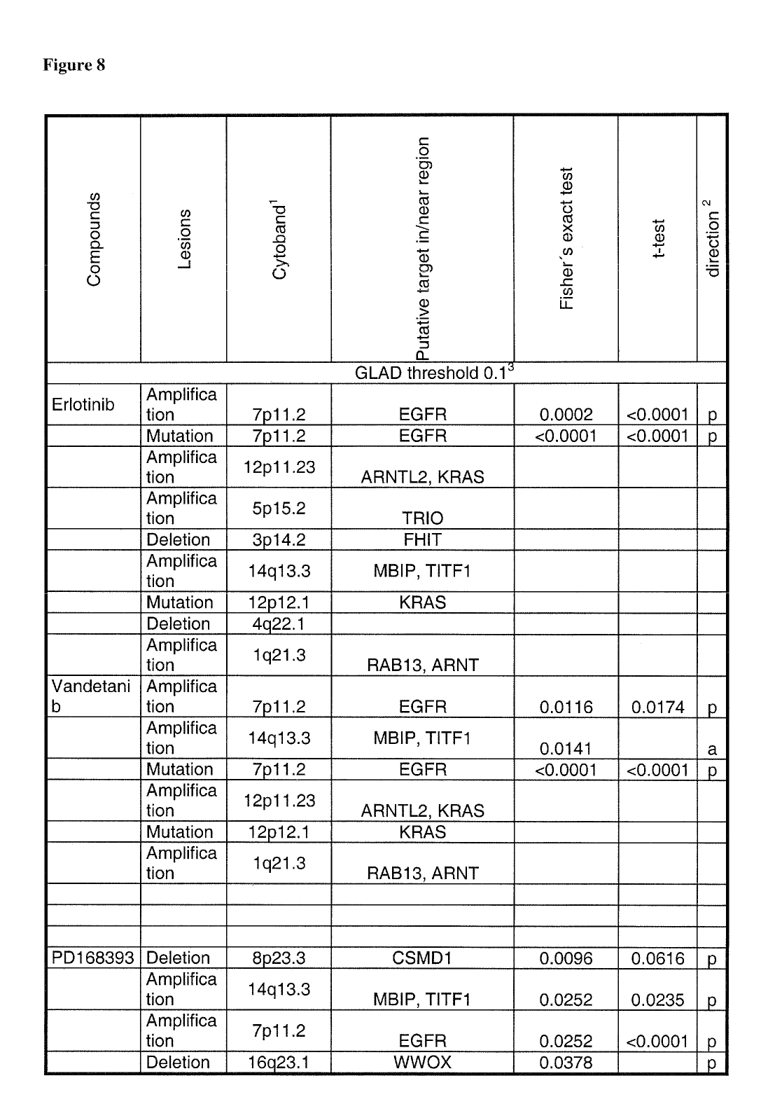

FIG. 8. Multi-Lesion Predictors of Sensitivity

Multi-lesion predictors of sensitivity tested with the KNN method. Fisher's exact test and t-test are displayed. Only significant predictors are displayed for two different GLAD thresholds.

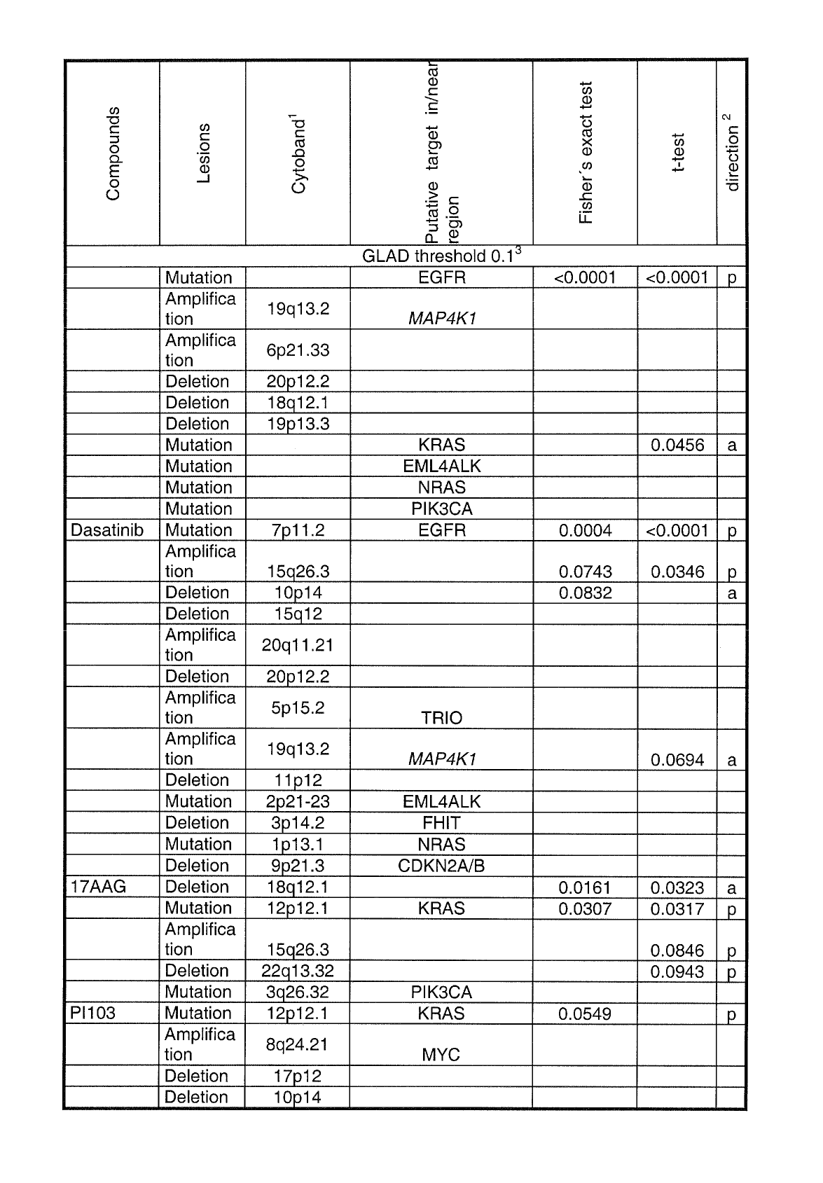

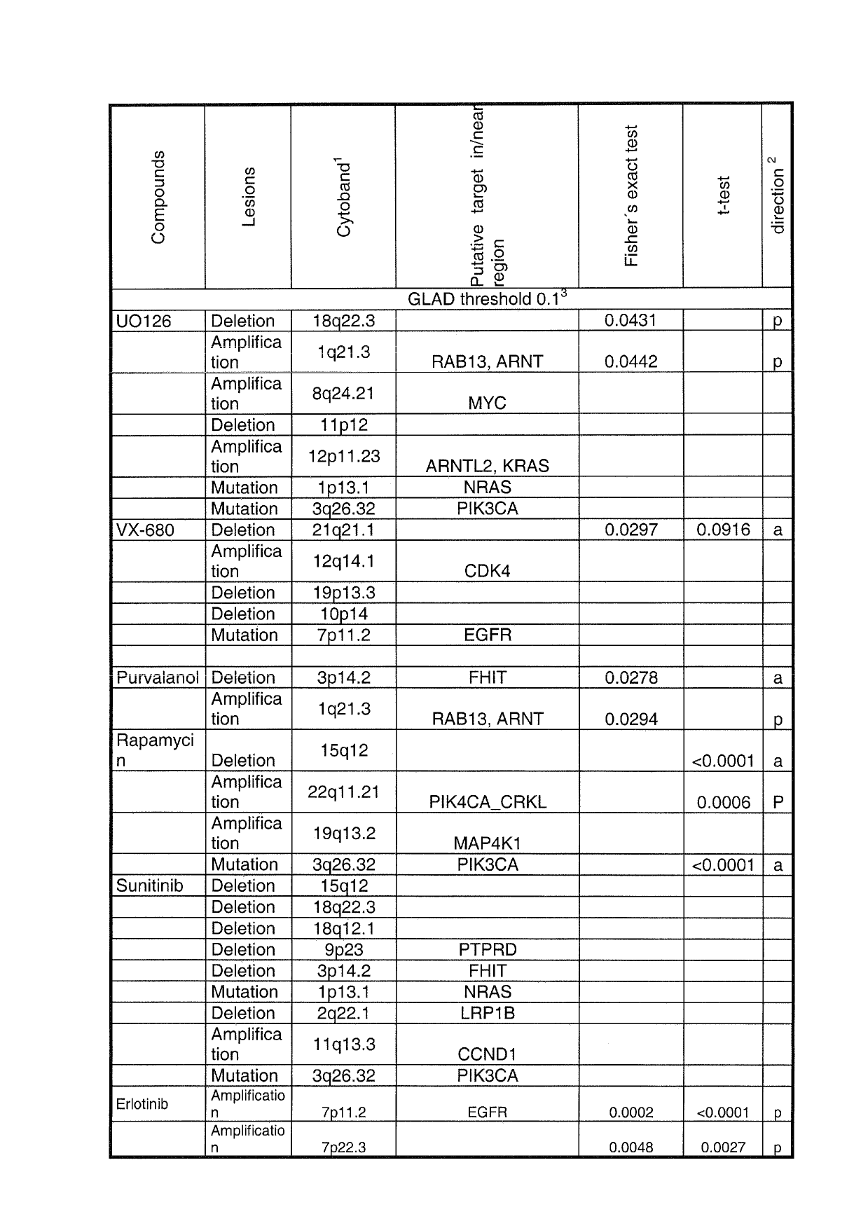

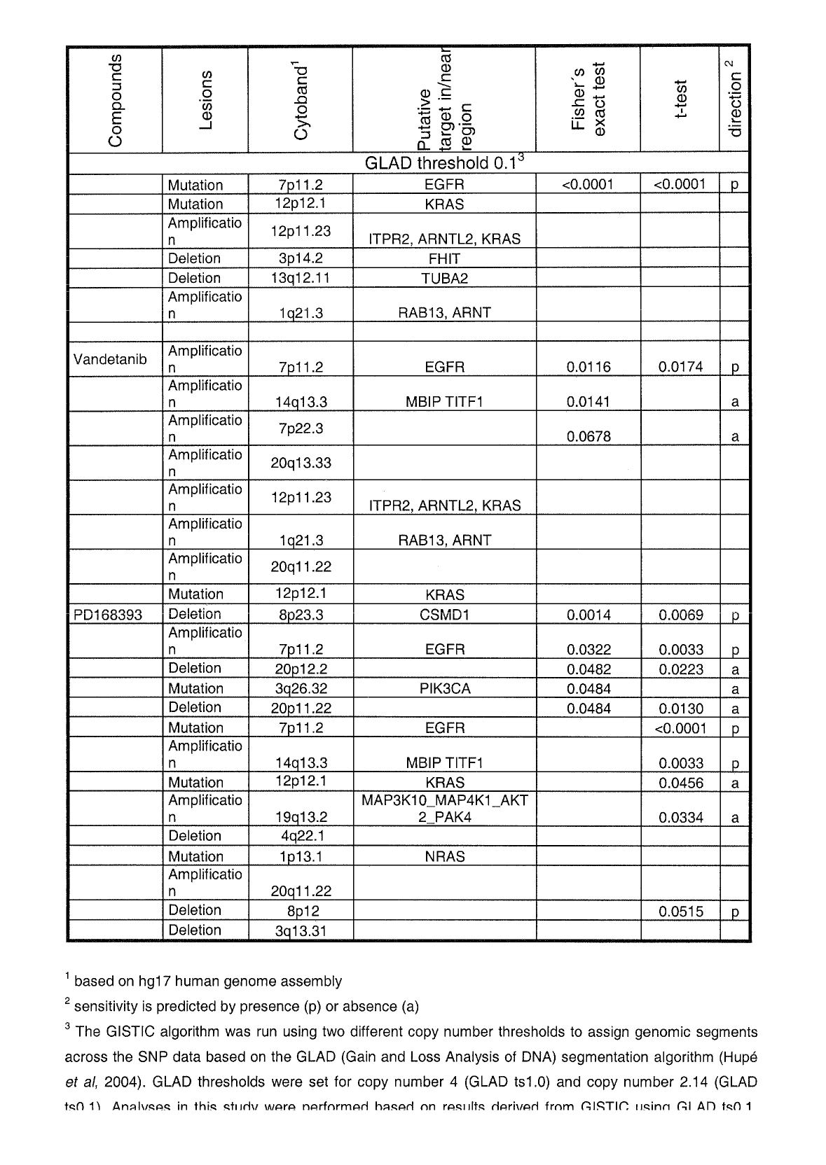

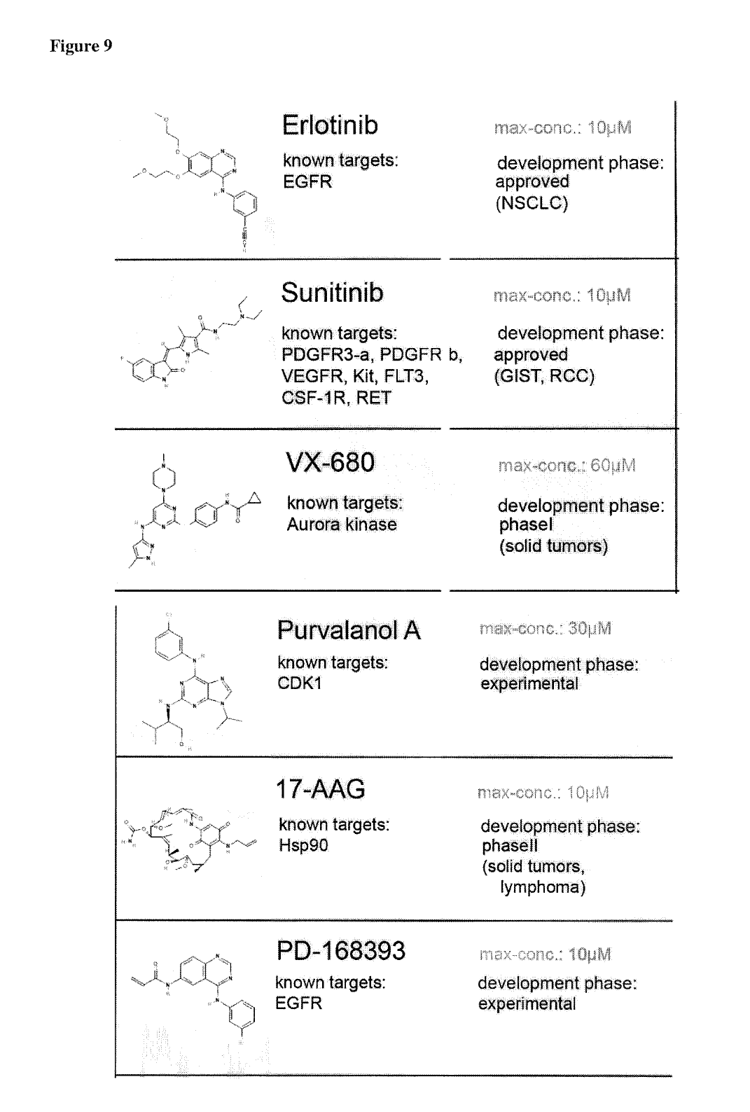

FIG. 9. Characterization of Compounds Used in the Screen

Displayed are the chemical structures, the development phase and the main known targets for all compounds used in the screening experiments

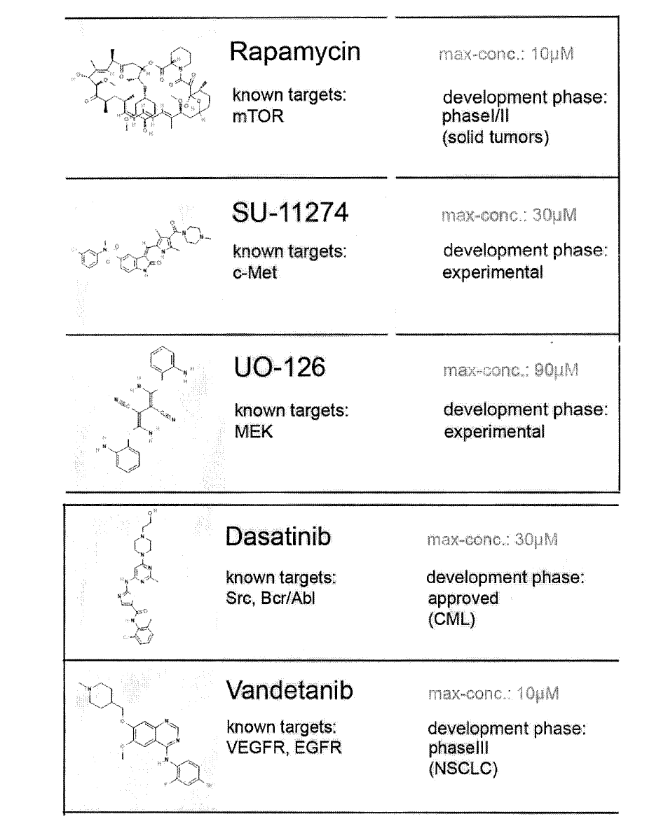

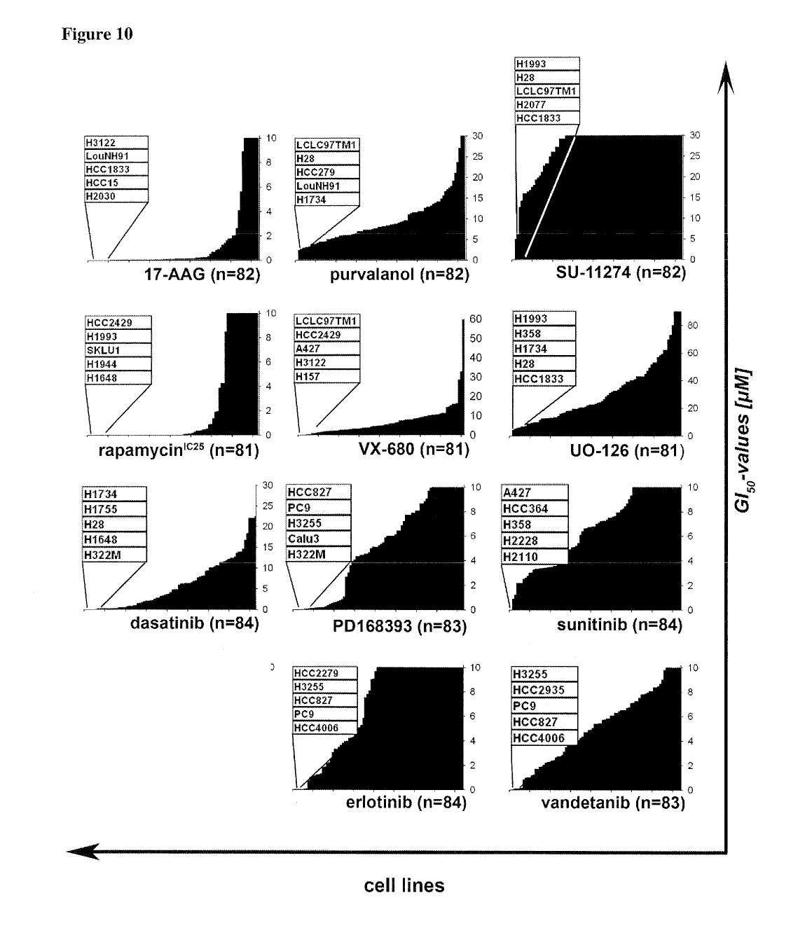

FIG. 10. Sensitivity Profiles of Compounds Determined by High-Throughput Cell Line Screening

The half-maximal inhibitory concentrations (y-axis; IC₅₀ values) for 11 compounds are shown for the entire collection of NSCLC cell lines (individual cell lines, x-axis). Due to the fact that rapamycin typically fails to completely abrogate cellular proliferation (O'Reilly et al., 2006), the 25%-inhibitory concentration is shown for this compound. Bars represent IC₅₀ respectively IC₂₅ values (y-axis) throughout the cell line collection (x-axis) ranked according to sensitivity. The maximum concentration is assigned to the IC₅₀ resp. IC₂₅ value (10 μM for 17-AAG, erlotinib, vandetanib, sunitinib and PD168393, 30 μM for SU-11274 and dasatinib, 60 μM for VX-680, 90 μM for purvalanol and UO126) for resistant cell lines.

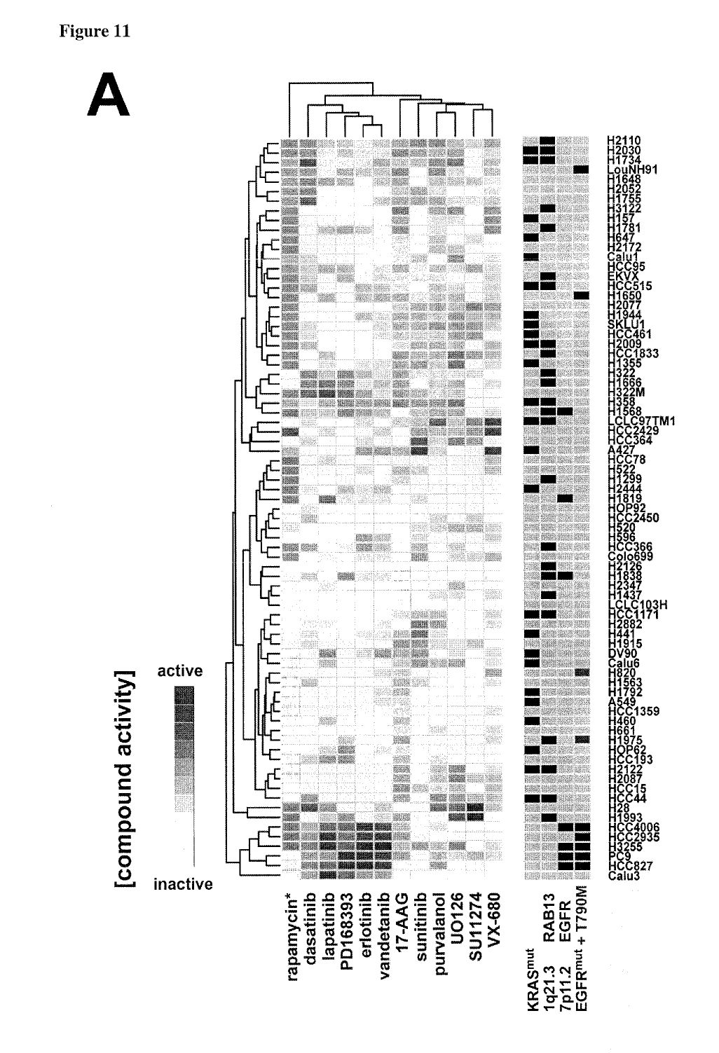

FIG. 11. Hierarchical Clustering of Compound Activity Uncovers Mutated EGFR as a Target for Dasatinib Activity.

(A) Displayed are the IC₅₀ values (red—high compound activity; white—low compound activity) after logarithmic transformation and normalization with the mean of the respective profile for all compounds (x-axis) and all cell lines (y-axis). The presence (black dot) of absence (grey dot) of relevant lesions is annotated in the right panel. EGFR inhibitors form a distinct subcluster, where EGFR-mutated samples show the highest degree of sensitivity. (B) Correlation coefficients (r²; red—high correlation; white—low correlation) of the IC₅₀ values after logarithmic transformation versus chromosomal gain (amp) and loss (del) as well as oncogene mutations (mut); copy numbers changes in regions defined by GISTIC were dichotomized and merged together with the binary mutation data. Putative target genes inside (#) and bordering (*) the region defined by GISTIC are annotated. Again, EGFR inhibitors are grouped in a separate cluster. (C) upper panel: Crystallographically determined binding mode of erlotinib (grey sticks) to wild-type EGFR. Dasatinib represented as white to dark grey ballandsticks is modeled into the ATP binding site of EGFR. lower panel: The T790M mutation at the gatekeeper position of the ATP pocket, associated with secondary EGFR-inhibitor resistance in patients displaces both erlotinib and dasatinib from the ATP binding pocket of the kinase domain. (D) Upper panel: Ba/F3 cells expressing mutant (del Ex19 or Ex19/T790M) EGFR were treated for 12 h with the indicated concentrations of either dasatinib or erlotinib and whole-cell lysates were immunoblotted for phospho-EGFR and EGFR. Lower panel: Dose-dependent growth inhibition after 96 h treatment with either dasatinib or erlotinib was assessed measuring cellular ATP content.

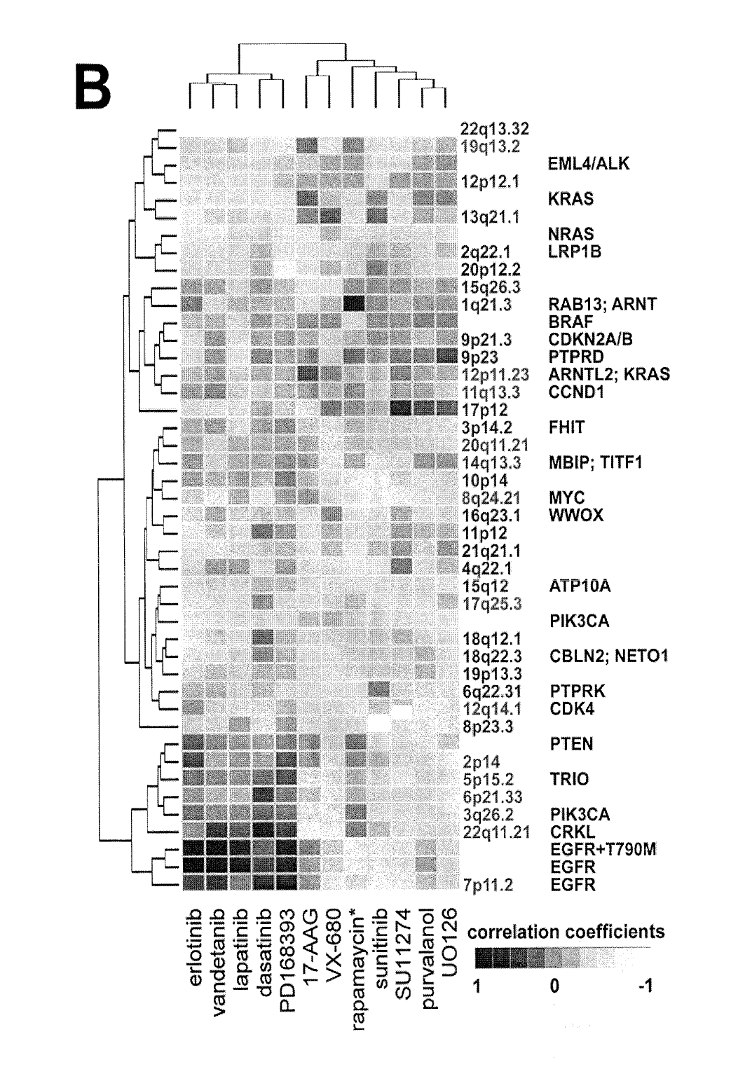

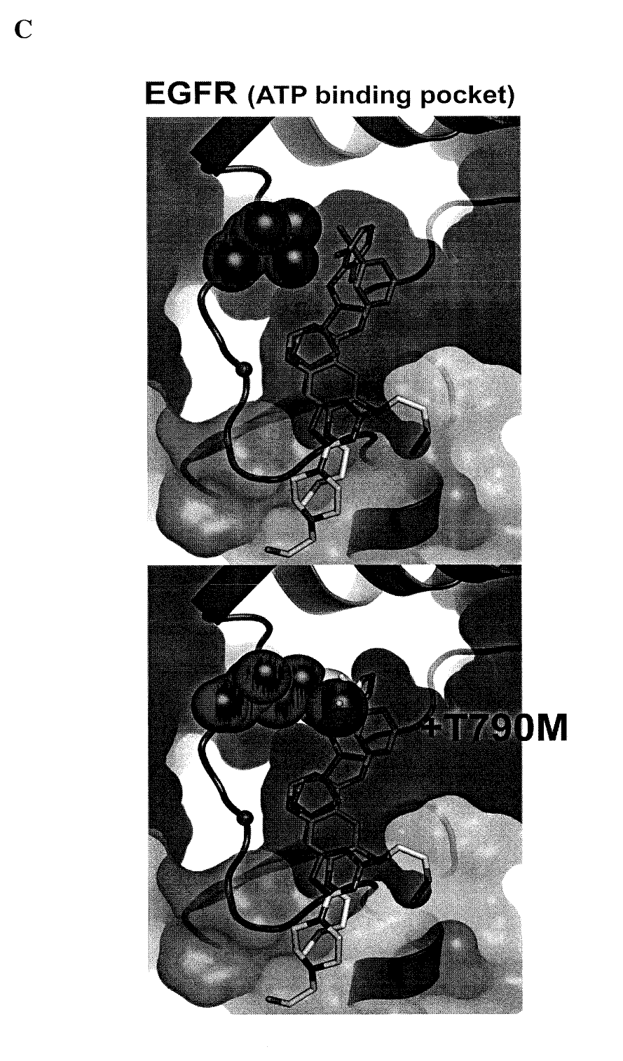

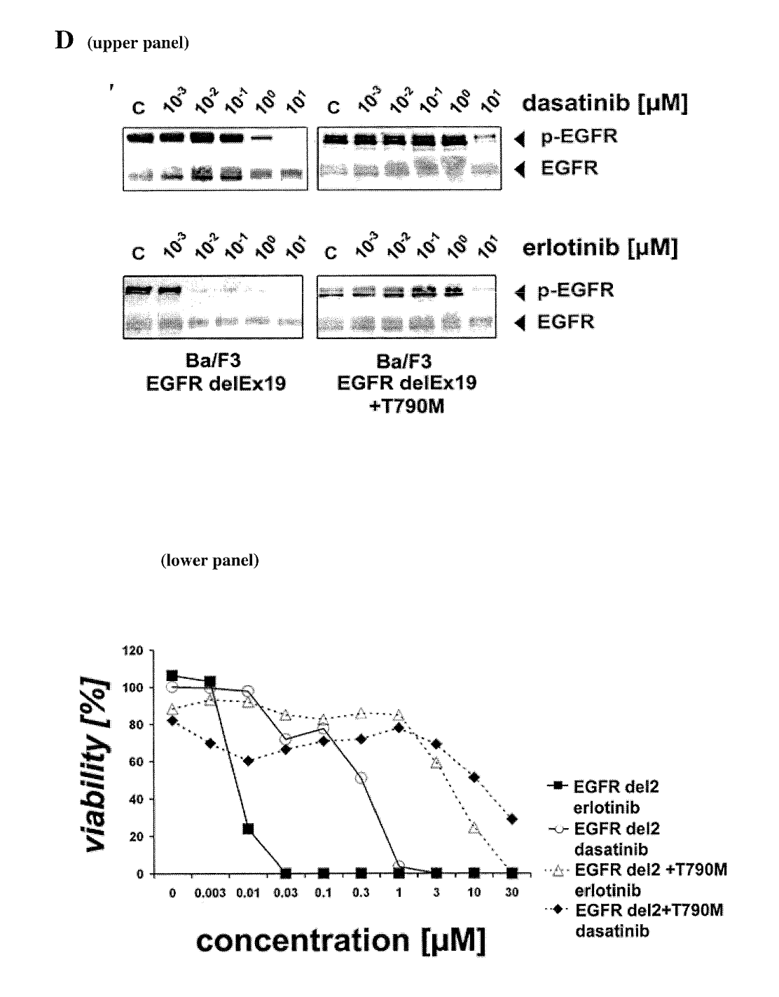

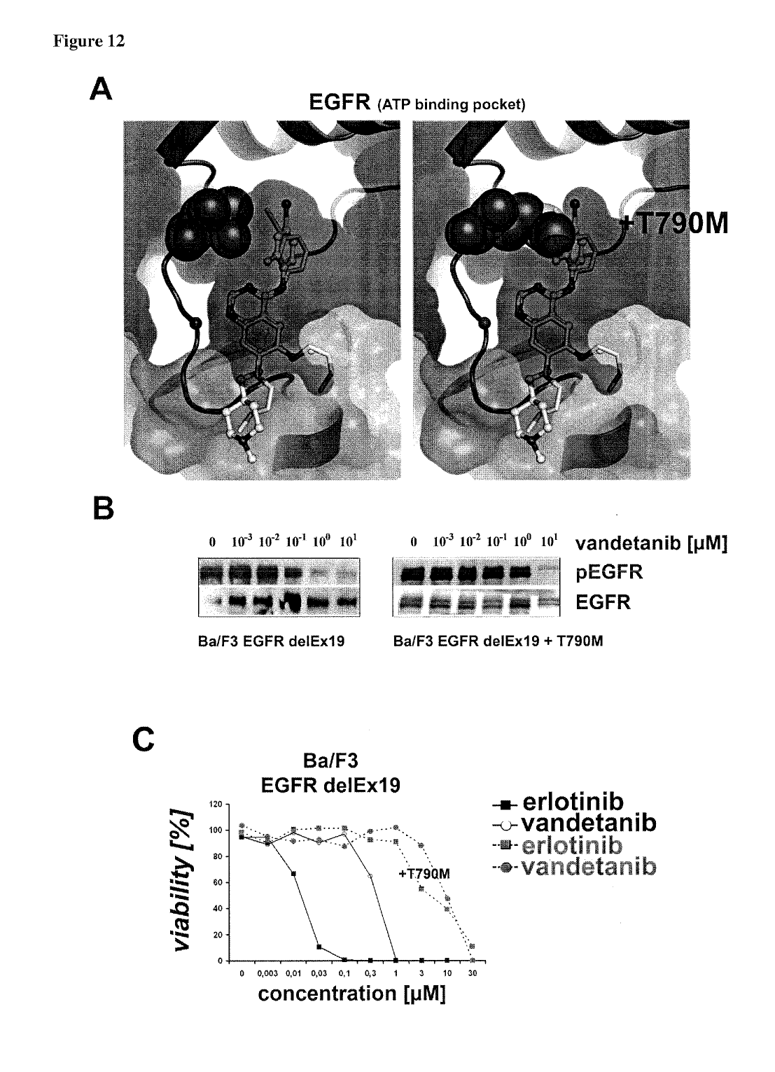

FIG. 12. Mutated EGFR as a Target for Vandetanib Activity.

(A) left panel: Vandetanib represented as ballandsticks is modeled into the ATP binding site of EGFR based on its crystallographically determined binding mode with the RET kinase. right panel: The T790M mutation at the gatekeeper position of the ATP pocket, associated with secondary EGFR-inhibitor resistance in patients displaces the drug front the ATP-binding pocket. The crystographically determined binding mode of erlotinib asballandsticks is shown in both panels as an overlay for reference. (B) Ba/F3 cells expressing mutant (del Ex1.9 or Ex191T790M) EGFR were treated for 12 h with the indicated concentrations of either vandetanib or erlotinib and whole-cell lysates were immunoblotted for phospho-EGFR and EGFR. (C) Dose-dependent growth inhibition after 96 h treatment with either dasatinib or erlotinib was assessed measuring cellular ATP content.

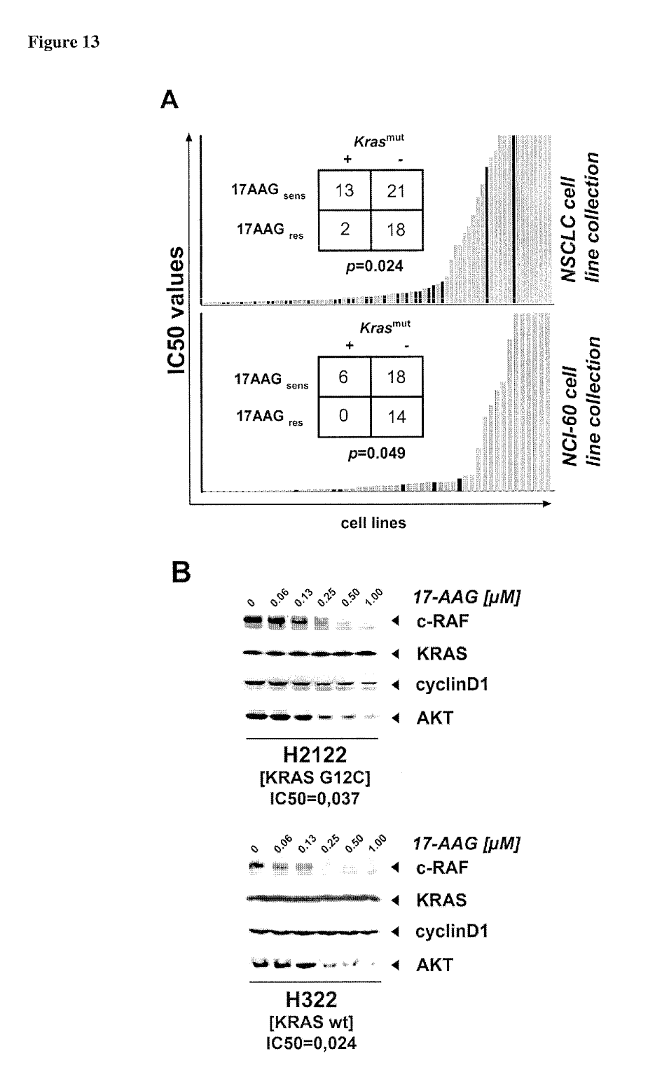

FIG. 13. Lesion-Based Prediction for Activity of 17-AAG, UO126 and Dasatinib.

(A) Distribution of KRAS mutations (black columns) across the 17-AAG-sensitivity profile (IC₅₀ values) in the NSCLC cell line collection and the NCI-60 cell line panel. Incidence of KRAS mutation and sensitivity towards 17-AAG is represented by a Fisher's exact test for both datasets. (B) Lysates of a KRAS wildtype (wt) and a KRAS mutated (G12C) cell line treated with 17AAG at different concentrations were immunoblotted for c-RAF, KRAS, cyclinD1 and Akt. (C) Distribution of copy number gain at 1q21.3 (black columns) across the UO126-sensitivity profile (IC₅₀ values) in the NSCLC cell line collection and the hypothemycin-sensitivity profile in the NCI-60 cell line panel. Incidence of amplification of 1q21.3 mutation and sensitivity towards 17-AAG is represented by a Fisher's exact test for both datasets. (D) Cell lines were sorted according to their sensitivity to dasatinib (IC50<1 μM; light grey). Strikingly, most dasatinib-sensitive NSCLC cell lines are found among those with highest copy number for members of the SRC and Ephrin receptor kinase families.

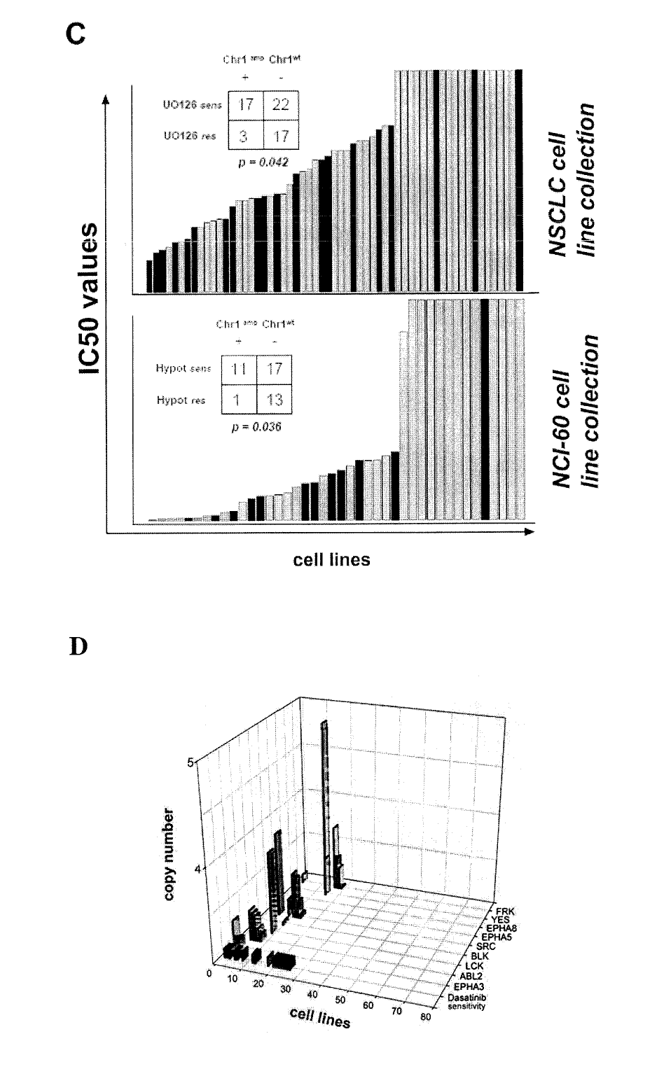

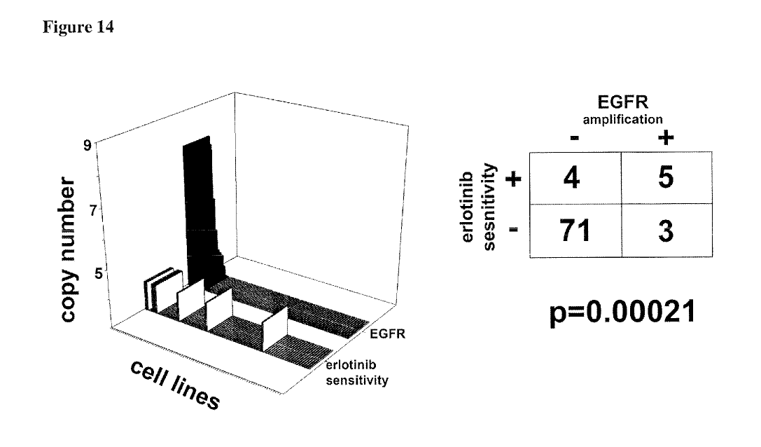

FIG. 14. Validation of the Target-Enriched Sensitivity Prediction Method that Yielded Genomic Predictors of Dasatinib Sensitivity.

All cell lines were sorted according to their sensitivity to erlotinib (IC₅₀<1 μM; greybars). Cell lines enriched for sensitivity to erlotinib are found among those with highest copy numbers for EGFR. The contingency table for EGFR (dark grey bars) amplification and erlotinib sensitivity including the p-value determined using fisher's exact test are displayed in the right panel.

FIG. 15

Cluster Image of the NSCLC Cell Lines Against Dasatinib Using the 6 Gene Signature Published by Huang et al., (2007)

To evaluate the predictive value of the 6 gene signature proposed by Huang (2007), Cancer Res 67, 2226-2238, the expression levels of the respective genes were analyzed by hierarchical clustering with the dasatinib sensitivity denoted using the annotation by 0 and 1. The samples with a similar expression profile across these genes are found in the same subcluster. Bright spots represent genes that are repressed, dark spots represent genes that are overexpressed when compared to average mRNA expression levels.

FIG. 16

Prostate/Breast Cancer-Gene Signature Associated with Dasatinib Sensitivity

To evaluate the predictive value of a gene signature proposed by Huang (2007). Cancer Res 67, 2226-2238, the expression levels of the respective genes were analyzed by hierarchical clustering with the dasatinib sensitivity denoted using the annotation by 0 and 1. The samples with a similar expression profile across these genes are found in the same subcluster. Bright spots represent genes that are repressed, dark spots represent genes that are overexpressed when compared to average mRNA expression levels.

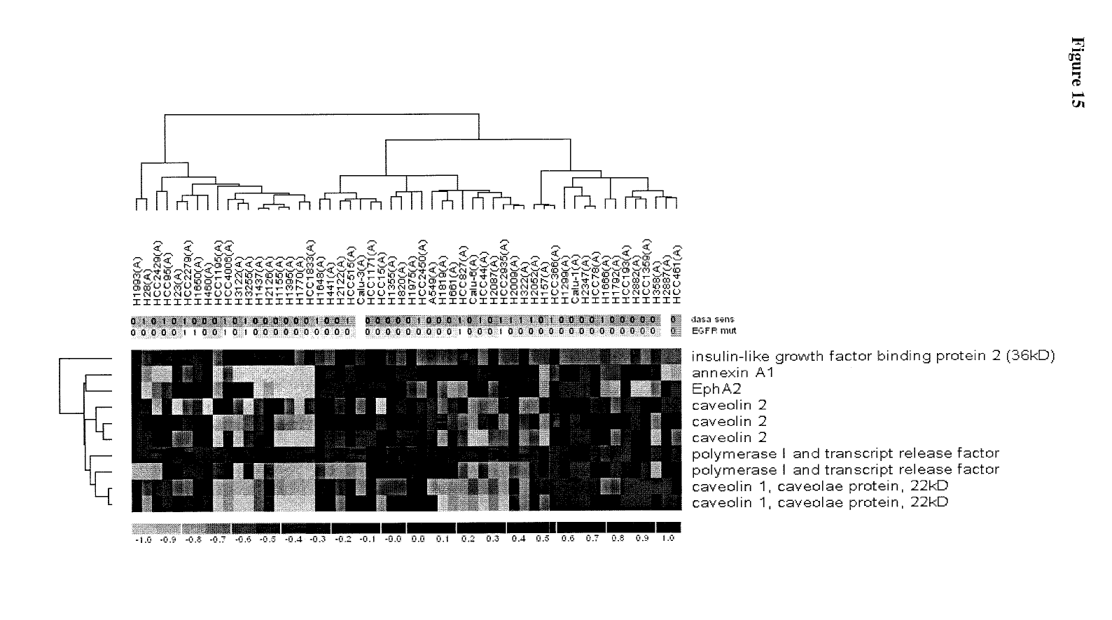

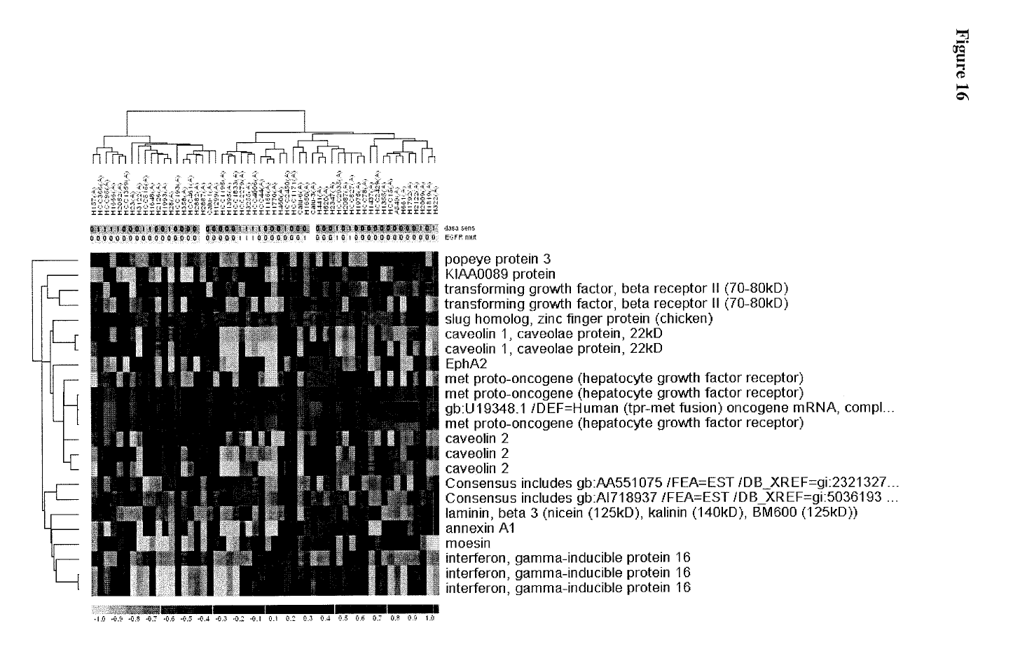

FIG. 17

All Genes Associated with Dasatinib Sensitivity in Prostate Cancer

To evaluate the whole gene set signature proposed by Huang (2007), Cancer Res 67, 2226-2238, the expression levels of the respective genes were analyzed by hierarchical clustering with the dasatinib sensitivity denoted using the annotation by 0 and 1. The samples with a similar expression profile across these genes are found in the same subcluster. Bright spots represent genes that are repressed, dark spots represent genes that are overexpressed when compared to average mRNA expression levels.

FIG. 18. NSCLC Cell Line Panel

FIG. 18 shows a list of cell lines in the initially tested NSCLC cell line panel, their respective KRAS mutations and the corresponding half-maximal inhibitory concentrations (1050).”

FIG. 19. Cell Lines

FIG. 19 shows a set of cell lines which are to be used in accordance with the screening methods provided herein for the identification of drugs, which can be used in anti-cancer treatment/anti-proliferative treatment.

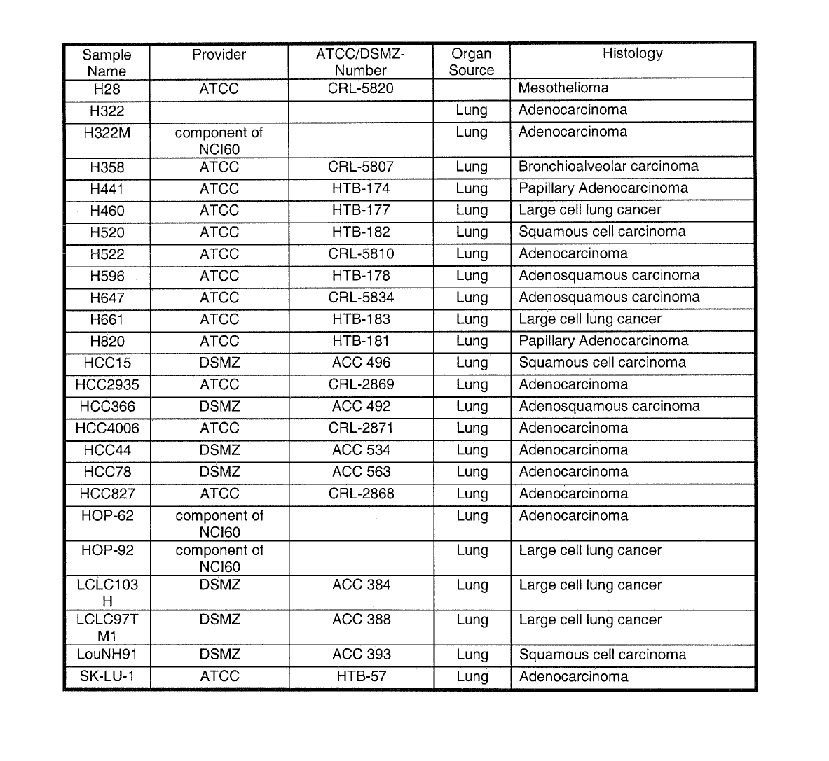

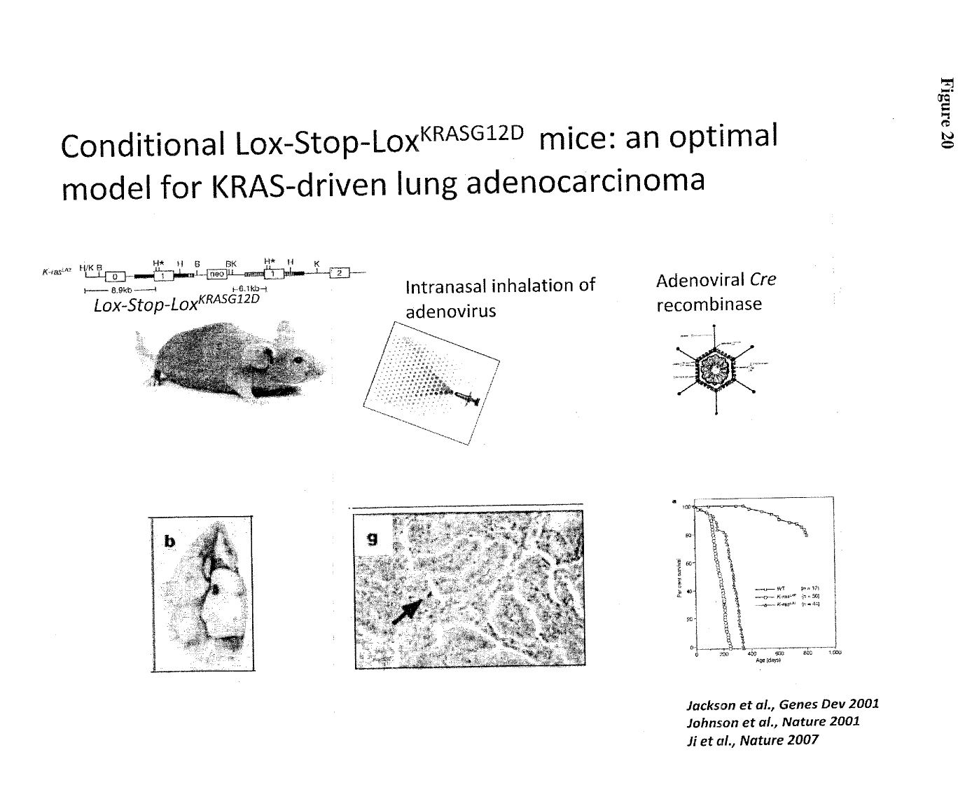

FIG. 20. A Transgenic Mouse Model of KRAS-Mutant Lung Cancer Transgenic mice expressing a G12D mutation of KRAS specifically in the lung were generated by intranasal application of adenoviral Cre recombinase to Lox-Stop-Lox^{KRASG12D} mice (upper panels from left to right) as described in the literature (Jackson, E. L. et al., Genes Derr 15:3243-3248 (2001); Johnson L. et al., Nature, 410(6832):1111-6 (2001) and Ji H. et al., Nature. 448(7155):807-10 (2007)). These mice develop lethal lung adenocarcinomas at high penetrance (lower panels from left to right). Note that the images were taken from the earlier publications for illustration purposes only.

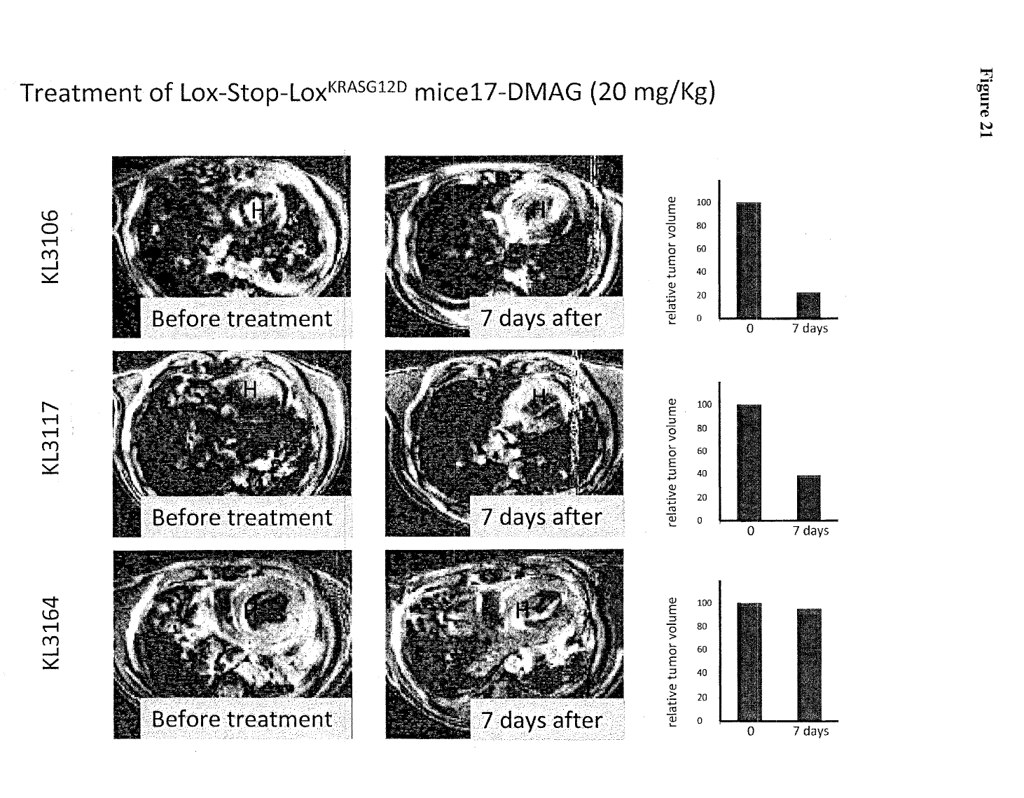

FIG. 21. Treatment of Lox-Stop-Lox^{KRASG12D} Mice with HSP90 Inhibitor

Transgenic mice (as described in FIG. 20) with KRAS-mutant lung tumors were treated with the HSP90 inhibitor 17-DMAG for seven days. Tumor volumes were determined by magnetic resonance imaging and shown as transthoracic images (left panels), quantified and changes in tumor volume relative to the pre-therapy images are given in percent.

The Examples illustrate the invention.

### EXAMPLE 1

### Methods and Results

### Cells

Cells were obtained from ATCC (www.atcc.org), DSMZ (www.dsmz.de), from own or from other cell culture collections. Details on all cell lines are listed in FIG. 1. This also contains information on providers and on culture conditions. Cells were routinely controlled for infection with mycoplasm by MycoAlert (www.camhrex.com) and were treated with antibiotics according to a previously published protocol (Uphoff and Drexler. 2005) in case of infection.

### SNP Arrays

Genomic DNA was extracted from cell lines using the PureGene kit (www.gentra.com) and hybridized to high-density oligonucleotide arrays (Affymetrix, Santa Clara, Calif.) interrogating 238,000 SNP loci on all chromosomes except Y, with a median intermarker distance of 5.2 kb (mean 12.2 kb; www.affymetrix.com). Array experiments were performed according to manufacturer's instructions. SNPs were genotyped by the Affymetrix Genotyping Tools Version 2.0 software. SNP array data of primary samples were obtained from the Tumor Sequencing Project (http://www.genome.gov/cancersequencing/). We applied a novel and general method for Genomic Identification of Significant Targets in Cancer (GISTIC) (Beroukhim et al., 2007) to analyze the dataset. Each genomic marker is scored according to an integrated measure of the prevalence and amplitude of copy-number changes (and only prevalence in the case of LOH), and the statistical significance of each score is assessed by comparison to the results expected from the background aberration rate alone. The GISTIC algorithm was run using two different copy number thresholds to assign genomic segments across the SNP data using the Gain and Loss Analysis of DNA (GLAD) segmentation algorithm (Rupe et al., 2004) with copy number 4 (GLAD threshold 1.0) and copy number 2.14 (GLAD threshold 0.1).

### Detection of Homozygous Deletions

For the identification of homozygous deletions, SNP data were filtered for five coherent SNPs exhibiting a copy number loss of <0.5. The analysis was focused on focal losses, excluding entire chromosomal arms. Information about genes located in a region of homozygous deletion was based on hg17 build of the human genome sequence from the University of California, Santa Cruz (http://genome.ucsc.edu).

### Analysis of Co-Occurring Lesions

The analysis was performed computing ratios of observed vs. expected co-occurrence frequency of individual lesions. Hierarchical clustering of mutation data combined to a dichotomized version of quantitative copy number changes was performed using the reciprocal co-occurrence ratio as distance measure with average linkage method. As the adequate threshold for occurrence of copy number lesions depends on the overall level of copy number alteration for that specific lesion, the sum of these ratios for three distinct thresholds was used.

### Mutation Detection

Mutation status of known oncogene mutations in the genes EGFR, BRAF, ERBB2, PIK3CA, NRAS, KRAS, ABL1, AKT2, CDK4, FGFR1, FGFR3, FLT3, JAK2, KIT, PDGFRA and RET was determined by mass-spectrometric genotyping (Thomas et al., 2007). The mutation status of these genes for all cell lines was published previously (Thomas et al., 2007). In addition, the genes EGFR, BRAF, ERBB2, PIK3CA, KRAS, TP53, STK11, PTEN and CDKN2A were bidirectionally sequenced following PCR-amplification of all coding exons.

Mutation detection for choice of appropriate therapy depending on the respective mutation has been further developed to compensate for the methodological issues connected with sequencing of tumor samples with high admixture of non-tumoral cells. In our laboratory we have therefore developed the following algorithm: if the tumor content of the tumor specimens is higher or equal than 70% estimated by conventional histomorphology, we have found Sanger dideoxy-chain-termination sequencing to be optimal in terms of cost-efficiency and sensitivity. However, when the tumor content is between 70% and 20% we have found conventional pyrosequencing as, for example, implemented in the Biotage instrument, to deliver higher sensitivity and specificity at acceptable costs. If the tumor content is lower than 20%, we have found massively parallel next-generation sequencing, as for example implemented in the Roche-454 sequencing system (Thomas et al., Nature Medicine July; 12(7):852-5 2006), to be the most sensitive and accurate method in this setting. Together, this algorithm provides high sensitivity in all settings combined with maximum cost-efficiency.

### Expression Arrays

Expression data were obtained using Affymetrix. U133A arrays from 54 of the cell lines. RNA extraction, hybridization and scanning of arrays were performed using standard procedures (Bhattacharjee et al., 2001). CEL files from U133A arrays were preprocessed using the dChip software. We compared the cell lines to primary lung cancer, renal cell carcinomas and lymphoma specimens as well as to the respective cell lines by hierarchical clustering. For comparison with expression profiles from further entities, we used lung cancer (Lu et al., 2006), renal cell carcinoma (Lenburg et al., 2003) (Shankavaram et al., 2007) and lymphoma (Hummel et al., 2006; Rinaldi et al., 2006) specimen datasets as published in GEO array (http://www.ncbi.nlm.nih.gov/geo/). Data were processed by standard procedures, normalization was performed in dChip (http://biosunl.harvard.edu/complab/dchip/). For comparison of NSCLC cell lines (U133A) and primary tumors, we used data on adenocarcinomas from Bhattacharjee and colleagues generated on U95Av2 arrays. Genes differentially expressed between cell lines with mutated EGFR and wild type EGFR (fold change between groups >2 [90% CI], absolute difference >100 and p<0.005) respectively between erlotinib sensitive and resistant cell lines (erlotinib sensitive (IC₅₀<0.5 μM) vs. erlotinib resistant (IC₅₀>2 μM), fold change >1.5 [90% CI], absolute difference >100, p<0.005) were selected. For principal component analysis, the R language for statistical computing was used. Variable transcripts were identified using the following filtering criteria: coefficient of variation in [1.9; 10], 40% present call rate. The first principal component described 14.5% of the overall variance, the second 9.6% and the third 8.2%. Using a cut-off of 1400, samples were grouped according to the first principal component.

### Cell-Based Screening

Erlotinib, vandetanib and sunitinib were purchased from commercial suppliers, dissolved in DMSO and stored according to manufacturer's instructions. Cells were plated into sterile microliter plates using a Multidrop instrument (www.thermo.com) and cultured overnight. Compounds were then added in serial dilutions. Cellular viability was determined after 96 h by measuring cellular ATP content using the CellTiter-Glo assay (www.promega.com.). Plates were measured on a Mithras LB940 plate reader (www.bertholdtech.com). Half-maximal inhibitory concentrations were determined from the respective preim.age under the kill curve, where the latter was smoothed according to the logistic function with the parameters appropriately chosen.

### Lesion-Based Prediction of Compound Sensitivity

For lesion-based prediction of sensitivity, three different approaches were applied. First, the most sensitive and most resistant samples were chosen according to their sensitivity profile. Where the sensitivity profile of the corresponding compound did not allow a clear distinction between resistant and sensitive cell lines, groups were defined by the 25^{th} and 75^{th} percentiles. We used. Fisher's exact test to evaluate the association between the activity of the compound and the presence of significant lesions as defined by GISTIC. For this, the cell line panel was divided according to the presence of each lesion. The logarithmically transformed IC₅₀ values pertinent to each group were now compared by a two-sample t-test. In order to avoid an artificially low variance the t-tests were based on a fixed variance determined as the mean of the variances that were clearly distinct from zero (>0.1).

In a next step, multi-lesion predictors of sensitivity were calculated using a KNN algorithm with a leave-one-out strategy (Golub et al., 1999), where the same choice of samples was used as above for Fisher's exact test: For all but one sample, genetic lesions strongly discriminating between sensitive and resistant cell lines were selected and the KNN algorithm was based on these. The prediction was validated by the remaining left-out sample. The collection of features where this validation had best performance was taken as the best combined predictor to the respective compound.

For identification of the best erlotinib single gene predictor, we dichotomized (threshold=0.7) our lesion data. Cell lines with an IC₅₀<0.07 μM were defined as sensitive. For the predictor, the same cutoff values were used. Best performance in the leave-one-cross validation was obtained using 15 features, k=3 neighbours and the cosine-based metric. Due to the problem of multiple hypotheses testing, the significance of the above t-tests as well as Fisher's exact tests should be understood in an explorative rather than confirmative sense.

In order to validate the finding that single lesions may be associated with sensitivity to a specific inhibitor, we made use of the NCI-60 cancer cell line panel (http://dtp.nci.nih.gov/mtargets/mt_index.html). Since the MEK inhibitor UO126 and the Hsp90 inhibitor 17-AAG were not covered by the collection of pharmacological data, we analyzed the association of the respective lesions to hypothemycin (MEK inhibitor) and to geldanamycin (17-AAG is a geldanamycin derivate) instead. In order to compare the association between lesions and sensitivity, the number of cell lines classified as sensitive or resistant in the lung cancer cell line panel was adapted for the NCI-60 cell line collection for the respective compounds. Significance of association was analyzed by a Fisher's exact test. Due to discordant IC₅₀ values the cell lines H0P62 and A549 were excluded from the analysis with respect to the Hsp90-inhibitors. The thresholds for 1q21.3 amplification were set according to the overall distribution of copy number changes in the respective dataset (2.7 corresponding to 33% of the NSCLC cell lines; 2.4 corresponding to 33% of the NCI-60 collection).

To evaluate the 6 gene signature proposed by Huang et al. (2007), the expression levels of the respective genes were analyzed by hierarchical clustering (FIG. 17) with the dasatinib sensitivity denoted using the annotation by 0 and 1. The samples with a similar expression profile across these genes are found in the same subcluster. However, the dasatinib-sensitive samples did not form a specific subcluster but were distributed randomly in the columns, suggesting that the signature under consideration is not suitable for prediction of dasatinib vulnerability.

### Generation of EGFR-Mutant Ba/F3 Cells

EGFR_cDNA was subcloned into pBabe-hygro vectors. The most prevalent NSCLC-derived mutants (http://www.sanger.ac.uk/genetics/CGP/cosmic/) were introduced into the retroviral construct using site-directed mutagenesis (Quick-Change Mutagenesis XL kit: Stratagene, La Jolla, Calif., USA) and virus was packed and produced as previously described (Greulich et al., 2005). Murine Ba/F3 cells were stably transduced with the retroviruses and after IL-3 withdrawal, independently growing cells were chosen for further experiments.

### Structural Modeling of Compound Binding

The crystal structure of dasatinib and vandetanib bound to the RET kinase (pdb code 21VU (Knowles et al., 2006)) was aligned to the kinase domain of EGFR bound to erlotinib (pdb code 1M17 (Stamos et al., 2002)) using PyMol (http://www.pymol.org). Based on the structural alignment of Abl with EGFR, the binding mode for dasatinib in EGFR is identical to the dasatinib-Abl complex. The piperazine moiety of the inhibitor points out of the ATP site into the solvent while the 2-amino-thiazole forms two hydrogen bonds with the hinge region of the kinase (N3 of the thiazole ring with the amide nitrogen of Met793 Met318 in Abl) and the 2-amino hydrogen of dasatinib with 0 of Met793 (Met318 in Abl). An additional hydrogen bond can form between the side chain hydroxyl of the gatekeeper Thr790 (Thr315 in Abl) and the amide nitrogen of the inhibitor. The chloro-methyl-phenyl ring of dasatinib binds into a hydrophobic pocket near the gatekeeper Thr790 and helix C and would clearly clash with the Met side chain of drug resistant EGFR-T790M. I vandetanib, N1 of the quinazoline scaffold forms one key hydrogen bond to the backbone of the hinge region (Met793 in EGFR, Ala807 in RET kinase). The bromo-fluoro-phenylamine moiety of vandetanib adopts a conformation similar to the ethynyl-phenylamine of erlotinib being close to the side chain of Thr790 in EGFR and Val804 in RET kinase. Figures of the structures were prepared using PyMol.

### Western Blot Analysis

Whole-cell lysates were prepared in NP40 lysis buffer (50 mmol/L Tris-HCl (pH 7.4). 150 mmol/L NaCl, 1% NP40) supplemented with protease and phosphatase inhibitor I and II cocktails (www.merckbiosciences.co.uk/g.asp?f=CBC/home.html) and clarified by centrifugation. Protein concentrations were determined using the Bicinchoninic Acid Protein Assay kit (www.piercenet.com) and equivalent amounts (40-60 μg) were subjected to SDS-PAGE on 12% gels, except where indicated. Western blotting was done as described previously (Shimamura et al., 2006). Anti-EGFR, anti-phospho-EGFR (Tyr¹⁰⁶⁸) and anti-pAkt antibodies were purchased from Cell Signaling Technology (Beverly. MA). Anti c-raf and anti-cyclin D1 antibodys were purchased from Santa Cruz. Anti KRAS antibody was purchased from Merck.

### Results

### A Genomically Validated Collection of NSCLC Cell Lines

84 NSCLC cell lines were collected from various sources (FIG. 1) and formed the basis for all subsequent experiments. Cell lines were derived from tumors representing all major subtypes of NSCLC tumors, including adenocarcinoma, squamous-cell carcinoma and large-cell carcinoma.

The genomic landscape of these cell lines were characterized by analyzing gene copy number alterations using high-resolution single-nucleotide polymorphism (SNP-) arrays (250K Styl; www.affymetrix.com) We used a novel analytical algorithm called Genomic Identification of Significant Targets in Cancer (GISTIC) to statistically distinguish biologically relevant lesions from background noise (Beroukhim et al., 2007). This method assigns a statistical score to each chromosomal marker reflecting both the mean amplitude and frequency of alterations at a given locus within a data set. The application of GISTIC revealed 16 regions of recurrent, high-level copy number gain (inferred copy number >2.14) and 20 regions of recurrent copy number loss (inferred copy number <1.86) (FIG. 2). Overall, we identified focal peaks with a median width of 1.45 Mb (median 13.5 genes/region) for amplifications and 0.45 Mb for deletions (median 1 gene/region). These regions contain lesions known to occur in NSCLC (e.g., deletion of LRP1B (2q). FHIT (3p). CDKN2A (9p); amplification of MYC (8q), EGFR (7p) and ERBB2 (17q); (FIG. 3A and FIG. 2). Furthermore, within broad regions of copy number gain we also identified amplification of the TITF1 (14q) and TERT (5p) (FIG. 3A and FIG. 2), recently identified by large-scale genomic profiling of primary lung adenocarcinomas (Kendall et al., 2007; Lockwood et al., 2008: Weir et al., 2007).

Analysis of homozygous deletions (HD) as well as loss of heterozygosity (LOH) is typically hampered by admixture of non-tumoral cells in primary tumors. The purity of cell line DNA permitted identification of previously unknown HD and regions of LOH, including LOH events resulting from uniparental disomy (e.g. copy-neutral events). Regions targeted by LOH encompassed genes shown to be affected by LOH in other tumor types as well as previously unrecognized LOH targets such as TSPAN5 (4q), LRDD (11p), SIRT3 (11p), NLRP6 (11p), BCL2L14 (12p), CDK8 (13q), BCL2L12 (18q), DAPK3 (19p), or UHRF1 (19p). In this analysis known genes such as MTAP (9p) and LATS2 (13q) were altered by HD (Chen et al., 2005; Schmid et al., 1998) and we found novel HD of genes such as TUBA2 or FRK. Of note, most of these regions could also be identified in primary NSCLC tumors as deleted (Weir et al., 2007); however, inferred copy numbers only inconstantly showed LOH or HD, indicating admixture of normal diploid DNA. Thus, while a recent large-scale cancer profiling study (Weir et al., 2007) has enabled insight into the genomic landscape of lung adenocarcinoma, the use of pure populations of tumor cells further afforded discovery of previously unrecognized regions of HD and LOH.

We next compared the profile of significant amplifications and deletions in this cell line collection with that of a set of 371 primary lung adenocarcinomas (Weir et al., 2007). This comparison revealed a striking similarity between the two data sets (FIG. 3A) but not between NSCLC cell lines and gliomas or melanomas (FIGS. 4A and 4B). A quantitative analysis of similarity by computing correlations of q-values confirmed the similarity of primary lung cancer and lung cancer cell line (r²=0.77) and the lack of similarity of lung cancer cell lines and primary gliomas (Beroukhim et al., 2007) (r²=0.44) or melanoma cell lines (Lin et al., 2008) (r²=0.44; FIG. 4C). Repeated random splitting of the lung cell line data and computation of internal similarity indicated correlations between 0.82 and 0.86 whereas we found no correlation with normal tissue (r²=0.0195; FIG. 4C). These results demonstrate that the genomic copy number landscape of NSCLC cell lines reflects that of primary NSCLC tumors, while tumors or cell lines of other lineages show a much lower degree of similarity (Greshock et al., 2007; Jong et al., 2007). Furthermore, the distribution of oncogene mutations in the cell lines was similar to those in primary NSCLC tumors with a high prevalence of mutations in the KRAS and EGFR genes (Aviel-Ronen et al., 2006; Bamford et al., 2004; Sharma et al., 2007; Thomas et al., 2007) and rare occurrence of PIK3CA and BRAF mutations (FIG. 3B). These results further validate our cell line collection on a genetic level.

The availability of two dimensions of genetic information (chromosomal copy number alterations and mutations) from the NSCLC cell lines enabled us to analyze the interactions of both types of lesions (FIG. 3C). Hierarchical clustering of lesions robustly grouped both mutations and amplification of EGFR in one subcluster (ratio Q of observed vs. expected co-occurrence: Q=4.38, p=0.001) while KRAS mutations consistently grouped in a distinct cluster. These findings corroborate prior observations in vivo where mutations in KRAS and EGFR are mutually exclusive while EGFR mutation and EGFR amplification frequently co-occur (Kaye, 2005; Pao et al., 2005b; Sharma et al., 2007). Moreover, these results strongly suggest that each of these mutations conditions the larger sets of genomic alterations in a specific manner.

Finally, in cluster analyses of gene expression data primary lung cancer specimens (Lu et al., 2006) and lung cancer cell lines shared one cluster (FIG. 3D) while renal cell carcinomas (Lenburg et al., 2003) and lymphomas (Hummel et al., 2006) clustered in a separate group. In summary, in-depth comparative analysis of orthogonal genomic data sets of a large panel of NSCLC cell lines and primary tumors demonstrates that these cell lines reflect the genetic and transcriptional landscape of primary NSCLC tumors.

EGFR Mutations Define Phenotypic Properties of Lung Tumors In Vitro and In Vivo

Activated oncogenes typically cause a transcriptional signature that can be used to identify tumors carrying such oncogenes (Bild et al., 2006; Lamb et al., 2003). However, we consistently failed to identify a gene expression signature specific for EGFR-mutant tumors using a annotated gene expression data set of 123 primary lung adenocarcinomas (Bhattacharjee et al., 2001) (data not shown). We therefore reasoned that the cellular purity of our cell lines might enable extracting such a signature and its translation to primary tumors. We applied principal component analyses on the variable genes and found a remarkable grouping of all EGFR mutated cell lines with a significant dissociation already in the first principal component (p=0.0005) contributing 14.5% to the overall variance (FIG. 5A). Similar results were obtained by hierarchical clustering (data not shown). Using genes differentially expressed in EGFR-mutant cell lines as a surrogate feature, all of the EGFR-mutant primary tumors were grouped in a distinct cluster when performing hierarchical clustering (FIG. 5B). This result was also recapitulated when selecting genes differentially expressed in erlotinib-sensitive cell lines (FIG. 6A). Furthermore, patients with a tumor expressing the signature of EGFR mutated cell lines had a better overall survival than those whose tumors did not, even when excluding EGFR-mutant tumors (FIG. 5C) as seen in vivo (Eberhard et al., 2005b). These data also point to the fact that expression signatures extracted in vitro can be used to identify biologically diverse tumors in vivo, a concept recently developed and validated (Nevins and Potti, 2007).

Others have recently characterized a transcriptional signature of EGFR mutant NSCLC: using a small set of cell lines (Choi et al., 2007). However, when analyzing primary lung adenocarcinomas with the signature described by Choi et al. EGFR-mutant samples were randomly distributed across the data set (FIG. 6B). This finding further highlights the importance of using large cell line collections in order to represent the overall genomic diversity of primary tumors.

In patients, tumor regression or “response” to EGFR inhibitors such as erlotinib is correlated with mutations in the EGFR gene (Lynch et al., 2004; Paez et al., 2004; Pao et al., 2004). To systematically identify genetic lesions associated with sensitivity to erlotinib we determined erlotinib sensitivity in all cell lines. Then, we analyzed the distribution of genetic lesions in sensitive compared to insensitive cell lines (FIG. 7) and further compared the mean sensitivity of cell lines with and without the respective genetic lesion. In both analyses, EGFR mutations were the best single-lesion predictor of erlotinib sensitivity (FIG. 5D, p<0.0001). Furthermore, we found a less stringent association with amplification of EGFR; however, only EGFR mutations were significant predictors of erlotinib sensitivity when adjusting for multiple hypothesis testing using either Bonferroni or FDR-based methods (data not shown).

We next used signal-to-noise based feature selection combined with the KNN algorithm (Golub et al., 1999; Reich et al., 2006) to build a multi-lesion predictor of erlotinib sensitivity. The best performing multi-lesion predictor comprised EGFR mutations, amplification of EGFR and lack of KRIS mutations (FIG. 8) which have all been implicated in determining responsiveness of NSCLC patients to EGFR inhibitors (Cappuzzo et al., 2005; Hirsch et al., 2005; Lynch et al., 2004; Paez et al., 2004; Pao et al., 2004; Pao et al., 2005b; Tsao et al., 2005). These results underscore the role of EGFR mutations in conferring sensitivity to erlotinib in a systematic computational analysis involving global genetic lesion profiles. Furthermore, these findings imply that essential transcriptional and cell biology phenotypes of the original tumors are preserved in the cell lines, a necessary requirement for application of such collections as proxies in preclinical drug target validation efforts.

### Differential Activity of Compounds in Clinical Development in NSCLC Cell Lines

Having validated the cell line collection by demonstrating their genomic and phenotypic similarity to primary NSCLC tumors, we reasoned that adding complex phenotypic data might elicit additional insights into how cancer genotypes impact cell biology phenotypes. We therefore established a high-throughput cell-line screening platform that enables systematic chemical perturbations across the entire cell line panel in a short time. In our initial pilot screening experiment we profiled all cell lines against 10 inhibitors that are either under clinical evaluation or showed high activity in preclinical models; these compounds target a wide spectrum of relevant proteins in cancer (FIG. 9). We treated all cell lines with these compounds and determined IC₅₀ values (FIG. 7). The resulting sensitivity patterns (FIG. 10) revealed that while some of the compounds exhibited a pronounced cytotoxic activity in a small subset of cell lines (e.g. erlotinib, vandetanib, VX-680), others were active in most of the cell lines with only a minority being resistant (e.g. 17-AAG). Only two cell lines (<2%) were resistant to all of the compounds (FIG. 7) suggesting that most NSCLC tumors might be amenable to targeted treatment. Overall, these observations are highly reminiscent of patient responses in clinical trials where limited subsets of patients experience partial or complete response while the majority of patients exhibit stable disease, no change or progression. Thus, high-throughput cell-line screening may help estimate the fraction of tumors that are sensitive to a novel compound in development.

### Identification of Relevant Compound Targets by Similarity Profiling

As an initial approach to identify shared targets of inhibitors, we performed hierarchical clustering based on similarity of sensitivity profiles (FIG. 11A) and based on the correlation between sensitivity and genomic lesion profiles (FIG. 11B). Erlotinib and vandetanib exhibited the highest degree of similarity pointing to mutant EGFR as the critical target of vandetanib in NSCLC tumor cells (FIGS. 11A and 11B). The high degree of correlation (r²=0.91; p<0.001;) of cell line IC₅₀ values for both compounds as well as structural modelling of vandetanib binding in the EGFR kinase domain which revealed a binding mode identical to that of erlotinib further corroborate this notion (FIG. 12A). Notably, this model predicted that binding of both compounds would be prevented by the T790M resistance mutations of EGFR; accordingly, Ba/F3 cells expressing erlotinib-sensitizing mutations of EGFR together with T790M were completely resistant to erlotinib and vandetanib (FIGS. 12A and 11D).

In addition to vandetanib, PD168393, a known irreversible EGFR inhibitor (Sos et al., 2008), and the SRC/ABL, inhibitor dasatinib (Shah et al., 2004) shared a cluster with erlotinib (FIGS. 11A and B). Molecular modeling of dasatinib binding to EGFR predicted a binding mode similar to that of erlotinib (FIG. 11C) with a steric clash of erlotinib and dasatinib with the erlotinib resistance mutation T790M (Kobayashi et al., 2005: Pao et al., 2005a; Yun et al., 2007) (FIG. 11C). We formally validated EGFR as a relevant tumor cell target of dasatinib by showing cytotoxicity as well as EGFR dephosphorylation (Song et al., 2006) elicited by this compound in Ba/F3 cells ectopically expressing mutant EGFR but not in those co-expressing the T790M resistance allele (FIG. 11D). Thus, our approach identified a relevant tumor-cell target of an FDA-approved drug using a systematic unbiased approach. Note that a trial of dasatinib in patients with acquired erlotinib resistance is currently ongoing (trial-Id: NCT00570401, http://clinicaltrials.gov/ct2/home); we predict limited activity of dasatinib in those patients in which acquired resistance is due to the T790M mutation.

### Supervised Learning to Identify Predictors for Inhibitor Responsiveness

As an alternative method for predicting inhibitor-responsiveness from global lesion data in a systematic fashion we applied supervised learning methods (Golub et al., 1999) as we had applied for erlotinib (see above). Applying this method we identified robust, genetic lesion-based predictors for activity of erlotinib (see above), vandetanib, PD168393, dasatinib, VX-680, 17-AAG and UO126 (FIG. 8) Supporting the results from our cluster analyses followed by structural modeling, mutations in EGFR were the best predictor for activity of erlotinib, PD168393, vandetanib and dasatinib (FIG. 8).

In our initial cluster analysis we found that KRAS mutations correlated with sensitivity to the Hsp90 inhibitor 17-AAG, a geldanamycin derivative (FIG. 13A). Using our KNN-based prediction approach, KRAS mutations were also predictive of 17-AAG sensitivity (p=0.0307, FIG. 13A. Validating this observation in an independent data set, we found the distribution of geldanamycin sensitivity and KRAS mutation in the NCI-60 cell line panel to be strikingly similar to the one observed in our panel (p=0.049; FIG. 13A). In sensitive cell lines, 17-AAG treatment reduced protein levels of c-RAF and Ala but not KRAS (FIG. 13B). Since both c-RAF and Akt are known Hsp90 clients (Basso et al., 2002; Grbovic et al., 2006), KRAS mutations may lead to dependency on activation of the Akt and RAF-MEK-ERK signaling pathways thus rendering the cells sensitive to Hsp90 inhibitors.

U0126 is a MEK inhibitor that also showed enhanced activity in a subset of the lung cancer cell line collection. Here, the KNN-prediction approach identified chromosomal gains of 1q21.3 affecting the genes ARNT and RAB13 were robustly associated with UO126 sensitivity (Fisher's exact test, p=0.0442; FIGS. 13C and 14A). In order to validate this finding in an independent data set, again we made use of the NCI-60 cancer cell line panel (Shoemaker, 2006) where hypothemycin was used as a MEK inhibitor (Solit et al., 2006). This cross-platform validation revealed that 1q21.3 gain predicts sensitivity to MEK inhibition in both datasets (Fisher's exact test, p=0.042 NSCLC cell lines; p=0.035 NCI-60 collection). Thus, cell line profiling coupled to systematic, lesion-based prediction of drug sensitivity led to predictors that could be validated in an independent data set.

### Compound Target Gene Enrichment to Predict Sensitivity

Amplification of target genes has been demonstrated to predict vulnerability to target-specific compounds in ERBB2 amplified breast cancer and EGFR amplified lung cancer. We therefore speculated that chromosomal copy number alterations of biochemically defined drug targets could be used for prediction of sensitivity to other tyrosine kinase inhibitors. To this end we used relevant tyrosine kinase inhibitor targets defined by the quantitative dissociation constant as determined in quantitative kinase assays (Karaman et al., 2008). As a proof of principal we tested whether copy number gain in EGFR is associated with sensitivity to erlotinib. In our systematic approach erlotinib sensitive cell lines were highly enriched for amplification (cn >3) of EGFR (p=0.000082) (FIG. 14). We next performed a limited screen (30 cell lines) of lapatinib, a specific inhibitor of EGFR and ERBB2. Again, we observed lapatinib sensitive cell lines to be significantly enriched in cell lines with amplified EGFR or ERBB2 genes (p=0.0265; data not shown).

Encouraged by these findings we set out to test our approach for compounds with a broad range of inhibited kinases such as dasatinib (Karaman et al., 2008). We ranked cell lines according to chromosomal copy number gain, at either one of the biochemically most sensitive dasatinib targets (K_{d}<1 nM). Those cell lines were significantly enriched for sensitivity to this compound (p=0.003, FIG. 13D). In particular, this predictor comprised gain at gene family members of Ephrin receptor and Src kinases suggesting that NSCLC cells harboring such lesions might be exquisitely sensitive to therapeutic inhibition of the encoded proteins. By contrast, copy number gain involving loci encoding biochemically less sensitive dasatinib targets failed to show enrichment of sensitive cell lines (data not shown). We therefore conclude that in NSCLC, copy number gain of Ephrin receptor or SRC family member genes renders the cells dependent on these kinases exposing a vulnerability to therapeutic inhibition with dasatinib.

### EXAMPLE 2

### Animal Data Show that Mice with KRAS-Driven Lung Adenocarcinomas are Susceptible to Treatment with an HSP90 Inhibitor

This in vivo experiment confirms that mice genetically engineered to develop KRAS-driven lung adenocarcinomas are susceptible to treatment with an HSP90 inhibitor.

These mice carry a Lox-Stop-Lox-KRAS_G12D gene. Upon administration of adenoviral Cre by nasal inhalation, these mice develop lung cancers with high penetrance, leading to rapid death from the disease. This mouse model therefore represents the most stringent and optimal model of KRAS-mutant human lung cancer and was therefore chosen for in vivo experiments; see FIG. 20. Mice received 20 mg/kg/d of 17-DMAG, a geldanamycin HSP90 inhibitor with almost identical structure as 17-AAG. In vitro data confirmed that the biological effects seen with 17-AAG in the cell lines were identical to those seen with 17-DMAG (data not shown).

After only one week of treatment 2 of 3 mice showed dramatic regression of tumors as measured by MRI imaging; see FIG. 21. The third mice showed a slight but insignificant reduction of tumor burden, comparable to stable disease. By contrast, untreated mice invariably show rapid tumor progression and die quickly from disease.

The present invention refers to the following nucleotide sequences:

The sequences provided herein are available in the NCBI database and can be retrieved from www.ncbi.nlm.nih.gov/sites/entrez?db=gene; Theses sequences also relate to annotated and modified sequences. The present invention also provides techniques and methods wherein homologous sequences, and also genetic allelic variants and the like of the concise sequences provided herein are used. Preferably, such “variants” are genetic variants.

### SEQ ID No. 1:

Nucleotide sequence encoding Homo sapiens v-Ki-ras2 Kirsten rat sarcoma viral oncogene homolog (KRAS), transcript variant b (isoform b), mRNA (>gi|34485723|ret|NM_{—}004985.3). The coding region ranges from nucleotide 182 to nucleotide 748.

### SEQ ID No. 2:

Amino acid sequence of Homo sapiens v-Ki-ras2 Kirsten rat sarcoma viral oncogene homolog (KRAS)

### SEQ ID No. 3:

Nucleotide sequence encoding mutated Homo sapiens v-Ki-ras2 Kirsten rat sarcoma viral oncogene homolog (KRAS), transcript variant a, mutation KRAS_G12V. The coding region ranges from nucleotide 182 to nucleotide 751.

### SEQ ID No. 4:

Amino acid sequence of mutated Homo sapiens v-Ki-ras2 Kirsten rat sarcoma viral oncogene homolog (KRAS), mutation KRAS_G12V

### SEQ ID No. 5:

Nucleotide sequence encoding mutated Homo sapiens v-Ki-ras2 Kirsten rat sarcoma viral oncogene homolog (KRAS), transcript variant a, mutation KRAS_G12S. The coding region ranges from nucleotide 182 to nucleotide 751.

### SEQ ID No. 6:

Amino acid sequence of mutated Homo sapiens v-Ki-ras2 Kirsten rat sarcoma viral oncogene homolog (KRAS), mutation KRAS_G12S

### SEQ ID No. 7:

Nucleotide sequence encoding mutated Homo sapiens v-Ki-ras2 Kirsten rat sarcoma viral oncogene homolog (KRAS), transcript variant a, mutation KRAS_G12A. The coding region ranges from nucleotide 182 to nucleotide 751.

### SEQ ID No. 8:

Amino acid sequence of mutated Homo sapiens v-Ki-ras2 Kirsten rat sarcoma viral oncogene homolog (KRAS), mutation KRAS_G12A

### SEQ ID No. 9:

Nucleotide sequence encoding mutated Homo sapiens v-Ki-ras2 Kirsten rat sarcoma viral oncogene homolog (KRAS), transcript variant a, mutation KRAS_G12D. The coding region ranges from nucleotide 182 to nucleotide 751.

### SEQ ID No. 10:

Amino acid sequence of mutated Homo sapiens v-Ki-ras2 Kirsten rat sarcoma viral oncogene homolog (KRAS), mutation KRAS_G12D.

### SEQ ID No. 11:

Nucleotide sequence encoding mutated Homo sapiens v-Ki-ras2 Kirsten rat sarcoma viral oncogene homolog (KRAS), transcript variant a, mutation KRAS_G12C. The coding region ranges from nucleotide 182 to nucleotide 751.

### SEQ ID No. 12:

Amino acid sequence of mutated Homo sapiens v-Ki-ras2 Kirsten rat sarcoma viral oncogene homolog (KRAS), mutation KRAS_G12C

### SEQ ID No. 13:

Nucleotide sequence encoding mutated Homo sapiens v-Ki-ras2 Kirsten rat sarcoma viral oncogene homolog (KRAS), transcript variant a, mutation KRAS_G12R. The coding region ranges from nucleotide 182 to nucleotide 751.

### SEQ ID No. 14:

Amino acid sequence of mutated Homo sapiens v-Ki-ras2 Kirsten rat sarcoma viral oncogene homolog (KRAS), mutation KRAS_G12R

### SEQ ID No. 15:

Nucleotide sequence encoding mutated Homo sapiens v-Ki-ras2 Kirsten rat sarcoma viral oncogene homolog (KRAS), transcript variant a, mutation KRAS_G12F. The coding region ranges from nucleotide 182 to nucleotide 751.

### SEQ ID No. 16:

Amino acid sequence of mutated Homo sapiens v-Ki-ras2 Kirsten rat sarcoma viral oncogene homolog (KRAS), mutation KRAS_G12F

### SEQ ID No. 17:

Nucleotide sequence encoding mutated Homo sapiens v-Ki-ras2 Kirsten rat sarcoma viral oncogene homolog (KRAS), transcript variant a, mutation KRAS_G13C. The coding region ranges from nucleotide 182 to nucleotide 751.

### SEQ ID No. 18:

Amino acid sequence of mutated Homo sapiens v-Ki-ras2 Kirsten rat sarcoma viral oncogene homolog (KRAS), mutation KRAS_G13C

### SEQ ID No. 19:

Nucleotide sequence encoding mutated Homo sapiens v-Ki-ras2 Kirsten rat sarcoma viral oncogene homolog (KRAS), transcript variant a, mutation KRAS_G13D. The coding region ranges from nucleotide 182 to nucleotide 751.

### SEQ ID No. 20:

Amino acid sequence of mutated Homo sapiens v-Ki-ras2 Kirsten rat sarcoma viral oncogene homolog (KRAS), mutation KRAS_G13D

### SEQ ID No. 21:

Nucleotide sequence encoding mutated Homo sapiens v-Ki-ras2 Kirsten rat sarcoma viral oncogene homolog (KRAS), transcript variant a, mutation KRAS_G13S. The coding region ranges from nucleotide 182 to nucleotide 751.

### SEQ ID No. 22:

Amino acid sequence of mutated Homo sapiens v-Ki-ras2 Kirsten rat sarcoma viral oncogene homolog (KRAS), mutation KRAS_G13S

### SEQ ID No. 23:

Nucleotide sequence encoding mutated Homo sapiens v-Ki-ras2 Kirsten rat sarcoma viral oncogene homolog (KRAS), transcript variant a, mutation KRAS_G13V. The coding region ranges from nucleotide 182 to nucleotide 751.

### SEQ ID No. 24:

Amino acid sequence of mutated Homo sapiens v-Ki-ras2 Kirsten rat sarcoma viral oncogene homolog (KRAS), mutation KRAS_G13V

### SEQ ID No. 25:

Nucleotide sequence encoding mutated Homo sapiens v-Ki-ras2 Kirsten rat sarcoma viral oncogene homolog (KRAS), transcript variant a, mutation KRAS_A59T. The coding region ranges from nucleotide 182 to nucleotide 751.

### SEQ ID No. 26:

Amino acid sequence of mutated Homo sapiens v-Ki-ras2 Kirsten rat sarcoma viral oncogene homolog (KRAS), mutation. KRAS_A59T

SEQ ID No. 27: Nucleotide sequence encoding mutated Homo sapiens v-Ki-ras2 Kirsten rat sarcoma viral oncogene homolog (KRAS), transcript variant a, mutation KRAS_Q61E. The coding region ranges from nucleotide 182 to nucleotide 751.

### SEQ ID No. 28:

Amino acid sequence of mutated Homo sapiens v-Ki-ras2 Kirsten rat sarcoma viral oncogene homolog (KRAS), mutation KRAS_Q61E. The coding region ranges from nucleotide 182 to nucleotide 751.

### SEQ ID No. 29:

Nucleotide sequence encoding mutated Homo sapiens v-Ki-ras2 Kirsten rat sarcoma viral oncogene homolog (KRAS), transcript variant a, mutation KRAS_Q61H. The coding region ranges from nucleotide 182 to nucleotide 751.

### SEQ ID No. 30:

Amino acid sequence of mutated Homo sapiens v-Ki-ras2 Kirsten rat sarcoma viral oncogene homolog (KRAS), mutation KRAS_Q61H

### SEQ ID No. 31:

Nucleotide sequence encoding mutated Homo sapiens v-Ki-ras2 Kirsten rat sarcoma viral oncogene homolog (KRAS), transcript variant a, mutation KRAS_Q61H. The coding region ranges from nucleotide 182 to nucleotide 751.

### SEQ ID No. 32:

Amino acid sequence of mutated Homo sapiens v-Ki-ras2 Kirsten rat sarcoma viral oncogene homolog (KRAS), mutation KRAS_Q61H

### SEQ ID No. 33:

Nucleotide sequence encoding mutated Homo sapiens v-Ki-ras2 Kirsten rat sarcoma viral oncogene homolog (KRAS), transcript variant a, mutation KRAS_Q61K. The coding region ranges from nucleotide 182 to nucleotide 751.

### SEQ ID No. 34:

Amino acid sequence of mutated Homo sapiens v-Ki-ras2 Kirsten rat sarcoma viral oncogene homolog (KRAS), mutation KRAS_Q61K.

### SEQ ID No. 35:

Nucleotide sequence encoding mutated Homo sapiens v-Ki-ras2 Kirsten rat sarcoma viral oncogene homolog (KRAS), transcript variant a, mutation KRAS_Q61L. The coding region ranges from nucleotide 182 to nucleotide 751.

### SEQ ID No. 36:

Amino acid sequence of mutated Homo sapiens v-Ki-ras2 Kirsten rat sarcoma viral oncogene homolog (KRAS), mutation KRAS_Q61L

### SEQ ID No. 37:

Nucleotide sequence encoding mutated Homo sapiens v-Ki-ras2 Kirsten rat sarcoma viral oncogene homolog (KRAS), transcript variant a, mutation KRAS_Q61R. The coding region ranges from nucleotide 182 to nucleotide 751.

### SEQ ID No. 38:

Amino acid sequence of mutated Homo sapiens v-Ki-ras2 Kirsten rat sarcoma viral oncogene homolog (KRAS), mutation KRAS_Q61R

### SEQ ID No. 39:

Nucleotide sequence encoding mutated Homo sapiens v-Ki-ras2 Kirsten rat sarcoma viral oncogene homolog (KRAS), transcript variant a, mutation KRAS_Q61P. The coding region ranges from nucleotide 182 to nucleotide 751.

### SEQ ID No. 40:

Amino acid sequence of mutated Homo sapiens v-Ki-ras2 Kirsten rat sarcoma viral oncogene homolog (KRAS), mutation KRAS_Q61P

### SEQ ID No. 41:

Nucleotide sequence encoding mutated Homo sapiens v-Ki-ras2 Kirsten rat sarcoma viral oncogene homolog (KRAS), transcript variant b, mutation KRAS_G12V. The coding region ranges from nucleotide 182 to nucleotide 748.

### SEQ ID No. 42:

Amino acid sequence of mutated Homo sapiens v-Ki-ras2 Kirsten rat sarcoma viral oncogene homolog (KRAS), mutation KRAS_G12V

### SEQ ID No. 43:

Nucleotide sequence encoding mutated Homo sapiens v-Ki-ras2 Kirsten rat sarcoma viral oncogene homolog (KRAS), transcript variant b, mutation. KRAS_G12S. The coding region ranges from nucleotide 182 to nucleotide 748.

### SEQ ID No. 44:

Amino acid sequence of mutated Homo sapiens v-Ki-ras2 Kirsten rat sarcoma viral oncogene homolog (KRAS), mutation KRAS_G12S

### SEQ ID No. 45:

Nucleotide sequence encoding mutated Homo sapiens v-Ki-ras2 Kirsten rat sarcoma viral oncogene homolog (KRAS), transcript variant b, mutation KRAS_G12A. The coding region ranges from nucleotide 182 to nucleotide 748.

### SEQ ID No. 46:

Amino acid sequence of mutated Homo sapiens v-Ki-ras2 Kirsten rat sarcoma viral oncogene homolog (KRAS), mutation KRAS_G12A

### SEQ ID No. 47:

Nucleotide sequence encoding mutated Homo sapiens v-Ki-ras2 Kirsten rat sarcoma viral oncogene homolog (KRAS), transcript variant b, mutation KRAS_G12D. The coding region ranges from nucleotide 182 to nucleotide 748.

### SEQ ID No. 48:

Amino acid sequence of mutated Homo sapiens v-Ki-ras2 Kirsten rat sarcoma viral oncogene homolog (KRAS), mutation KRAS_G12D

### SEQ ID No. 49:

Nucleotide sequence encoding mutated Homo sapiens v-Ki-ras2 Kirsten rat sarcoma viral oncogene homolog (KRAS), transcript variant b, mutation KRAS_G12C. The coding region ranges from nucleotide 182 to nucleotide 748.

### SEQ ID No. 50:

Amino acid sequence of mutated Homo sapiens v-Ki-ras2 Kirsten rat sarcoma viral oncogene homolog (KRAS), mutation KRAS_G12C

### SEQ ID No. 51:

Nucleotide sequence encoding mutated Homo sapiens v-Ki-ras2 Kirsten rat sarcoma viral oncogene homolog (KRAS), transcript variant b, mutation KRAS_G12R. The coding region ranges from nucleotide 182 to nucleotide 748.

### SEQ ID No. 52:

Amino acid sequence of mutated Homo sapiens v-Ki-ras2 Kirsten rat sarcoma viral oncogene homolog (KRAS), mutation KRAS_G12R

### SEQ ID No. 53:

Nucleotide sequence encoding mutated Homo sapiens v-Ki-ras2 Kirsten rat sarcoma viral oncogene homolog (KRAS), transcript variant b, mutation KRAS_G12F. The coding region ranges from nucleotide 182 to nucleotide 748.

### SEQ ID No. 54:

Amino acid sequence of mutated Homo sapiens v-Ki-ras2 Kirsten rat sarcoma viral oncogene homolog (KRAS), mutation KRAS_G12F

### SEQ ID No. 55:

Nucleotide sequence encoding mutated Homo sapiens v-Ki-ras2 Kirsten rat sarcoma viral oncogene homolog (KRAS), transcript variant b, mutation KRAS_G13C. The coding region ranges from nucleotide 182 to nucleotide 748.

### SEQ ID No. 56:

Amino acid sequence of mutated Homo sapiens v-Ki-ras2 Kirsten rat sarcoma viral oncogene homolog (KRAS), mutation KRAS_G13C

### SEQ ID No. 57:

Nucleotide sequence encoding mutated Homo sapiens v-Ki-ras2 Kirsten rat sarcoma viral oncogene homolog (KRAS), transcript variant b, mutation KRAS_G13D. The coding region ranges from nucleotide 182 to nucleotide 748.

### SEQ ID No. 58:

Amino acid sequence of mutated Homo sapiens v-Ki-ras2 Kirsten rat sarcoma viral oncogene homolog (KRAS) mutation KRAS_G13D

### SEQ ID No. 59:

Nucleotide sequence encoding mutated Homo sapiens v-Ki-ras2 Kirsten rat sarcoma viral oncogene homolog (KRAS), transcript variant b, mutation KRAS_G13S. The coding region ranges from nucleotide 182 to nucleotide 748.

### SEQ ID No. 60:

Amino acid sequence of mutated Homo sapiens v-Ki-ras2 Kirsten rat sarcoma viral oncogene homolog (KRAS), mutation KRAS_G13S

### SEQ ID No. 61:

Nucleotide sequence encoding mutated Homo sapiens v-Ki-ras2 Kirsten rat sarcoma viral oncogene homolog (KRAS), transcript variant b, mutation KRAS_G13V. The coding region ranges from nucleotide 182 to nucleotide 748.

### SEQ ID No. 62:

Amino acid sequence of mutated Homo sapiens v-Ki-ras2 Kirsten rat sarcoma viral oncogene homolog (KRAS), mutation KRAS_G13V

### SEQ ID No. 63:

Nucleotide sequence encoding mutated Homo sapiens v-Ki-ras2 Kirsten rat sarcoma viral oncogene homolog (KRAS), transcript variant b, mutation KRAS_A59T. The coding region ranges from nucleotide 182 to nucleotide 748.

### SEQ ID No. 64:

Amino acid sequence of mutated Homo sapiens v-Ki-ras2 Kirsten rat sarcoma viral oncogene homolog (KRAS), mutation KRAS_A59T

### SEQ ID No. 65:

Nucleotide sequence encoding mutated Homo sapiens v-Ki-ras2 Kirsten rat sarcoma viral oncogene homolog (KRAS), transcript variant b, mutation KRAS_Q61E. The coding region ranges from nucleotide 182 to nucleotide 748.

### SEQ ID No. 66:

Amino acid sequence of mutated Homo sapiens v-Ki-ras2 Kirsten rat sarcoma viral oncogene homolog (KRAS), mutation KRAS_Q61E

### SEQ ID No. 67:

Nucleotide sequence encoding mutated Homo sapiens v-Ki-ras2 Kirsten rat sarcoma viral oncogene homolog (KRAS), transcript variant b, mutation KRAS_Q61H. The coding region ranges from nucleotide 182 to nucleotide 748.

### SEQ ID No. 68:

Amino acid sequence of mutated Homo sapiens v-Ki-ras2 Kirsten rat sarcoma viral oncogene homolog (KRAS), mutation KRAS_Q61H

### SEQ ID No. 69:

Nucleotide sequence encoding mutated. Homo sapiens v-Ki-ras2 Kirsten rat sarcoma viral oncogene homolog (KRAS), transcript variant b, mutation KRAS_Q61H. The coding region ranges from nucleotide 182 to nucleotide 748.

### SEQ ID No. 70:

Amino acid sequence of mutated Homo sapiens v-Ki-ras2 Kirsten rat sarcoma viral oncogene homolog (KRAS), mutation KRAS_Q61H

### SEQ ID No. 71:

Nucleotide sequence encoding mutated Homo sapiens v-Ki-ras2 Kirsten rat sarcoma viral oncogene homolog (KRAS), transcript variant b, mutation KRAS_Q61K. The coding region ranges from nucleotide 182 to nucleotide 748.

### SEQ ID No. 72:

Amino acid sequence of mutated Homo sapiens v-Ki-ras2 Kirsten rat sarcoma viral oncogene homolog (KRAS), mutation KRAS_Q61K

### SEQ ID No. 73:

Nucleotide sequence encoding mutated Homo sapiens v-Ki-ras2 Kirsten rat sarcoma viral oncogene homolog (KRAS), transcript variant b, mutation KRAS_Q61L. The coding region ranges from nucleotide 182 to nucleotide 748.

### SEQ ID No. 74:

Amino acid sequence of mutated Homo sapiens v-Ki-ras2 Kirsten rat sarcoma viral oncogene homolog (KRAS), mutation KRAS_Q61L

### SEQ ID No. 75:

Nucleotide sequence encoding mutated Homo sapiens v-Ki-ras2 Kirsten rat sarcoma viral oncogene homolog (KRAS), transcript variant b, mutation KRAS_Q61R. The coding region ranges from nucleotide 182 to nucleotide 748.

### SEQ ID No. 76:

Amino acid sequence of mutated Homo sapiens v-Ki-ras2 Kirsten rat sarcoma viral oncogene homolog (KRAS), mutation KRAS_Q61R

### SEQ ID No. 77:

Nucleotide sequence encoding mutated Homo sapiens v-Ki-ras2 Kirsten rat sarcoma viral oncogene homolog (KRAS), transcript variant b, mutation KRAS_Q61P. The coding region ranges from nucleotide 182 to nucleotide 748.

### SEQ ID No. 78:

Amino acid sequence of mutated Homo sapiens v-Ki-ras2 Kirsten rat sarcoma viral oncogene homolog (KRAS), mutation KRAS_Q61P

### SEQ ID No. 79:

Nucleotide sequence encoding mutated Homo sapiens BRAF, mutation BRAF_G464R. The coding region ranges from nucleotide 62 to nucleotide 2362.

### SEQ ID No. 80:

Amino acid sequence of mutated Homo sapiens BRAF, mutation BRAF_G464R

### SEQ ID No. 81:

Nucleotide sequence encoding mutated. Homo sapiens BRAF, mutation BRAF_G464V. The coding region ranges from nucleotide 62 to nucleotide 2362.

### SEQ ID No. 82:

Amino acid sequence of mutated Homo sapiens BRAF, mutation BRAF_G1464V

### SEQ ID No. 83:

Nucleotide sequence encoding mutated Homo sapiens BRAF, mutation BRAF_G466A. The coding region ranges from nucleotide 62 to nucleotide 2362.

### SEQ ID No. 84:

Amino acid sequence of mutated Homo sapiens BRAF, mutation BRAF_G466A.

### SEQ ID No. 85:

Nucleotide sequence encoding mutated Homo sapiens BRAF, mutation BRAF The coding region ranges from nucleotide 62 to nucleotide 2362.

### SEQ ID No. 86:

Amino acid sequence of mutated Homo sapiens BRAF, mutation BRAF_G466R

### SEQ ID No. 87:

Nucleotide sequence encoding mutated Homo sapiens BRAF, mutation BRAF_F468C. The coding region ranges from nucleotide 62 to nucleotide 2362.

### SEQ ID No. 88:

Amino acid sequence of mutated Homo sapiens BRAF, mutation BRAF_F468C

### SEQ ID No. 89:

Nucleotide sequence encoding mutated Homo sapiens BRAF, mutation BRAF_G469R. The coding region ranges from nucleotide 62 to nucleotide 2362.

### SEQ ID No. 90:

Amino acid sequence of mutated Homo sapiens BRAF, mutation BRAF_G469R

### SEQ ID No. 91:

Nucleotide sequence encoding mutated Homo sapiens BRAF, mutation BRAF_G469R. The coding region ranges from nucleotide 62 to nucleotide 2362.

### SEQ ID No. 92:

Amino acid sequence of mutated Homo sapiens BRAF, mutation BRAF_G469R.

### SEQ ID No. 93:

Nucleotide sequence encoding mutated Homo sapiens BRAF, mutation BRAF_G469S. The coding region ranges from nucleotide 62 to nucleotide 2362.

### SEQ ID No. 94:

Amino acid sequence of mutated Homo sapiens BRAF, mutation BRAF_G469S

### SEQ ID No. 95:

Nucleotide sequence encoding mutated Homo sapiens BRAF, mutation BRAF_G464E. The coding region ranges from nucleotide 62 to nucleotide 2362.

### SEQ ID No. 96:

Amino acid sequence of mutated Homo sapiens BRAF, mutation BRAF_G464E

### SEQ ID No. 97:

Nucleotide sequence encoding mutated Homo sapiens BRAF, mutation BRAF_G466V. The coding region ranges from nucleotide 62 to nucleotide 2362.

### SEQ ID No. 98:

Amino acid sequence of mutated Homo sapiens BRAF, mutation BRAF_G466V

### SEQ ID No. 99:

Nucleotide sequence encoding mutated Homo sapiens BRAF, mutation BRAF_G466E. The coding region ranges from nucleotide 62 to nucleotide 2362.

### SEQ ID No. 100:

Amino acid sequence of mutated Homo sapiens BRAF, mutation BRAF_G466E

### SEQ ID No. 101:

Nucleotide sequence encoding mutated Homo sapiens BRAF, mutation BRAF_G469A. The coding region ranges from nucleotide 62 to nucleotide 2362.

### SEQ ID No. 102:

Amino acid sequence of mutated Homo sapiens BRAF, mutation BRAF_G469A

### SEQ ID No. 103:

Nucleotide sequence encoding mutated Homo sapiens BRAF, mutation BRAF_G469E. The coding region ranges from nucleotide 62 to nucleotide 2362.

### SEQ ID No. 104:

Amino acid sequence of mutated Homo sapiens BRAF, mutation BRAF_G469E

### SEQ ID No. 105:

Nucleotide sequence encoding mutated Homo sapiens BRAF, mutation BRAF_G469V. The coding region ranges from nucleotide 62 to nucleotide 2362.

### SEQ ID No. 106:

Amino acid sequence of mutated Homo sapiens BRAF, mutation BRAF_{—}0469V

### SEQ ID No. 107:

Nucleotide sequence encoding mutated Homo sapiens BRAF, mutation BRAF_F595L. The coding region ranges from nucleotide 62 to nucleotide 2362.

### SEQ ID No. 108:

Amino acid sequence of mutated Homo sapiens BRAF, mutation BRAF_F595L

### SEQ ID No. 109:

Nucleotide sequence encoding mutated Homo sapiens BRAF, mutation BRAF_G596R. The coding region ranges from nucleotide 62 to nucleotide 2362.

### SEQ ID No. 110:

Amino acid sequence of mutated Homo sapiens BRAF, mutation BRAF_G596R

### SEQ ID No. 111:

Nucleotide sequence encoding mutated Homo sapiens BRAF, mutation BRAF_L597V. The coding region ranges from nucleotide 62 to nucleotide 2362.

### SEQ ID No. 112:

Amino acid sequence of mutated Homo sapiens BRAF, mutation BRAF_L597V

### SEQ ID No. 113:

Nucleotide sequence encoding mutated Homo sapiens BRAF, mutation BRAF_L597S. The coding region ranges from nucleotide 62 to nucleotide 2362.

### SEQ ID No. 114:

Amino acid sequence of mutated Homo sapiens BRAF, mutation BRAF_L597S

### SEQ ID No. 115:

Nucleotide sequence encoding mutated Homo sapiens BRAF, mutation BRAF_V600E. The coding region ranges from nucleotide 62 to nucleotide 2362.

### SEQ ID No. 116:

Amino acid sequence of mutated Homo sapiens BRAF, mutation BRAF_V600E

### SEQ ID No. 117:

Nucleotide sequence encoding mutated Homo sapiens BRAF, mutation BRAF_V600K. The coding region ranges from nucleotide 62 to nucleotide 2362.

### SEQ ID No. 118:

Amino acid sequence of mutated Homo sapiens BRAF, mutation BRAF_V600K

### SEQ ID No. 119:

Nucleotide sequence encoding mutated Homo sapiens BRAF, mutation BRAF_V600R. The coding region ranges from nucleotide 62 to nucleotide 2362.

### SEQ ID No. 120:

Amino acid sequence of mutated Homo sapiens BRAF, mutation BRAF_V600R

### SEQ ID No. 121:

Nucleotide sequence encoding mutated Homo sapiens BRAF, mutation BRAF_K601N. The coding region ranges from nucleotide 62 to nucleotide 2362.

### SEQ ID No. 122:

Amino acid sequence of mutated Homo sapiens BRAF, mutation BRAF_K601N

### SEQ ID No. 123:

Nucleotide sequence encoding mutated Homo sapiens BRAF, mutation BRAF_K601E. The coding region ranges from nucleotide 62 to nucleotide 2362.

### SEQ ID No. 124:

Amino acid sequence of mutated Homo sapiens BRAF, mutation BRAF_K601E

### SEQ ID No. 125:

Nucleotide sequence encoding mutated Homo sapiens BRAF, mutation BRAF_D594G. The coding region ranges from nucleotide 62 to nucleotide 2362.

### SEQ ID No. 126:

Amino acid sequence of mutated Homo sapiens BRAF, mutation BRAF_D594G

### SEQ ID No. 127:

Nucleotide sequence encoding mutated Homo sapiens BRAF, mutation BRAF_D594V. The coding region ranges from nucleotide 62 to nucleotide 2362.

### SEQ ID No. 128:

Amino acid sequence of mutated Homo sapiens BRAF, mutation BRAF_D594V

### SEQ ID No. 129:

Nucleotide sequence encoding mutated Homo sapiens BRAF, mutation BRAF_L597R. The coding region ranges from nucleotide 62 to nucleotide 2362.

### SEQ ID No. 130:

Amino acid sequence of mutated Homo sapiens BRAF, mutation BRAF_L597R

### SEQ ID No. 131:

Nucleotide sequence encoding mutated Homo sapiens BRAF, mutation BRAF_L597Q. The coding region ranges from nucleotide 62 to nucleotide 2362.

### SEQ ID No. 132:

Amino acid sequence of mutated Homo sapiens BRAF, mutation BRAF_L597Q.

### SEQ ID No. 133:

Nucleotide sequence encoding mutated Homo sapiens BRAF, mutation BRAF_T599I. The coding region ranges from nucleotide 62 to nucleotide 2362.

### SEQ ID No. 134:

Amino acid sequence of mutated Homo sapiens BRAF, mutation BRAF_T599I

### SEQ ID No. 135:

Nucleotide sequence encoding mutated Homo sapiens BRAF, mutation BRAF_V6001. The coding region ranges from nucleotide 62 to nucleotide 2362.

### SEQ ID No. 136:

Amino acid sequence of mutated Homo sapiens BRAF, mutation BRAF_V600L

### SEQ ID No. 137:

Nucleotide sequence encoding Homo sapiens heat shock protein 90 kDa alpha (cytosolic), class A member 1 (HSP90AA1/HSP90), transcript variant 1, mRNA (>gi|153792589|ref|NM_{—}001017963.2|)

### SEQ ID No. 138:

Amino acid sequence of Homo sapiens heat shock protein 90 kDa alpha (cytosolic), class A member 1 isoform 1 (HSP90AA1) (>gi|153792590|ret|NP_{—}001017963.2|)

### SEQ ID No. 139:

Nucleotide sequence encoding Homo sapiens heat shock protein 90 kDa alpha (cytosolic), class A member 1 (HSP90AA1), transcript variant 2, mRNA (>gi|154146190|ref|NM_{—}005348.3|)

### SEQ ID No. 140:

Amino acid sequence of Homo sapiens heat shock protein 90 kDa alpha (cytosolic), class A member isoform 2 (>gi|154146191|ref|NP_{—}005339.3|)

### SEQ ID No. 141:

Nucleotide sequence encoding Homo sapiens heat shock protein 90 kDa alpha (cytosolic), class B member 1 (HSP90AB1), mRNA (>gi|20149593|ref|NM_{—}007355.21) mRNA

### SEQ ID No. 142:

Amino acid sequence of Homo sapiens heat shock 90 kDa protein 1, beta (>gi|20149594ref|NP_{—}031381.2|)

### SEQ ID No. 143:

Nucleotide sequence encoding Homo sapiens heat shock protein 90 kDa beta (Grp94), member 1 (HSP90B1), mRNA (>gi|4507676|ref|NM_{—}003299.1|)

### SEQ ID No. 144:

Amino acid sequence of Homo sapiens heat shock protein 90 kDa beta, member 1 (>gi|4507677|ref|NP_{—}003290.1|).

### SEQ ID No. 145:

Nucleotide sequence encoding Homo sapiens heat shock protein 90 kDa alpha (cytosolic), class A member 2 (HSP90AA2), mRNA (>gi|92859629|ref|NM_{—}001040141.1|)

### SEQ ID No. 146:

Amino acid sequence of Homo sapiens heat shock 90 kDa protein 1, alpha-like 3 (>gi|92859630|ref|NP_{—}001035231.1|)

### SEQ ID No. 147:

Nucleotide sequence encoding Homo sapiens v-raf murine sarcoma viral oncogene homolog B1 (BRAF). mRNA >gi|187608632|ref|NM_{—}004333.4|). The coding region ranges from nucleotide 62 to nucleotide 2362.

### SEQ ID No. 148:

Amino acid sequence of Homo sapiens v-raf murine sarcoma viral oncogene homolog B1 (BRAF), mRNA >gi|87608632|ref|NM_{—}004333.4|).

### SEQ ID No. 149:

Nucleotide sequence encoding Homo sapiens epidermal growth factor receptor (erythroblastic leukemia viral (v-erb-b) oncogene homolog, avian) (EGFR), transcript variant 1 (isoform a), mRNA (>gi|41327737|ref|NM_{—}005228.3|). The coding region ranges from nucleotide 247 to nucleotide 3879.

### SEQ ID No. 150:

Amino acid sequence of Homo sapiens epidermal growth factor receptor (erythroblastic leukemia viral (v-erb-b) oncogene homolog, avian) (EGFR), (isoform a).

### SEQ ID No. 151:

Nucleotide sequence encoding Homo sapiens epidermal growth factor receptor (erythroblastic leukemia viral (v-erb-b) oncogene homolog, avian) (EGFR), transcript variant 2 (isoform h), mRNA (>|NM_{—}201282.1|). The coding region ranges from nucleotide 247 to nucleotide 2133.

### SEQ ID No. 152:

Amino acid sequence of Homo sapiens epidermal growth factor receptor (erythroblastic leukemia viral (v-erb-b) oncogene homolog, avian) (EGFR), (isoform b).

### SEQ ID No. 153:

Nucleotide sequence encoding Homo sapiens epidermal growth factor receptor (erythroblastic leukemia viral (v-erb-b) oncogene homolog, avian) (EGFR), transcript variant 3 (isoform c). mRNA (>|NM_{—}201283.3|). The coding region ranges from nucleotide 247 to nucleotide 1464.

### SEQ ID No. 154:

Amino acid sequence of Homo sapiens epidermal growth factor receptor (erythroblastic leukemia viral (v-erb-b) oncogene homolog, avian) (EGFR), (isoform c).

### SEQ ID No. 155:

Nucleotide sequence encoding Homo sapiens epidermal growth factor receptor (erythroblastic leukemia viral (v-erb-b) oncogene homolog, avian) (EGFR), transcript variant 1 (isoform d), mRNA (>|NM_{—}201284.3|). The coding region ranges from nucleotide 247 to nucleotide 2364.

### SEQ ID No. 156:

Amino acid sequence of Homo sapiens epidermal growth factor receptor (erythroblastic leukemia viral (v-erb-b) oncogene homolog, avian) (EGFR), (isoform d).

### SEQ ID No. 157:

Nucleotide sequence encoding Homo sapiens v-Ki-ras2 Kirsten rat sarcoma viral oncogene homolog (KRAS), transcript variant a (isoform a), mRNA (|NM_{—}33360.2|). The coding region ranges from nucleotide 182 to nucleotide 751.

### SEQ ID No. 158:

Amino acid sequence of Homo sapiens v-Ki-ras2 Kirsten rat sarcoma viral oncogene homolog (KRAS) (isoform a).

### REFERENCES CITED HEREIN

Arao T et al., Small in-frame deletion in the epidermal growth factor receptor as a target for ZD6474. Cancer Res 64: 9101-4 (2004).
Aviel-Ronen S et al., K-ras mutations in non-small-cell lung carcinoma: a review. Clin Lung Cancer 8: 30-8 (2006).
Bamford S et al., The COSMIC (Catalogue of Somatic Mutations in Cancer) database and website. Br J Cancer 91: 355-8 (2004).
Banerji U et al., BRAF and NRAS mutations in melanoma: potential relationships to clinical response to HSP90 inhibitors. Mol Cancer Ther 7: 737-9 (2008).
Basso A D et al., Akt forms an intracellular complex with heat shock protein 90 (Hsp90) and Cdc37 and is destabilized by inhibitors of Hsp90 function. J Biol Chem 277: 39858-66 (2002).
Beroukhim R et al., Assessing the significance of chromosomal aberrations in cancer: methodology and application to glioma. Proc Natl Acad Sci USA 104: 20007-12 (2007).
Bhattacharjee A et al., Classification of human lung carcinomas by mRNA expression profiling reveals distinct adenocarcinoma subclasses. Proc Natl Acad Sci USA 98: 13790-5 (2001).
Bild A H et al., Oncogenic pathway signatures in human cancers as a guide to targeted therapies. Nature 439: 353-7 (2006).
Cappuzzo F et al., Epidermal growth factor receptor gene and protein and gefitinib sensitivity in non-small-cell lung cancer. J Natl Cancer Inst 97: 643-55 (2005).
Chen C F et al., Molecular genetic evidence supporting a novel human hepatocellular carcinoma tumor suppressor locus at 13q12.11. Genes Chromosomes Cancer 44: 320-8 (2005).
Choi K et al., Transcriptional profiling of non-small cell lung cancer cells with activating EGFR somatic mutations. PLoS ONE 2: e1226 (2007).
Druker B J et al., Activity of a specific inhibitor of the BCR-ABL tyrosine kinase in the blast crisis of chronic myeloid leukemia and acute lymphoblastic leukemia with the Philadelphia chromosome. N Engl J Med 344: 1038-42 (2001a).
Druker B J et al., Efficacy and safety of a specific inhibitor of the BCR-ABL tyrosine kinase in chronic myeloid leukemia. N Engl J Med 344: 1031-7 (2001b).
Eberhard D A et al., Mutations in the Epidermal Growth Factor Receptor and in KRAS Are Predictive and Prognostic Indicators in Patients With Non-Small-Cell Lung Cancer Treated With Chemotherapy Alone and in Combination With Erlotinib. J Clin Oncol (2005a).
Eberhard D A et al., Mutations in the epidermal growth factor receptor and in KRAS are predictive and prognostic indicators in patients with non-small-cell lung cancer treated with chemotherapy alone and in combination with erlotinib. J Clin Oncol 23: 5900-9 (2005b).
Garraway L A et al., Integrative genomic analyses identify MITF as a lineage survival oncogene amplified in malignant melanoma. Nature 436: 117-22 (2005).
Golub T R et al., Molecular classification of cancer: class discovery and class prediction by gene expression monitoring. Science 286: 531-7 (1999).
Grbovic O M, V600E B-Raf requires the Hsp90 chaperone for stability and is degraded in response to Hsp90 inhibitors. Proc Natl Acad Sci USA 103: 57-62 (2006).
Greshock J et al., Cancer cell lines as genetic models of their parent histology: analyses based on array comparative genomic hybridization. Cancer Res 67: 3594-600 (2007).
Greulich H et al., Oncogenic Transformation by Inhibitor-Sensitive and -Resistant EGFR Mutants. PLoS Med 2: e313 (2005).
Heinrich M C et al., Kinase mutations and imatinib response in patients with metastatic gastrointestinal stromal tumor. J Clin Oncol 21: 4342-9 (2003).
Hirsch F R et al., Increased epidermal growth factor receptor gene copy number detected by fluorescence in situ hybridization associates with increased sensitivity to gefitinib in patients with bronchioloalveolar carcinoma subtypes: a Southwest Oncology Group Study. J Clin Oncol 23: 6838-45 (2005).
Hostein I et al., Inhibition of signal transduction by the Hsp90 inhibitor 17-allylamino-17-demethoxygeldanamycin results in cytostasis and apoptosis. Cancer Res 61: 4003-9 (2001).
Hummel M et al., A biologic definition of Burkitt's lymphoma from transcriptional and genomic profiling N Engl J Med 354: 2419-30 (2006).
Hupe P et al., Analysis of array CGH data: from signal ratio to gain and loss of DNA regions. Bioinformatics 20: 3413-22 (2004).
Jong K et al., Cross-platform array comparative genomic hybridization meta-analysis separates hematopoietic and mesenchymal from epithelial tumors. Oncogene 26: 1499-506 (2007).
Karaman M W et al., Herrgard S. Treiber D K, Gallant P. Atteridge C E, Campbell B T et al (2008). A quantitative analysis of kinase inhibitor selectivity. Nat Biotechnol 26: 127-32 (2008).
Kaye F J A curious link between epidermal growth factor receptor amplification and survival: effect of “allele dilution” on gefitinib sensitivity? J Natl Cancer Inst 97: 621-3 (2005).
Kendall J et al., Oncogenic cooperation and coamplification of developmental transcription factor genes in lung cancer. Proc Natl. Acad Sci USA 104: 16663-8 (2007).
Knowles P P et al., Structure and chemical inhibition of the RET tyrosine kinase domain. J Biol Chem 281: 33577-87 (2006).
Kobayashi S et al., EGFR mutation and resistance of non-small-cell lung cancer to gefitinib. N Engl J Med 352: 786-92 (2005).
Lamb J et al., A mechanism of cyclin D1 action encoded in the patterns of gene expression in human cancer. Cell 114: 323-34 (2003).
Lenburg M E et al., Previously unidentified changes in renal cell carcinoma gene expression identified by parametric analysis of microarray data. BMC Cancer 3: 31 (2003).
Lin W M et al., Modeling genomic diversity and tumor dependency in malignant melanoma Cancer Res 68: 664-73 (2008).
Lockwood W W et al., DNA amplification is a ubiquitous mechanism of oncogene activation in lung and other cancers. Oncogene. (2008).
Lu Y et al., A gene expression signature predicts survival of patients with stage I non-small cell lung cancer. PLoS Med 3: e467 (2006).
Lynch T J et al., Activating mutations in the epidermal growth factor receptor underlying responsiveness of non-small-cell lung cancer to gefitinib. N Engl J Med 350: 2129-39 (2004).
McDermott U et al., Identification of genotype-correlated sensitivity to selective kinase inhibitors by using high-throughput tumor cell line profiling. Proc Natl Acad Sci USA 104: 19936-41 (2007).
Neve R M et al., A collection of breast cancer cell lines for the study of functionally distinct cancer subtypes. Cancer Cell 10: 515-27 (2006).
Nevins J R and Potti A, Mining gene expression profiles: expression signatures as cancer phenotypes. Nat Rev Genet 8: 601-9 (2007).
O'Reilly K E et al., mTOR inhibition induces upstream receptor tyrosine kinase signaling and activates Akt. Cancer Res 66: 1500-8 (2006).
Paez J G et al., EGFR mutations in lung cancer: correlation with clinical response to gefitinib therapy. Science 304: 1497-500 (2004).
Pao W et al., EGF receptor gene mutations are common in lung cancers from “never smokers” and are associated with sensitivity of tumors to gefitinib and erlotinib. Proc Natl Acad Sci USA 101: 13306-11 (2004).
Pao W et al., Acquired resistance of lung adenocarcinomas to gefitinib or erlotinib is associated with a second mutation in the EGFR kinase domain. PLoS Med 2: e73 (2005a).
Pao W et al., KRAS mutations and primary resistance of lung adenocarcinomas to gefitinib or erlotinib. PLoS Med 2: e17 (2005b).
Papadopoulos N et al., The role of companion diagnostics in the development and use of mutation-targeted cancer therapies. Nat Biotechnol 24: 985-95 (2006).
Reich M et al., GenePattern 2.0. Nat Genet. 38: 500-1 (2006).
Rinaldi A et al., Genomic and expression profiling identifies the B-cell associated tyrosine kinase Syk as a possible therapeutic target in mantle cell lymphoma. Br J Haematol 132: 303-16 (2006).
Schmid M et al., Homozygous deletions of methylthioadenosine phosphorylase (MTAP) are more frequent than p16INK4A (CDKN2) homozygous deletions in primary non-small cell lung cancers (NSCLC). Oncogene 17: 2669-75 (1998).
Shah N P et al., Overriding imatinib resistance with a novel ABL kinase inhibitor. Science 305: 399-401 (2004).
Shankavaram U T et al., Transcript and protein expression profiles of the NCI-60 cancer cell panel: an integromic microarray study. Mol Cancer Ther 6: 820-32 (2007).
Sharma S V et al., Epidermal growth factor receptor mutations in lung cancer. Nat Rev Cancer 7: 169-81 (2007).
Sharp S and Workman P, Inhibitors of the HSP90 molecular chaperone: current status. Adv Cancer Res 95: 323-48 (2006).
Shendure J et al., Advanced sequencing technologies: methods and goals Nat Rev Genet 5: 335-44 (2004).
Shimamura T et al., Non-small-cell lung cancer and Ba/F3 transformed cells harboring the ERBB2 G776insV_G/C mutation are sensitive to the dual-specific epidermal growth factor receptor and ERBB2 inhibitor HKI-272. Cancer Res 66: 6487-91 (2006).
Shoemaker R H The NCI60 human tumour cell line anticancer drug screen. Nat Rev Cancer 6: 813-23 (2006).
Slamon D J et al., Use of chemotherapy plus a monoclonal antibody against HER2 for metastatic breast cancer that overexpresses HER2. N Engl J Med 344: 783-92 (2001).
Solit D B et al., BRAD mutation predicts sensitivity to MEK inhibition. Nature 439: 358-62 (2006).
Solit D B and Rosen NHsp90: a novel target for cancer therapy. Curr Top Med Chem 6: 1205-14 (2006).
Song L et al., Dasatinib (BMS-354825) selectively induces apoptosis in lung cancer cells dependent on epidermal growth factor receptor signaling for survival. Cancer Res 66: 5542-8 (2006).
Sos M L et al., Expression of signaling mediators downstream of EGF-receptor predict sensitivity to small molecule inhibitors directed against the EGF-receptor pathway. J Thorac Oncol 3: 170-3 (2008).
Stamos J et al., Structure of the epidermal growth factor receptor kinase domain alone and in complex with a 4-anilinoquinazoline inhibitor. J Biol Chem 277: 46265-72 (2002).
Thomas R K, et al., High-throughput oncogene mutation profiling in human cancer. Nat. Genet. (2007).
Tracy S et al., Gefitinib induces apoptosis in the EGFRL858R non-small-cell lung cancer cell line H3255. Cancer Res 64: 7241-4 (2004).
Tsao M S et al., Erlotinib in lung cancer—molecular and clinical predictors of outcome. N Engl Med 353: 133-44 (2005).
Uphoff C C and Drexler H G. Eradication of mycoplasma contaminations Methods Mol Biol 290: 25-34 (2005).
Weir B A et al., Characterizing the cancer genome in lung adenocarcinoma. Nature 450: 893-8 (2007).
Yun C H et al., Structures of lung cancer-derived EGFR mutants and inhibitor complexes: mechanism of activation and insights into differential inhibitor sensitivity. Cancer Cell 11: 217-27 (2007).
Huang F et al., Identification of candidate molecular markers predicting sensitivity in solid tumors to dasatinib: rationale for patients selection. E. Cancer Res. March 1; 67(5):2226-38 (2007)
Jackson, E. L. et al., Analysis of lung tumor initiation and progression using conditional expression of oncogenic K-ras. Genes Dev 15:3243-3248 (2001)
Johnson L et al., Somatic activation of the K-ras oncogene causes early onset lung cancer in mice. Nature. 410(6832):1111-6 (2001).
Ji H. et al., LKB1 modulates lung cancer differentiation and metastasis. Nature. 448(7155):807-10. (2007)
Thomas R K et al., Sensitive mutation detection in heterogeneous cancer specimens by massively parallel picoliter reactor sequencing. Nat. Med. July; 12(7):852-5. Epub 2006 Jun. 25. Erratum in: Nat Med. 2006 October; 12(10):1220.

1. A method of selecting (a) cell(s), (a) tissue(s) or (a) cell culture(s) with susceptibility to an HSP90 inhibitor, comprising the steps:
(a) determining the presence of at least one activating mutation in the KRAS gene in said cell, tissue or cell culture; and
(b) selecting (a) cell(s), tissue(s) or cell culture(s) with at least one activating mutation in the KRAS gene.

2. The method of claim 1, further comprising the steps
(i) contacting said cell(s), tissue(s) or cell culture(s) with an HSP90 inhibitor; and
(ii) evaluating viability of said cell(s), tissue(s) or cell culture(s) contacted with an HSP90 inhibitor.

3. A method for determining the responsiveness of a mammalian tumor cell or cancer cell to treatment with an HSP90 inhibitor, said method comprising determining the presence of at least one activating mutation in the KRAS gene in said tumor cell, wherein said activating mutation is indicative of whether the cell is likely to respond or is responsive to the treatment.

4. The method of claim 3, whereby additionally an activating mutation in the EGFR and/or the BRAF gene is determined.

5. In vitro method for the identification of a responder for or a patient sensitive to an HSP90 inhibitor, said method comprising the following steps:
(a) obtaining a sample of a patient suspected to suffer from or being prone to suffer from a cancer characterized by the presence of at least one activating mutation in the KRAS gene, and, optionally, in the EGFR and/or the BRAF gene: and
(b) evaluating the presence of at least one activating mutation in the KRAS gene, and, optionally, the EGFR and/or the BRAF gene:
whereby an activating mutation in the KRAS gene alone or in addition to an activating mutation in the EGFR and/or the BRAF gene is indicative for a responding patient or is indicative for a sensitivity of said patient to an HSP90 inhibitor.

6.-8. (canceled)

9. The method of claim 5, wherein said mutation in the KRAS gene is selected from the group consisting of KRAS_G12C, KRAS_G12R, KRAS_G12D, KRAS_G12A, KRAS_G12S, KRAS_G12V, KRAS_G13D, KRAS_G13S, KRAS_G13C, KRAS_G13V, KRAS_Q61H, KRAS_Q61R, KRAS_Q61P, KRAS_Q61L, KRAS_Q61K, KRAS_Q61E, KRAS_A59T and KRAS_G12F.

10. The method of claim 5, wherein said mutation in the EGFR gene is selected from the group consisting of EGFR_D770_N771>AGG; EGFR_D770_N771insG; EGFR_D770_N771insG; EGFR_D770_N771 insN; EGFR_E709A; EGFR_E709G; EGFR_E709H, EGFR_E709K; EGFR_E709V; EGFR_E746_A750del; EGFR_E746_A750del, T751A; EGFR_E746_A750del, V ins; EGFR_E746_T751del, I ins; EGFR_E746_T751del; S752A; EGFR_E746_T751del, S752D; EGFR_E746_T751del, V ins; EGFR_G719A; EGFR_G719C; EGFR_G719S; EGFR_H773_V774insH; EGFR_H773_V774insNPH; EGFR_H773_V774insPH; EGFR_H773>NPY; EGFR_L747_E749del; EGFR_L747_E749del, A750P; EGFR_L747_S752del; EGFR_L747_S752del, P753S; EGFR_L747_S752del, Q ins; EGFR_L747_T750del, P ins; EGFR_L747_T751del; EGFR_L858R; EGFR_L861Q; EGFR_M766_A767insAI; EGFR_P772_H773insV; EGFR_S752_I759del; EGFR_S768I; EGFR_T790M; EGFR_V769_D770insASV; EGFR_V769_D770insASV; and EGFR_V774_C775insHV.

11. The method of claim 5, wherein said mutation in the BRAF gene is selected from the group consisting of BRAF_D594G, BRAF_D594V, BRAF_F468C, BRAF_F595L, BRAF_G464E, BRAF_G464R, BRAF_G464V, BRAF_G466A, BRAF_G466E, BRAF_G466R, BRAF_G466V, BRAF_G469A, BRAF_G469E, BRAF_G469R, BRAF_G469R, BRAF_G469S, BRAF_G469V, BRAF_G596R, BRAF_K601E, BRAF_K601N, BRAF_L597Q, BRAF_L597R, BRAF_L597S, BRAF_L597V, BRAF_T599I, BRAF_V600E, BRAF_V600K, BRAF_V600L, and BRAF_V600R.

12. The method of claim 5, wherein said mutation in the KRAS gene, the EGFR and/or the BRAF gene is detected by SSP, PCR-RFLP assay, real-time PCR, sequencing, HPLC or mass-spectrometric genotyping.

13. The method of claim 5, wherein said sample is obtained from a patient suspected to suffer from or being prone to suffer from cancer.

14. The method of claim 5, wherein the presence of the mutation is evaluated using an oligo- or polynucleotide capable of detecting (an) activating mutation(s) of at least one activating mutation in the KRAS gene and, optionally in the EGFR and/or in the BRAF gene.

15. The method of claim 14, wherein said oligonucleotide is about 15 to 100 nucleotides in length.

16. A method of monitoring the efficacy of a treatment of a cancer characterized by the presence of at least one activating mutation in the KRAS gene, and, optionally, in the EGFR and/or the BRAF gene in a subject/patient suffering from said disorder or being prone to suffering from said disorder comprising the steps:
a) determining in a cell or tissue sample from said subject/patient the expression or activity of KRAS, and, optionally the activity or expression level of EGFR and/or the activity or expression level of BRAF; and
b) comparing the activity of said at least one marker gene determined in a) with a reference or control expression level or reference or control activity of KRAS, and, optionally with a reference or control expression level of EGFR and/or with a reference or control expression level of BRAF,
wherein the extent of the difference between said activity or expression level determined in a) and said reference expression level or reference activity is indicative for said efficacy of a treatment of said cancer.

17. A method of predicting the efficacy of a treatment of a cancer characterized by the presence of at least one activating mutation in the KRAS gene, and, optionally, in the EGFR and/or the BRAF gene for a subject/patient suffering from said disorder or being prone to suffering from said disorder comprising the steps of
a) determining in a cell or tissue sample from said subject/patient the activity or expression level of at least one marker gene selected from the group consisting of KRAS, EGFR and/or BRAF; and
b) comparing the activity of said at least one marker gene determined in a) with a reference activity or reference expression level of said at least one marker gene, optionally determined in a cell or tissue sample obtained from a control subject/patient (responder and/or non-responder),
wherein the extent of the difference between said activity or expression level determined in a) and said reference or control activity or said reference or control expression level is indicative for the predicted efficacy of a treatment of cancer.

18. The method of claim 16, wherein said treatment of cancer characterized by the presence of at least one activating mutation in the KRAS gene, and, optionally, in the EGFR and/or the BRAF gene comprises the administration of an HSP90 inhibitor.

19. (canceled)

20. The method of claim 5, wherein said cancer is selected from the group consisting of non-small cell lung cancer, lung adenocarcinoma, pancreatic cancer, colorectal cancer, breast cancer, head and neck cancer, ovarian cancer, endometrial cancer, gastrointestinal cancer (including gastric and esophageal cancer), renal cell cancer, urinary tract carcinomas, leukemias, prostate cancer, lymphomas, melanomas, brain tumors, pediatric tumors and sarcomas.

21. A kit useful for carrying out the method of claim 3, comprising oligonucleotides or polynucleotides capable of determining the presence of at least one activating mutation in the KRAS gene, and, optionally, the EGFR gene and/or the BRAF gene.

22.-26. (canceled)

27. A method for the treatment of a patient having a KRAS positive cancer, the method comprising selecting a patient whose cancer is characterized by the presence of at least one activating mutation in the KRAS gene and treating said patient with an effective amount of an HSP90 inhibitor.

28. The method of claim 27, wherein said HSP90 inhibitor is geldanamycin or a derivative thereof.

29. The method of claim 28, wherein said geldanamycin derivative is 17-AAG or IPI-504.

30. The method of claim 27, wherein said HSP90 inhibitor is NVP-AUY922.

31. The method of claim 27, wherein said cancer is selected from the group consisting of non-small cell lung cancer, lung adenocarcinoma, pancreatic cancer, colorectal cancer, breast cancer, head and neck cancer, ovarian cancer, endometrial cancer, gastrointestinal cancer (including gastric and esophageal cancer), renal cell cancer, urinary tract carcinomas, leukemias, prostate cancer, lymphomas, melanomas, brain tumors, pediatric tumors and sarcomas.
